(19) United States
(12) Patent Application Publication
(10) Pub. No.: US 2026/0218267 A1
(43) Pub. Date: Jul. 30, 2026

(54) METHODS AND COMPOSITIONS FOR PRODUCTION AND PURIFICATION OF PEPTIDES

(71) Applicant: MICROPEP TECHNOLOGIES S.A., Auzeville-Tolosane (FR)

(72) Inventors: Orso SUBRINI, Auzeville-Tolosane (FR); Renaud CARTIER, Auzeville-Tolosane (FR); Mikaêl Stéphane COURBOT, Auzeville-Tolosane (FR)

(73) Assignee: MICROPEP TECHNOLOGIES S.A., Auzeville-Tolosane (FR)

(21) Appl. No.: 19/159,034

(22) PCT Filed: Feb. 23, 2024
(86) PCT No.: PCT/EP2024/054685

§ 371 Date: Aug. 22, 2025

Related U.S. Application Data
(60) Provisional application No. 63/486,652, filed on Feb. 23, 2023.
(60) Provisional application No. 63/486,653, filed on Feb. 23, 2023.

(51) Int. Cl. C12P 21/02; A01N 37/46; A01P 15/00; C07K 14/435; C12N 1/20

(52) U.S. Cl. CPC C12P 21/02; A01N 37/46; A01P 15/00; C07K 14/435; C12N 1/20; C07K 2319/01

(57) ABSTRACT

Provided herein are methods of producing peptides from inclusion bodies by linking two or more oligopeptides to an insoluble carrier polypeptide like onconase or TAF12, wherein the oligopeptides are released from the insoluble carrier protein by sequence-specific, chemical cleavage of the peptide bond using acids like acetic acid. Also provided herein are onconase variants with improved properties.

### CROSS-REFERENCE TO RELATED APPLICATIONS

This application claims the benefit of U.S. Provisional Patent Application Nos. 63/486,652, 63/486,653, and 63/486,654, all of which were filed on Feb. 23, 2023, and the entire contents of each of which are incorporated herein by reference.

### REFERENCE TO AN ELECTRONIC SEQUENCE LISTING

The contents of the electronic sequence listing (185952001040SEQLIST.xml; Size: 3,250,395 bytes; and Date of Creation: Feb. 7, 2024) is herein incorporated by reference in its entirety.

### FIELD OF INVENTION

The present invention relates to methods of expressing and purifying peptides from inclusion bodies.

### BACKGROUND

Although short peptides can be produced in high yields via chemical synthesis (Merrifield, R. B., (1993) J. Am. Chem. Soc. 85:2149-2154), recombinant production offers the potential for large scale production at a more reasonable cost. However, the bioproduction of short peptides present particular challenges. Peptides shorter than 30 amino acids in length are usually unstructured, and are thus difficult to express alone by recombinant DNA technologies in microbial cultures. Short peptide production by recombinant means is further complicated by potential toxicity to the host cell or by their high susceptibility to in vivo proteolysis, which leads to low, if any, yield of the peptides (Gottesman, S. (1990) Methods in Enzymology 185:119-129; Goff and Goldberg, (1986) in Maximizing Gene Expression p 287-314). Though the fusion of short peptides to larger carrier proteins can help to reduce toxicity, provide stability, and aid in purification (Terpe, K. (2003) Appl. Microbiol. Biotechnol. 60:523-533), even when the peptide is part of a larger fusion protein, high concentrations of fusion peptide in a cell can lead to toxic effects.

The use of an insoluble carrier protein as a fusion partner can help mitigate the toxicity and proteolytic degradation of the peptides by promoting their aggregation into insoluble inclusion bodies. Inclusion bodies protect against proteolysis and serve as a purification substrate (T. Kempe et al., (1985) Gene 39:239-245). However, common methods for separating peptides from the carrier protein can be inefficient and generally requires costly cleavage reagents and affinity columns for purification. For example, protease-based recovery using site-specific proteases or autoprotease fusion partners to separate the peptides from the carrier protein has been used with moderate success, but these strategies require large peptide fusions and are not compatible with concatemeric peptide production strategies.

Peptide production methods using onconase as an insoluble carrier protein have been developed and present a potential strategy to produce peptides in bacteria or yeast. One of these onconase-based strategies was recently described by Pane, K., et al. (2016) PLoS One. 11(1):e0146552 (“Pane, et al.”). Pane et al. used an onconase-peptide fusion construct to express and purify peptides from inclusion bodies in E. coli. Separation of the peptides from the onconase was performed using a chemical cleavage strategy, which can be performed using low-cost reagents and generally targets peptide bonds present at low frequency in proteins. Notably, though the protocol described by Pane et al. required the use of chaotropic agents but precluded the use of a chromatographic step. The protocol still resulted in low peptide yield and purity, which can potentially compromise downstream applications of peptides produced by this method.

Thus, there remains a need for methods of production of short peptides that avoid toxicity and proteolytic degradation, as well as allow for simple, inexpensive and efficient recovery of the peptides.

### SUMMARY OF INVENTION

Aspect 1: A method of producing oligopeptides comprising:
expressing a fusion polypeptide comprising an insoluble carrier polypeptide operably linked to two or more oligopeptides by a peptide bond, and
releasing the two or more oligopeptides from the insoluble carrier polypeptide by sequence-specific chemical cleavage of the peptide bond.

Aspect 2: The method of Aspect 1, wherein the insoluble carrier polypeptide comprises an onconase polypeptide.

Aspect 3: The method of Aspect 1, wherein the insoluble carrier polypeptide comprises a TAF12 polypeptide.

Aspect 4: The method of Aspect 1, wherein the insoluble carrier polypeptide comprises a trpΔLE polypeptide, a ketosteroid isomerase (KSI) polypeptide, a β-galactosidase polypeptide, a PagP polypeptide, a truncated E. coli PurF F4 fragment polypeptide, a Pseudomonas aeruginosa PaP3.30 polypeptide, a histone fold domain of the human transcription factor TAF12 (TAF12-HFD) polypeptide, a cleavable self-aggregating tag INTEIN-ELK16, a E. coli maltose-binding protein, an E. coli RNAse II polypeptide, an E. coli alkaline phosphatase polypeptide, an E. coli phospholipase A polypeptide, an E. coli D-lactamase polypeptide, a Salmonella typhimurium MalK protein, a Clostridium thermocellum endoglucanase D polypeptide, a Bacillus thuringiensis subsp. aizawai IPL7 insecticidal proteins, a human procathepsin B polypeptide, a porcine interferon-γ polypeptide, a T5 DNA polymerase polypeptide, and an E. coli thioredoxin polypeptide.

Aspect 5. The method of any one of Aspects 1-4, wherein the fusion polypeptide comprises three or more oligopeptides, four or more oligopeptides, five or more oligopeptides, six or more oligopeptides, eight or more oligopeptides, ten or more oligopeptides, fifteen or more oligopeptides, twenty or more oligopeptides.

Aspect 6: The method of any one of Aspects 1-5, wherein the two or more oligopeptides are different.

Aspect 7: The method of any one of Aspects 1-6, wherein all the oligopeptides are operably linked to the N-terminus of the insoluble carrier polypeptide or all the oligopeptides are operably linked to the C-terminus of the insoluble carrier polypeptide.

Aspect 8: The method of any one of Aspects 1-6, wherein at least one oligopeptide is operably linked to the N-terminus of the insoluble carrier polypeptide and at least one oligopeptide is operably linked to the C-terminus of the insoluble carrier polypeptide.

Aspect 9. The method of Aspect 8, wherein the fusion polypeptide comprises two or more oligopeptides, three or more oligopeptides, four or more oligopeptides, five or more oligopeptides, six or more oligopeptides, eight or more oligopeptides, ten or more oligopeptides operably linked to the N-terminus of the insoluble carrier polypeptide and/or two or more oligopeptides, three or more oligopeptides, four or more oligopeptides, five or more oligopeptides, six or more oligopeptides, eight or more oligopeptides, ten or more oligopeptides operably linked to the C-terminus of the insoluble carrier polypeptide.

Aspect 10: The method of any one of Aspects 1-9, wherein the peptide bond comprises (i) a methionine and the sequence-specific chemical cleavage is with cyanogen bromide, (ii) a tryptophan and the sequence-specific chemical cleavage is with BNPS-skatole, (iii) an aspartic acid-proline (Asp-Pro) bond and the sequence-specific chemical cleavage is with formic acid, (iv) an asparagine-glycine bond and the sequence-specific chemical cleavage is with hydroxylamine, or (v) a cysteine and the sequence-specific chemical cleavage is with NTCB.

Aspect 11: The method of any one of Aspects 1-9, wherein the peptide bond comprises an Asp-Pro bond and the sequence-specific chemical cleavage is with acetic acid.

Aspect 12: The method of any one of Aspects 1-11, wherein the oligopeptides are operably linked by the peptide bond and are released from each other when the oligopeptides are released from the insoluble carrier polypeptide.

Aspect 13: The method of any one of Aspects 1-11, wherein the oligopeptides are operably linked by a different peptide bond and are released from each other by sequence-specific chemical cleavage of the different peptide bond after the oligopeptides are released from the insoluble carrier polypeptide.

Aspect 14: The method of Aspect 13, wherein the different peptide bond comprises (i) a methionine and the sequence-specific chemical cleavage is with cyanogen bromide, (ii) a tryptophan and the sequence-specific chemical cleavage is with BNPS-skatole, (iii) an aspartic acid-proline (Asp-Pro) bond and the sequence-specific chemical cleavage is with formic acid, (iv) an asparagine-glycine bond and the sequence-specific chemical cleavage is with hydroxylamine, or (v) a cysteine and the sequence-specific chemical cleavage is with NTCB.

Aspect 15: The method of Aspect 13, wherein the different peptide bond comprises an Asp-Pro bond and the sequence-specific chemical cleavage is with acetic acid.

Aspect 16: The method of any one of Aspects 1-15, wherein the oligopeptides are at least four amino acids long, at least five amino acids long, at least six amino acids long, at least seven amino acids long, at least eight amino acids long, at least nine amino acids long, at least ten amino acids long, at least fifteen amino acids long, at least twenty amino acids long, or at least twenty-five amino acids long.

Aspect 17: The method of any one of Aspects 1-16, wherein the oligopeptides are less than fifty amino acids long, less than forty-five amino acids long, less than forty amino acids long, less than thirty-five amino acids long, less than thirty amino acids long, less than twenty-five amino acids long, or less than twenty amino acids long.

Aspect 18: The method of any one of Aspects 1-15, wherein the oligopeptides are between four amino acids long and fifty amino acids long, between six amino acids long and forty amino acids long, between six amino acids long and thirty amino acids long, or between eight amino acids long and twenty-five amino acids long.

Aspect 19: The method of any one of Aspect 1-18, wherein the fusion peptide is expressed in bacteria or yeast.

Aspect 20: The method of Aspect 19, wherein the bacteria is E. coli or Vibrio natriegens

Aspect 21: The method of Aspect 19 or Aspect 20, wherein the yield of released oligopeptide is at least 10 mg/L of bacterial culture, at least 20 mg/L of bacterial culture, at least 30 mg/L of bacterial culture, at least 40 mg/L of bacterial culture, at least 50 mg/L of bacterial culture, at least 1 g/L or at least 5 g/L.

Aspect 22: A fusion polypeptide comprising an insoluble carrier polypeptide operably linked to two or more oligopeptides, wherein the operably linkage between the two or more oligopeptides and the insoluble carrier polypeptide comprise a peptide bond that is capable of sequence-specific chemical cleavage.

Aspect 23: The fusion polypeptide of Aspect 22, wherein the insoluble carrier polypeptide comprises an onconase polypeptide.

Aspect 24: The fusion polypeptide of Aspect 22, wherein the insoluble carrier polypeptide comprises a TAF12 polypeptide.

Aspect 25: The fusion polypeptide of Aspect 22, wherein the insoluble carrier polypeptide comprises a trpΔLE polypeptide, a ketosteroid isomerase (KSI) polypeptide, a β-galactosidase polypeptide, a PagP polypeptide, a truncated E. coli PurF F4 fragment polypeptide, a Pseudomonas aeruginosa PaP3.30 polypeptide, a histone fold domain of the human transcription factor TAF12 (TAF12-HFD) polypeptide, a cleavable self-aggregating tag INTEIN-ELK16, a E. coli maltose-binding protein, an E. coli RNAse II polypeptide, an E. coli alkaline phosphatase polypeptide, an E. coli phospholipase A polypeptide, an E. coli β-lactamase polypeptide, a Salmonella typhimurium MalK protein, a Clostridium thermocellum endoglucanase D polypeptide, a Bacillus thuringiensis subsp. aizawai IPL7 insecticidal proteins, a human procathepsin B polypeptide, a porcine interferon-γ polypeptide, a T5 DNA polymerase polypeptide, and an E. coli thioredoxin polypeptide.

Aspect 26. The fusion polypeptide of any one of Aspects 22-25, wherein the fusion polypeptide comprises three or more oligopeptides, four or more oligopeptides, five or more oligopeptides, six or more oligopeptides, eight or more oligopeptides, ten or more oligopeptides, fifteen or more oligopeptides, twenty or more oligopeptides.

Aspect 27: The fusion polypeptide of any one of Aspects 22-26, wherein the two or more oligopeptides are different.

Aspect 28: The fusion polypeptide of any one of Aspects 22-27, wherein all the oligopeptides are operably linked to the N-terminus of the insoluble carrier polypeptide or all the oligopeptides are operably linked to the C-terminus of the insoluble carrier polypeptide.

Aspect 29: The fusion polypeptide of any one of Aspects 22-27, wherein at least one oligopeptide is operably linked to the N-terminus of the insoluble carrier polypeptide and at least one oligopeptide is operably linked to the C-terminus of the insoluble carrier polypeptide.

Aspect 30. The fusion polypeptide of Aspect 29, wherein the fusion polypeptide comprises two or more oligopeptides, three or more oligopeptides, four or more oligopeptides, five or more oligopeptides, six or more oligopeptides, eight or more oligopeptides, ten or more oligopeptides operably linked to the N-terminus of the insoluble carrier polypeptide and/or two or more oligopeptides, three or more oligopeptides, four or more oligopeptides, five or more oligopeptides, six or more oligopeptides, eight or more oligopeptides, ten or more oligopeptides operably linked to the C-terminus of the insoluble carrier polypeptide.

Aspect 31: The fusion polypeptide of any one of Aspects 22-30, wherein the peptide bond comprises (i) a methionine and the sequence-specific chemical cleavage is with cyanogen bromide, (ii) a tryptophan and the sequence-specific chemical cleavage is with BNPS-skatole, (iii) an aspartic acid-proline (Asp-Pro) bond and the sequence-specific chemical cleavage is with formic acid, (iv) an asparagine-glycine bond and the sequence-specific chemical cleavage is with hydroxylamine, or (v) a cysteine and the sequence-specific chemical cleavage is with NTCB.

Aspect 32: The fusion polypeptide of any one of Aspects 22-30, wherein the peptide bond comprises an Asp-Pro bond and the sequence-specific chemical cleavage is with acetic acid.

Aspect 33: The fusion polypeptide of any one of Aspects 22-32, wherein the oligopeptides are operably linked by the peptide bond and are capable of release from each other with the sequence-specific chemical cleavage.

Aspect 34: The fusion polypeptide of any one of Aspects 22-32, wherein the oligopeptides are operably linked by a different peptide bond and are capable of release from each other by a different sequence-specific chemical cleavage.

Aspect 35: The fusion polypeptide of Aspect 34, wherein the different peptide bond comprises (i) a methionine and the different sequence-specific chemical cleavage is with cyanogen bromide, (ii) a tryptophan and the different sequence-specific chemical cleavage is with BNPS-skatole, (iii) an aspartic acid-proline (Asp-Pro) bond and the different sequence-specific chemical cleavage is with formic acid, (iv) an asparagine-glycine bond and the different sequence-specific chemical cleavage is with hydroxylamine, or (v) a cysteine and the different sequence-specific chemical cleavage is with NTCB.

Aspect 36: The fusion polypeptide of Aspect 34, wherein the different peptide bond comprises an Asp-Pro bond and the different sequence-specific chemical cleavage is with acetic acid.

Aspect 37: The fusion polypeptide of any one of Aspects 22-36, wherein the oligopeptides are at least four amino acids long, at least five amino acids long, at least six amino acids long, at least seven amino acids long, at least eight amino acids long, at least nine amino acids long, at least ten amino acids long, at least fifteen amino acids long, at least twenty amino acids long, or at least twenty-five amino acids long.

Aspect 38: The fusion polypeptide of any one of Aspects 22-37, wherein the oligopeptides are less than fifty amino acids long, less than forty-five amino acids long, less than forty amino acids long, less than thirty-five amino acids long, less than thirty amino acids long, less than twenty-five amino acids long, or less than twenty amino acids long.

Aspect 39: The fusion polypeptide of any one of Aspects 22-36, wherein the oligopeptides are between four amino acids long and fifty amino acids long, between six amino acids long and forty amino acids long, between six amino acids long and thirty amino acids long, or between eight amino acids long and twenty-five amino acids long.

Aspect 40: A method of releasing an oligopeptide fused to an insoluble carrier polypeptide, which forms inclusion bodies in a cell comprising
a) expressing a fusion polypeptide comprising the oligopeptide operably linked to the insoluble carrier polypeptide, wherein the operable linkage is a peptide bond that is capable of sequence-specific chemical cleavage by acetic acid,
b) purifying the inclusion bodies, and
c) incubating the inclusion bodies with acid at a temperature of greater than 50° C. for at least one hour, wherein the oligopeptide is released from the fusion polypeptide by sequence-specific cleavage of the peptide bond.

Aspect 41: The method of Aspect 40, wherein the insoluble carrier polypeptide comprises an onconase polypeptide.

Aspect 42: The method of Aspect 40, wherein the insoluble carrier polypeptide comprises a TAF12 polypeptide.

Aspect 43: The method of Aspect 40, wherein the insoluble carrier polypeptide comprises a trpΔLE polypeptide, a ketosteroid isomerase (KSI) polypeptide, a β-galactosidase polypeptide, a PagP polypeptide, a truncated E. coli PurF F4 fragment polypeptide, a Pseudomonas aeruginosa PaP3.30 polypeptide, a histone fold domain of the human transcription factor TAF12 (TAF12-HFD) polypeptide, a cleavable self-aggregating tag INTEIN-ELK16, a E. coli maltose-binding protein, an E. coli RNAse II polypeptide, an E. coli alkaline phosphatase polypeptide, an E. coli phospholipase A polypeptide, an E. coli β-lactamase polypeptide, a Salmonella typhimurium MalK protein, a Clostridium thermocellum endoglucanase D polypeptide, a Bacillus thuringiensis subsp. aizawai IPL7 insecticidal proteins, a human procathepsin B polypeptide, a porcine interferon-γ polypeptide, a T5 DNA polymerase polypeptide, and an E. coli thioredoxin polypeptide.

Aspect 44: The method of any one of Aspects 40-43, wherein the temperature of step c) is greater than 60° C., greater than 70° C., greater than 80° C., or greater than 90° C.

Aspect 45: The method of any one of Aspects 40-44, wherein the temperature of step c) is less than 100° C., less than 95° C., less than 90° C., or less than 85° C.

Aspect 46: The method of any one of Aspects 40-45, wherein the pH in step c) is below 3.0.

Aspect 47: The method of any one of Aspects 40-45, wherein the pH in step c) is from 2.6 to 2.8.

Aspect 48: The method of any one of Aspects 40-47, wherein the acid is a strong acid optionally selected from hydrochloric acid and sulfuric acid.

Aspect 49: The method of any one of Aspects 40-47, wherein the acid is a weak acid.

Aspect 50: The method of Aspect 49, wherein the weak acid is acetic acid.

Aspect 51: The method of Aspect 50, wherein the acetic acid concentration is at least two percent by weight, at least three percent by weight, at least four percent by weight, or at least five percent by weight.

Aspect 52: The method of Aspect 50 or Aspect 51, wherein the acetic acid concentration is less than fifty percent by weight, less than forty-five percent by weight, less than forty percent by weight, less than thirty-five percent by weight, or less than thirty percent by weight.

Aspect 53: The method of any one of Aspects 40-52, wherein the insoluble carrier polypeptide is solubilized by the incubating of step c).

Aspect 54: The method of any one of Aspects 40-52, wherein the insoluble carrier polypeptide is not solubilized by the incubating of step c).

Aspect 55. The method of any one of Aspects 40-54, wherein the fusion polypeptide comprises three or more oligopeptides, four or more oligopeptides, five or more oligopeptides, six or more oligopeptides, eight or more oligopeptides, ten or more oligopeptides, fifteen or more oligopeptides, twenty or more oligopeptides.

Aspect 56: The method of any one of Aspects 40-55, wherein the two or more oligopeptides are different.

Aspect 57: The method of any one of Aspects 40-56, wherein all the oligopeptides are operably linked to the N-terminus of the insoluble carrier polypeptide or all the oligopeptides are operably linked to the C-terminus of the insoluble carrier polypeptide.

Aspect 58: The method of any one of Aspects 40-56, wherein at least one oligopeptide is operably linked to the N-terminus of the insoluble carrier polypeptide and at least one oligopeptide is operably linked to the C-terminus of the insoluble carrier polypeptide.

Aspect 59. The method of Aspect 58, wherein the fusion polypeptide comprises two or more oligopeptides, three or more oligopeptides, four or more oligopeptides, five or more oligopeptides, six or more oligopeptides, eight or more oligopeptides, ten or more oligopeptides operably linked to the N-terminus of the insoluble carrier polypeptide and/or two or more oligopeptides, three or more oligopeptides, four or more oligopeptides, five or more oligopeptides, six or more oligopeptides, eight or more oligopeptides, ten or more oligopeptides operably linked to the C-terminus of the insoluble carrier polypeptide.

Aspect 60: The method of any one of Aspects 40-59, wherein the peptide bond comprises (i) a methionine and the sequence-specific chemical cleavage is with cyanogen bromide, (ii) a tryptophan and the sequence-specific chemical cleavage is with BNPS-skatole, (iii) an aspartic acid-proline (Asp-Pro) bond and the sequence-specific chemical cleavage is with formic acid, (iv) an asparagine-glycine bond and the sequence-specific chemical cleavage is with hydroxylamine, or (v) a cysteine and the sequence-specific chemical cleavage is with NTCB.

Aspect 61: The method of any one of Aspects 40-60, wherein the peptide bond comprises an Asp-Pro bond and the sequence-specific chemical cleavage is acid cleavage.

Aspect 62: The method of any one of Aspects 40-61, wherein the oligopeptides are operably linked by the peptide bond and are released from each other when the oligopeptides are released from the insoluble carrier polypeptide.

Aspect 63: The method of any one of Aspects 40-62, wherein the oligopeptides are operably linked by a different peptide bond and are released from each other by sequence-specific chemical cleavage of the different peptide bond after the oligopeptides are released from the insoluble carrier polypeptide.

Aspect 64: The method of Aspect 63, wherein the different peptide bond comprises (i) a methionine and the sequence-specific chemical cleavage is with cyanogen bromide, (ii) a tryptophan and the sequence-specific chemical cleavage is with BNPS-skatole, (iii) an aspartic acid-proline (Asp-Pro) bond and the sequence-specific chemical cleavage is with formic acid, (iv) an asparagine-glycine bond and the sequence-specific chemical cleavage is with hydroxylamine, or (v) a cysteine and the sequence-specific chemical cleavage is with NTCB.

Aspect 65: The method of Aspect 64, wherein the different peptide bond comprises an Asp-Pro bond and the sequence-specific chemical cleavage is acid cleavage.

Aspect 66: The method of any one of Aspects 40-65, wherein the oligopeptides are at least four amino acids long, at least five amino acids long, at least six amino acids long, at least seven amino acids long, at least eight amino acids long, at least nine amino acids long, at least ten amino acids long, at least fifteen amino acids long, at least twenty amino acids long, or at least twenty-five amino acids long.

Aspect 67: The method of any one of Aspects 40-66, wherein the oligopeptides are less than fifty amino acids long, less than forty-five amino acids long, less than forty amino acids long, less than thirty-five amino acids long, less than thirty amino acids long, less than twenty-five amino acids long, or less than twenty amino acids long.

Aspect 68: The method of any one of Aspects 40-65, wherein the oligopeptides are between four amino acids long and fifty amino acids long, between six amino acids long and forty amino acids long, between six amino acids long and thirty amino acids long, or between eight amino acids long and twenty-five amino acids long.

Aspect 69: The method of any one of Aspect 40-68, wherein the fusion peptide is expressed in bacteria or yeast.

Aspect 70: The method of Aspect 69, wherein the bacteria is E. coli or Vibrio natriegens

Aspect 71: A method of purifying inclusion bodies comprising a fusion protein comprising
a) expressing a fusion protein comprising an oligopeptide operably linked to an insoluble carrier polypeptide, which forms inclusion bodies in a cell, wherein the operable linkage is a chemically cleavable amino acid sequence,
b) lysing the cell to form a cell lysate,
c) centrifuging the cell lysate to form a pellet,
d) washing the pellet in a surfactant buffer comprising a nonionic surfactant at least once, at least twice, or at least three times,
e) washing the pellet in a salt buffer comprising at least 0.5M NaCl at least once, at least twice, or at least three times, and
f) washing the pellet in water at least once, at least twice, or at least three times, and thereby producing purified inclusion bodies.

Aspect 72: The method of Aspect 71, wherein the insoluble carrier polypeptide comprises a TAF12 polypeptide.

Aspect 73: The method of Aspect 71, wherein the insoluble carrier polypeptide comprises an onconase polypeptide.

Aspect 74: The method of Aspect 71, wherein the insoluble carrier polypeptide comprises a trpΔLE polypeptide, a ketosteroid isomerase (KSI) polypeptide, a β-galactosidase polypeptide, a PagP polypeptide, a truncated E. coli PurF F4 fragment polypeptide, a Pseudomonas aeruginosa PaP3.30 polypeptide, a histone fold domain of the human transcription factor TAF12 (TAF12-HFD) polypeptide, a cleavable self-aggregating tag INTEIN-ELK16, a E. coli maltose-binding protein, an E. coli RNAse II polypeptide, an E. coli alkaline phosphatase polypeptide, an E. coli phospholipase A polypeptide, an E. coli β-lactamase polypeptide, a Salmonella typhimurium MalK protein, a Clostridium thermocellum endoglucanase D polypeptide, a Bacillus thuringiensis subsp. aizawai IPL7 insecticidal proteins, a human procathepsin B polypeptide, a porcine interferon-γ polypeptide, a T5 DNA polymerase polypeptide, and an E. coli thioredoxin polypeptide.

Aspect 75: The method of any one of Aspects 71-74, wherein the salt buffer is at least 0.6M NaCl, at least 0.7M NaCl, or at least 0.75M NaCl.

Aspect 76. The method of any one of Aspects 71-75, wherein the fusion polypeptide comprises three or more oligopeptides, four or more oligopeptides, five or more oligopeptides, six or more oligopeptides, eight or more oligopeptides, ten or more oligopeptides, fifteen or more oligopeptides, twenty or more oligopeptides.

Aspect 77: The method of any one of Aspects 71-76, wherein the two or more oligopeptides are different.

Aspect 78: The method of any one of Aspects 71-77, wherein all the oligopeptides are operably linked to the N-terminus of the insoluble carrier polypeptide or all the oligopeptides are operably linked to the C-terminus of the insoluble carrier polypeptide.

Aspect 79: The method of any one of Aspects 71-77, wherein at least one oligopeptide is operably linked to the N-terminus of the insoluble carrier polypeptide and at least one oligopeptide is operably linked to the C-terminus of the insoluble carrier polypeptide.

Aspect 80. The method of Aspect 79, wherein the fusion polypeptide comprises two or more oligopeptides, three or more oligopeptides, four or more oligopeptides, five or more oligopeptides, six or more oligopeptides, eight or more oligopeptides, ten or more oligopeptides operably linked to the N-terminus of the insoluble carrier polypeptide and/or two or more oligopeptides, three or more oligopeptides, four or more oligopeptides, five or more oligopeptides, six or more oligopeptides, eight or more oligopeptides, ten or more oligopeptides operably linked to the C-terminus of the insoluble carrier polypeptide.

Aspect 81: The method of any one of Aspects 71-80, wherein the peptide bond comprises (i) a methionine and the sequence-specific chemical cleavage is with cyanogen bromide, (ii) a tryptophan and the sequence-specific chemical cleavage is with BNPS-skatole, (iii) an aspartic acid-proline (Asp-Pro) bond and the sequence-specific chemical cleavage is with formic acid, (iv) an asparagine-glycine bond and the sequence-specific chemical cleavage is with hydroxylamine, or (v) a cysteine and the sequence-specific chemical cleavage is with NTCB.

Aspect 82: The method of any one of Aspects 71-80, wherein the peptide bond comprises an Asp-Pro bond and the sequence-specific chemical cleavage is acid cleavage.

Aspect 83: The method of any one of Aspects 71-82, wherein the oligopeptides are operably linked by the peptide bond and are released from each other when the oligopeptides are released from the insoluble carrier polypeptide.

Aspect 84: The method of any one of Aspects 71-82, wherein the oligopeptides are operably linked by a different peptide bond and are released from each other by sequence-specific chemical cleavage of the different peptide bond after the oligopeptides are released from the insoluble carrier polypeptide.

Aspect 85: The method of Aspect 84, wherein the different peptide bond comprises (i) a methionine and the sequence-specific chemical cleavage is with cyanogen bromide, (ii) a tryptophan and the sequence-specific chemical cleavage is with BNPS-skatole, (iii) an aspartic acid-proline (Asp-Pro) bond and the sequence-specific chemical cleavage is with formic acid, (iv) an asparagine-glycine bond and the sequence-specific chemical cleavage is with hydroxylamine, or (v) a cysteine and the sequence-specific chemical cleavage is with NTCB.

Aspect 86: The method of Aspect 84, wherein the different peptide bond comprises an Asp-Pro bond and the sequence-specific chemical cleavage is with acetic acid.

Aspect 87: The method of any one of Aspects 71-86, wherein the oligopeptides are at least four amino acids long, at least five amino acids long, at least six amino acids long, at least seven amino acids long, at least eight amino acids long, at least nine amino acids long, at least ten amino acids long, at least fifteen amino acids long, at least twenty amino acids long, or at least twenty-five amino acids long.

Aspect 88: The method of any one of Aspects 71-87, wherein the oligopeptides are less than fifty amino acids long, less than forty-five amino acids long, less than forty amino acids long, less than thirty-five amino acids long, less than thirty amino acids long, less than twenty-five amino acids long, or less than twenty amino acids long.

Aspect 89: The method of any one of Aspects 71-86, wherein the oligopeptides are between four amino acids long and fifty amino acids long, between six amino acids long and forty amino acids long, between six amino acids long and thirty amino acids long, or between eight amino acids long and twenty-five amino acids long.

Aspect 90: The method of any one of Aspect 71-89, wherein the fusion peptide is expressed in bacteria or yeast.

Aspect 91: The method of Aspect 90, wherein the bacteria is E. coli or Vibrio natriegens

Aspect 92: A method of producing a fusion polypeptide comprising expressing the fusion polypeptide comprising an oligopeptide operably linked to the C-terminus of an onconase polypeptide, wherein the onconase polypeptide comprises one or more amino acid substitutions in the 11 N-terminal amino acids as compared to SEQ ID NO: 1, wherein the onconase-oligopeptide fusion protein comprising the onconase polypeptide is expressed at a higher level than a fusion protein comprising an onconase of SEQ ID NO: 1 when expressed under the same conditions.

Aspect 93: The method of Aspect 92, wherein the fusion polypeptide comprises three or more oligopeptides, four or more oligopeptides, five or more oligopeptides, six or more oligopeptides, eight or more oligopeptides, ten or more oligopeptides, fifteen or more oligopeptides, twenty or more oligopeptides.

Aspect 94: The method of Aspect 92 or Aspect 93, wherein the two or more oligopeptides are different.

Aspect 95: The method of any one of Aspects 92-94, wherein all the oligopeptides are operably linked to the N-terminus of the onconase polypeptide or all the oligopeptides are operably linked to the C-terminus of the onconase polypeptide.

Aspect 96: The method of any one of Aspects 92-94, wherein at least one oligopeptide is operably linked to the N-terminus of the onconase polypeptide and at least one oligopeptide is operably linked to the C-terminus of the onconase polypeptide.

Aspect 97. The method of Aspect 96, wherein the fusion polypeptide comprises two or more oligopeptides, three or more oligopeptides, four or more oligopeptides, five or more oligopeptides, six or more oligopeptides, eight or more oligopeptides, ten or more oligopeptides operably linked to the N-terminus of the onconase polypeptide and/or two or more oligopeptides, three or more oligopeptides, four or more oligopeptides, five or more oligopeptides, six or more oligopeptides, eight or more oligopeptides, ten or more oligopeptides operably linked to the C-terminus of the onconase polypeptide.

Aspect 98: The method of any one of Aspects 92-97, wherein the peptide bond comprises (i) a methionine and the sequence-specific chemical cleavage is with cyanogen bromide, (ii) a tryptophan and the sequence-specific chemical cleavage is with BNPS-skatole, (iii) an aspartic acid-proline (Asp-Pro) bond and the sequence-specific chemical cleavage is with formic acid, (iv) an asparagine-glycine bond and the sequence-specific chemical cleavage is with hydroxylamine, or (v) a cysteine and the sequence-specific chemical cleavage is with NTCB.

Aspect 99: The method of any one of Aspects 92-97, wherein the peptide bond comprises an Asp-Pro bond and the sequence-specific chemical cleavage is with acetic acid.

Aspect 100: The method of any one of Aspects 92-99, wherein the oligopeptides are operably linked by the peptide bond and are released from each other when the oligopeptides are released from the onconase.

Aspect 101: The method of any one of Aspects 92-99, wherein the oligopeptides are operably linked by a different peptide bond and are released from each other by sequence-specific chemical cleavage of the different peptide bond after the oligopeptides are released from the onconase.

Aspect 102: The method of Aspect 101, wherein the different peptide bond comprises (i) a methionine and the sequence-specific chemical cleavage is with cyanogen bromide, (ii) a tryptophan and the sequence-specific chemical cleavage is with BNPS-skatole, (iii) an aspartic acid-proline (Asp-Pro) bond and the sequence-specific chemical cleavage is with formic acid, (iv) an asparagine-glycine bond and the sequence-specific chemical cleavage is with hydroxylamine, or (v) a cysteine and the sequence-specific chemical cleavage is with NTCB.

Aspect 103: The method of Aspect 101, wherein the different peptide bond comprises an Asp-Pro bond and the sequence-specific chemical cleavage is acid cleavage.

Aspect 104: The method of any one of Aspects 92-103, wherein the oligopeptides are at least four amino acids long, at least five amino acids long, at least six amino acids long, at least seven amino acids long, at least eight amino acids long, at least nine amino acids long, at least ten amino acids long, at least fifteen amino acids long, at least twenty amino acids long, or at least twenty-five amino acids long.

Aspect 105: The method of any one of Aspects 92-104, wherein the oligopeptides are less than fifty amino acids long, less than forty-five amino acids long, less than forty amino acids long, less than thirty-five amino acids long, less than thirty amino acids long, less than twenty-five amino acids long, or less than twenty amino acids long.

Aspect 106: The method of any one of Aspects 92-103, wherein the oligopeptides are between four amino acids long and fifty amino acids long, between six amino acids long and forty amino acids long, between six amino acids long and thirty amino acids long, or between eight amino acids long and twenty-five amino acids long.

Aspect 107: The method of any one of Aspect 92-106, wherein the fusion peptide is expressed in bacteria or yeast.

Aspect 108: The method of Aspect 107, wherein the bacteria is E. coli or Vibrio natriegens

Aspect 109: An onconase polypeptide comprising one or more amino acid substitutions in the 11 N-terminal amino acids of the onconase polypeptide as compared to SEQ ID NO: 1, wherein the onconase polypeptide is expressed at a higher level than an onconase protein of SEQ ID NO: 1 when expressed under the same conditions.

Aspect 110: The onconase polypeptide of Aspect 109, wherein the onconase polypeptide comprises the amino acid sequence of one of SEQ ID NOs: 2-10 and 15-22, optionally with one, two, three, four, five or all six C-terminal histidine residues deleted as shown in FIG. 20A.

Aspect 111: A fusion polypeptide comprising an onconase polypeptide operably linked to one or more oligopeptides, wherein the operably linkage between the one or more oligopeptides and the onconase polypeptide comprise a peptide bond that is capable of sequence-specific chemical cleavage, wherein the onconase polypeptide comprises one or more amino acid substitutions in the 11 N-terminal amino acids of the onconase polypeptide as compared to SEQ ID NO: 1, wherein the onconase polypeptide is expressed at a higher level than an onconase protein of SEQ ID NO: 1 when expressed under the same conditions.

Aspect 112: The fusion polypeptide of Aspect 111, wherein the onconase polypeptide comprises the amino acid sequence of one of SEQ ID NOs: 2-10 and 15-22, optionally with one, two, three, four, five or all six C-terminal histidine residues deleted as shown in FIG. 20A.

Aspect 113. The fusion polypeptide of Aspect 111 or Aspect 112, wherein the fusion polypeptide comprises three or more oligopeptides, four or more oligopeptides, five or more oligopeptides, six or more oligopeptides, eight or more oligopeptides, ten or more oligopeptides, fifteen or more oligopeptides, twenty or more oligopeptides.

Aspect 114: The fusion polypeptide of any one of Aspects 111-113, wherein the two or more oligopeptides are different.

Aspect 115: The fusion polypeptide of any one of Aspects 111-114, wherein all the oligopeptides are operably linked to the N-terminus of the onconase polypeptide or all the oligopeptides are operably linked to the C-terminus of the onconase polypeptide.

Aspect 116: The fusion polypeptide of any one of Aspects 111-114, wherein at least one oligopeptide is operably linked to the N-terminus of the onconase polypeptide and at least one oligopeptide is operably linked to the C-terminus of the onconase polypeptide.

Aspect 117. The fusion polypeptide of Aspect 116, wherein the fusion polypeptide comprises two or more oligopeptides, three or more oligopeptides, four or more oligopeptides, five or more oligopeptides, six or more oligopeptides, eight or more oligopeptides, ten or more oligopeptides operably linked to the N-terminus of the onconase polypeptide and/or two or more oligopeptides, three or more oligopeptides, four or more oligopeptides, five or more oligopeptides, six or more oligopeptides, eight or more oligopeptides, ten or more oligopeptides operably linked to the C-terminus of the onconase polypeptide.

Aspect 118: The fusion polypeptide of any one of Aspects 111-117, wherein the peptide bond comprises (i) a methionine and the sequence-specific chemical cleavage is with cyanogen bromide, (ii) a tryptophan and the sequence-specific chemical cleavage is with BNPS-skatole, (iii) an aspartic acid-proline (Asp-Pro) bond and the sequence-specific chemical cleavage is with formic acid, (iv) an asparagine-glycine bond and the sequence-specific chemical cleavage is with hydroxylamine, or (v) a cysteine and the sequence-specific chemical cleavage is with NTCB.

Aspect 119: The fusion polypeptide of any one of Aspects 111-117, wherein the peptide bond comprises an Asp-Pro bond and the sequence-specific chemical cleavage is acid cleavage.

Aspect 120: The fusion polypeptide of any one of Aspects 111-119, wherein the oligopeptides are operably linked by the peptide bond and are capable of release from each other with the sequence-specific chemical cleavage.

Aspect 121: The fusion polypeptide of any one of Aspects 105-119, wherein the oligopeptides are operably linked by a different peptide bond and are capable of release from each other by a different sequence-specific chemical cleavage.

Aspect 122: The fusion polypeptide of Aspect 121, wherein the different peptide bond comprises (i) a methionine and the different sequence-specific chemical cleavage is with cyanogen bromide, (ii) a tryptophan and the different sequence-specific chemical cleavage is with BNPS-skatole, (iii) an aspartic acid-proline (Asp-Pro) bond and the different sequence-specific chemical cleavage is with formic acid, (iv) an asparagine-glycine bond and the different sequence-specific chemical cleavage is with hydroxylamine, or (v) a cysteine and the different sequence-specific chemical cleavage is with NTCB.

Aspect 123: The fusion polypeptide of Aspect 121, wherein the different peptide bond comprises an Asp-Pro bond and the different sequence-specific chemical cleavage is acid cleavage.

Aspect 124: The fusion polypeptide of any one of Aspects 111-123, wherein the oligopeptides are at least four amino acids long, at least five amino acids long, at least six amino acids long, at least seven amino acids long, at least eight amino acids long, at least nine amino acids long, at least ten amino acids long, at least fifteen amino acids long, at least twenty amino acids long, or at least twenty-five amino acids long.

Aspect 125: The fusion polypeptide of any one of Aspects 111-124, wherein the oligopeptides are less than fifty amino acids long, less than forty-five amino acids long, less than forty amino acids long, less than thirty-five amino acids long, less than thirty amino acids long, less than twenty-five amino acids long, or less than twenty amino acids long.

Aspect 126: The fusion polypeptide of any one of Aspects 111-125, wherein the oligopeptides are between four amino acids long and fifty amino acids long, between six amino acids long and forty amino acids long, between six amino acids long and thirty amino acids long, or between eight amino acids long and twenty-five amino acids long.

Aspect 127. An oligopeptide comprising an active amino acid sequence and an N-terminal proline, a C-terminal aspartic acid, or both the N-terminal proline and the C-terminal aspartic acid, wherein the active amino acid sequence is a miPEP and the oligopeptide regulates an miRNA or the active amino acid sequence is a peptide microbial inhibitor and the oligopeptide inhibits a microbe.

Aspect 128: A nucleic acid encoding a fusion polypeptide of any one of Aspects 22-39, the onconase of Aspect 109 or Aspect 110, or the fusion polypeptide of any one of Aspects 111-126.

Aspect 129: A cell comprising the nucleic acid of Aspect 128.

Aspect 130: The cell of Aspect 129, wherein the cell is a bacterial cell or a yeast cell.

Aspect 131: The cell of Aspect 129, wherein the bacteria is E. coli or Vibrio natriegens.

Aspect 132: The cell of Aspect 131, wherein the cell is a BL21 bacterial cell.

Aspect 133: The cell of Aspect 131, wherein the cell does not express Lon and ompT proteases.

Aspect 134: The nucleic acid of Aspect 128, wherein the nucleic acid is an isolated nucleic acid.

Aspect 135: The method of any one of Aspects 1-108, wherein the fusion polypeptide or fusion peptide is expressed in E. coli.

Aspect 136: The method of any one of Aspects 1-108 and 135, wherein the fusion polypeptide or fusion protein is expressed in cells grown in a fermentation bioreactor.

Aspect 137: The method of any one of Aspects 1-108, wherein the cleavage takes place for about 1-24 hours at a pH of about 2-3.5 and a temperature of about 70-90° C.

Aspect 138: A method of producing a fusion polypeptide comprising: expressing the fusion polypeptide comprising an oligopeptide operably linked to the C-terminus or the C-terminus of a modified TAF polypeptide, wherein modified TAF polypeptide comprises an amino acid sequence having at least 90%, at least 91%, at least 92%, at least 93%, at least 94%, at least 95%, at least 96%, at least 97%, at least 98%, at least 99%, or 100% sequence identity to SEQ ID NO: 23.

Aspect 139: The method of Aspect 138, wherein the fusion polypeptide comprises three or more oligopeptides, four or more oligopeptides, five or more oligopeptides, six or more oligopeptides, eight or more oligopeptides, ten or more oligopeptides, fifteen or more oligopeptides, twenty or more oligopeptides.

Aspect 140: The method of Aspect 138 or Aspect 139, wherein the two or more oligopeptides are different.

Aspect 141: The method of any one of Aspects 138-140, wherein all the oligopeptides are operably linked to the N-terminus of the modified TAF polypeptide or all the oligopeptides are operably linked to the C-terminus of the modified TAF polypeptide.

Aspect 142: The method of any one of Aspects 138-140, wherein at least one oligopeptide is operably linked to the N-terminus of the modified TAF polypeptide and at least one oligopeptide is operably linked to the C-terminus of the modified TAF polypeptide.

Aspect 143. The method of Aspect 142, wherein the fusion polypeptide comprises two or more oligopeptides, three or more oligopeptides, four or more oligopeptides, five or more oligopeptides, six or more oligopeptides, eight or more oligopeptides, ten or more oligopeptides operably linked to the N-terminus of the modified TAF polypeptide and/or two or more oligopeptides, three or more oligopeptides, four or more oligopeptides, five or more oligopeptides, six or more oligopeptides, eight or more oligopeptides, ten or more oligopeptides operably linked to the C-terminus of the modified TAF polypeptide.

Aspect 144: The method of any one of Aspects 138-143, wherein the peptide bond comprises (i) a methionine and the sequence-specific chemical cleavage is with cyanogen bromide, (ii) a tryptophan and the sequence-specific chemical cleavage is with BNPS-skatole, (iii) an aspartic acid-proline (Asp-Pro) bond and the sequence-specific chemical cleavage is with formic acid, (iv) an asparagine-glycine bond and the sequence-specific chemical cleavage is with hydroxylamine, or (v) a cysteine and the sequence-specific chemical cleavage is with NTCB.

Aspect 145: The method of any one of Aspects 138-143, wherein the peptide bond comprises an Asp-Pro bond and the sequence-specific chemical cleavage is with acetic acid or with sulfuric acid.

Aspect 146: The method of any one of Aspects 138-145, wherein the oligopeptides are operably linked by the peptide bond and are released from each other when the oligopeptides are released from the modified TAF polypeptide.

Aspect 147: The method of any one of Aspects 138-145, wherein the oligopeptides are operably linked by a different peptide bond and are released from each other by sequence-specific chemical cleavage of the different peptide bond after the oligopeptides are released from the modified TAF polypeptide.

Aspect 148: The method of Aspect 147, wherein the different peptide bond comprises (i) a methionine and the sequence-specific chemical cleavage is with cyanogen bromide, (ii) a tryptophan and the sequence-specific chemical cleavage is with BNPS-skatole, (iii) an aspartic acid-proline (Asp-Pro) bond and the sequence-specific chemical cleavage is with formic acid, (iv) an asparagine-glycine bond and the sequence-specific chemical cleavage is with hydroxylamine, or (v) a cysteine and the sequence-specific chemical cleavage is with NTCB.

Aspect 149: The method of Aspect 147, wherein the different peptide bond comprises an Asp-Pro bond and the sequence-specific chemical cleavage is acid cleavage.

Aspect 150: The method of any one of Aspects 138-149, wherein the oligopeptides are at least four amino acids long, at least five amino acids long, at least six amino acids long, at least seven amino acids long, at least eight amino acids long, at least nine amino acids long, at least ten amino acids long, at least fifteen amino acids long, at least twenty amino acids long, or at least twenty-five amino acids long.

Aspect 151: The method of any one of Aspects 138-150, wherein the oligopeptides are less than fifty amino acids long, less than forty-five amino acids long, less than forty amino acids long, less than thirty-five amino acids long, less than thirty amino acids long, less than twenty-five amino acids long, or less than twenty amino acids long.

Aspect 152: The method of any one of Aspects 138-149, wherein the oligopeptides are between four amino acids long and fifty amino acids long, between six amino acids long and forty amino acids long, between six amino acids long and thirty amino acids long, or between eight amino acids long and twenty-five amino acids long.

Aspect 153: The method of any one of Aspects 138-152, wherein the fusion peptide is expressed in bacteria or yeast.

Aspect 154: The method of Aspect 153, wherein the bacteria is E. coli or Vibrio natriegens

Aspect 155: A modified TAF polypeptide comprising an amino acid sequence having at least 90%, at least 91%, at least 92%, at least 93%, at least 94%, at least 95%, at least 96%, at least 97%, at least 98%, at least 99%, or 100% sequence identity to SEQ ID NO: 23.

Aspect 156: The modified TAF polypeptide of Aspect 155, wherein the modified TAF polypeptide comprises the amino acid sequence of SEQ ID NO: 23.

Aspect 157: A fusion polypeptide comprising a modified TAF polypeptide operably linked to one or more oligopeptides, wherein the operably linkage between the one or more oligopeptides and the modified TAF polypeptide comprise a peptide bond that is capable of sequence-specific chemical cleavage, wherein the modified TAF polypeptide comprises comprising an amino acid sequence having at least 90%, at least 91%, at least 92%, at least 93%, at least 94%, at least 95%, at least 96%, at least 97%, at least 98%, at least 99%, or 100% sequence identity to SEQ ID NO: 23.

Aspect 158: The fusion polypeptide of Aspect 157, wherein the modified TAF polypeptide comprises the amino acid sequence of SEQ ID NO: 23.

Aspect 159. The fusion polypeptide of Aspect 157 or Aspect 158, wherein the fusion polypeptide comprises three or more oligopeptides, four or more oligopeptides, five or more oligopeptides, six or more oligopeptides, eight or more oligopeptides, ten or more oligopeptides, fifteen or more oligopeptides, twenty or more oligopeptides.

Aspect 160: The fusion polypeptide of any one of Aspects 157-159, wherein the two or more oligopeptides are different.

Aspect 161: The fusion polypeptide of any one of Aspects 157-160, wherein all the oligopeptides are operably linked to the N-terminus of the modified TAF polypeptide or all the oligopeptides are operably linked to the C-terminus of the modified TAF polypeptide.

Aspect 162: The fusion polypeptide of any one of Aspects 157-160, wherein at least one oligopeptide is operably linked to the N-terminus of the modified TAF polypeptide and at least one oligopeptide is operably linked to the C-terminus of the modified TAF polypeptide.

Aspect 163. The fusion polypeptide of Aspect 162, wherein the fusion polypeptide comprises two or more oligopeptides, three or more oligopeptides, four or more oligopeptides, five or more oligopeptides, six or more oligopeptides, eight or more oligopeptides, ten or more oligopeptides operably linked to the N-terminus of the modified TAF polypeptide and/or two or more oligopeptides, three or more oligopeptides, four or more oligopeptides, five or more oligopeptides, six or more oligopeptides, eight or more oligopeptides, ten or more oligopeptides operably linked to the C-terminus of the modified TAF polypeptide.

Aspect 164: The fusion polypeptide of any one of Aspects 157-163, wherein the peptide bond comprises (i) a methionine and the sequence-specific chemical cleavage is with cyanogen bromide, (ii) a tryptophan and the sequence-specific chemical cleavage is with BNPS-skatole, (iii) an aspartic acid-proline (Asp-Pro) bond and the sequence-specific chemical cleavage is with formic acid, (iv) an asparagine-glycine bond and the sequence-specific chemical cleavage is with hydroxylamine, or (v) a cysteine and the sequence-specific chemical cleavage is with NTCB.

Aspect 165: The fusion polypeptide of any one of Aspects 157-163, wherein the peptide bond comprises an Asp-Pro bond and the sequence-specific chemical cleavage is acid cleavage.

Aspect 166: The fusion polypeptide of any one of Aspects 157-165, wherein the oligopeptides are operably linked by the peptide bond and are capable of release from each other with the sequence-specific chemical cleavage.

Aspect 167: The fusion polypeptide of any one of Aspects 157-165, wherein the oligopeptides are operably linked by a different peptide bond and are capable of release from each other by a different sequence-specific chemical cleavage.

Aspect 168: The fusion polypeptide of Aspect 167, wherein the different peptide bond comprises (i) a methionine and the different sequence-specific chemical cleavage is with cyanogen bromide, (ii) a tryptophan and the different sequence-specific chemical cleavage is with BNPS-skatole, (iii) an aspartic acid-proline (Asp-Pro) bond and the different sequence-specific chemical cleavage is with formic acid, (iv) an asparagine-glycine bond and the different sequence-specific chemical cleavage is with hydroxylamine, or (v) a cysteine and the different sequence-specific chemical cleavage is with NTCB.

Aspect 169: The fusion polypeptide of Aspect 167, wherein the different peptide bond comprises an Asp-Pro bond and the different sequence-specific chemical cleavage is acid cleavage.

Aspect 170: The fusion polypeptide of any one of Aspects 157-169, wherein the oligopeptides are at least four amino acids long, at least five amino acids long, at least six amino acids long, at least seven amino acids long, at least eight amino acids long, at least nine amino acids long, at least ten amino acids long, at least fifteen amino acids long, at least twenty amino acids long, or at least twenty-five amino acids long.

Aspect 171: The fusion polypeptide of any one of Aspects 157-170, wherein the oligopeptides are less than fifty amino acids long, less than forty-five amino acids long, less than forty amino acids long, less than thirty-five amino acids long, less than thirty amino acids long, less than twenty-five amino acids long, or less than twenty amino acids long.

Aspect 172: The fusion polypeptide of any one of Aspects 157-171, wherein the oligopeptides are between four amino acids long and fifty amino acids long, between six amino acids long and forty amino acids long, between six amino acids long and thirty amino acids long, or between eight amino acids long and twenty-five amino acids long.

Aspect 173: A nucleic acid encoding a fusion polypeptide of any one of Aspects 22-39, the modified RAF polypeptide of Aspect 155 or Aspect 156, or the fusion polypeptide of any one of Aspects 157-172.

Aspect 174: A cell comprising the nucleic acid of Aspect 173.

Aspect 175: The cell of Aspect 174, wherein the cell is a bacterial cell or a yeast cell.

Aspect 176: The cell of Aspect 174, wherein the bacteria is E. coli or Vibrio natriegens.

Aspect 177: The cell of Aspect 176, wherein the cell is a BL21 bacterial cell.

Aspect 178: The cell of Aspect 176, wherein the cell does not express Lon and ompT proteases.

Aspect 179: The nucleic acid of Aspect 173, wherein the nucleic acid is an isolated nucleic acid.

### BRIEF DESCRIPTION OF THE DRAWINGS

FIG. 1A is a schematic representation of the onconase-based fusion construct used for strategies for peptide bioproduction as exemplary means for production of peptides with insoluble carrier polypeptides. FIG. 1B shows the RP-HPLC chromatograms for the four pHs tested in the first experiment of Example 1 overlapped with the peaks for the peptide fraction and the protein fraction highlighted.

FIGS. 2A-2B compare the quantities of peptide and fusion protein released for four pHs tested from pH 2 to pH 5. FIG. 2A shows the quantity of peptide released by cleavage at the four pHs tested. FIG. 2B shows the quantity of fusion protein solubilized at the four pHs tested.

FIGS. 3A-3B compare the quantities of peptide and fusion protein released for seven pHs tested from pH 1.5 to pH 3. FIG. 3A shows the quantity of peptide released by cleavage at the seven pHs tested. FIG. 3B shows the quantity of fusion protein solubilized at the seven pHs tested.

FIGS. 4A-4B show increasing amounts of the acidic form of the peptide at decreasing pHs. FIG. 4A shows overlapping RP-HPLC chromatograms for each of the 5 pHs tested with the basic form on the left and the acidic form on the right. FIG. 4B shows the relative amounts of the basic form (black bar) and the acidic form (grey bar) at eight pHs tested.

FIGS. 5A-5B fusion protein constructs tested and SDS PAGE of the post expression cell lysate for each. FIG. 5A shows the five constructs tested. FIG. 5B shows an SDS PAGE with from left-to-right: molecular weight markers, onconase fusion, KSI fusion, PurF fusion, TAF12 fusion, and OmpX fusion. After cleavage and purification, the amounts of peptide were (relative to onconase as 100%): KSI fusion (120%), PurF fusion (75%), TAF12 fusion (265%), and OmpX fusion (37%).

FIGS. 6A-6C are schematic representations of different onconase-based fusion strategies for peptide bioproduction as exemplary means for production of peptides with insoluble carrier polypeptides. FIG. 6A is a schematic of a homoconcatemeric onconase fusion construct, where several identical peptide sequences are added with an additional aspartate-proline (DP) cleavage site, and with up to 10 copies of peptides (N). FIG. 6B is a schematic of a heteroconcatemeric construct, with copies of peptides of different sequences (M) linked with Asp-Pro (DP) cleavage sites fused to an onconase protein. FIG. 6C shows an onconase fusion with only one peptide sequence is at the C-terminus.

FIG. 7 depicts the SDS-PAGE analysis of the insoluble fraction of different onconase constructs with either 1 (X1) or 3 (X3) peptide copies. Peptides A, B and C are all 10 amino acids-long hydrophilic peptides but differ in their amino acid sequence. MW: Molecular Weight marker.

FIGS. 8A-8B shows cleavage from a concatemeric ONC fusion protein. FIG. 8A shows the RP-HPLC chromatogram after four hours of cleavage with the monopeptide, dipeptide and tripeptide peaks labeled. FIG. 8B shows the RP-HPLC chromatogram after sixteen hours of cleavage with the monopeptide and dipeptide peaks labeled.

FIGS. 9A-9C depict N-terminal variants of onconase and the results of comparative peptide yield studies. FIG. 9A is a schematic of the onconase constructs (Constructions A (SEQ ID NO: 2403), B (SEQ ID NO: 2404), C (SEQ ID NO: 2405), D (SEQ ID NO: 2406), E (SEQ ID NO: 2407), F (SEQ ID NO: 2408), G (SEQ ID NO: 2409), H (SEQ ID NO: 2410), and WT N-ter sequence (SEQ ID NO: 2411)). FIG. 9B shows the sequences of all N-terminus sequences exchanged to generate the onconase variant constructs. FIG. 9C shows a comparison of peptide yield obtained after chemical cleavage for onconase constructs A-H and the unmutated onconase.

FIG. 10 shows a comparison of MP18357 peptide quality produced with three different onconase variant constructs with modified N-terminus. Comparisons with the HPLC profile of the original onconase construct if SEQ ID NO:1 are shown the panel inserts (top right corner of each panel).

FIGS. 11A-11B show the results from the chemical cleavage optimization using the concatemeric onconase strategy. FIG. 11A is a schematic of the onconase concatemeric strategy used. Different peptide sizes are produced when incomplete chemical cleavage of the concatemer occurs. FIG. 11B shows the RP-HPLC profile for incomplete chemical digestion of the onconase concatemer at 40° C.

FIGS. 12A-12C depict a time course of the peptide distribution percentage as detected by RP-HPLC upon incubation with acetic acid. Chemical cleavage was carried out at 40° C. (FIG. 12A), 60° C. (FIG. 12B), or 80° C. (FIG. 12C). The Y axis represents the percentage of each species (monopeptide, dipeptide, or tripeptide).

FIG. 13 shows a comparison of peptide release over time after chemical cleavage of a concatemeric onconase construct at different temperatures.

FIGS. 14A-14D show the RP-HPLC analysis of a concatemeric construct with 3 copies of the MP18357 peptide. FIG. 14A shows the RP-HPLC profile obtained after solubilization at 60° C. using a protein concentration of 20 g/L, and with 20%, 30%, 40% and 50% of acetic acid (overlaid). FIG. 14B shows the RP-HPLC profile obtained after solubilization at 80° C. using a protein concentration of 50 g/L, and with 20%, 30%, 40% and 50% of acetic acid (overlaid). FIG. 14C shows the RP-HPLC profile obtained after solubilization at 60° C. using a protein concentration of 20 g/L, and with 20%, 30%, 40% and 50% of formic acid (overlaid). FIG. 14D shows the comparison of RP-HPLC profiles obtained after solubilization at 80° C. using a protein concentration of 50 g/L, and with 20% formic acid or 20% acetic acid (overlaid).

FIGS. 15A-15B are schematics of peptide production protocols. FIG. 15A is a schematic of a chaotropic agent-free protocol for peptide production, while FIG. 15B is a schematic workflow protocol that requires addition of a chaotropic agent for onconase solubilization.

FIG. 16 shows a comparison of peptide yield between when peptide production is performed using a chaotropic agent-free protocol or a protocol that requires addition of a chaotropic agent for onconase solubilization. The yields are expressed in mg/L of flask culture.

FIG. 17 shows the disease control percentage (%) for peptides sprayed on tomato plants infected with grey mold (Botrytis cinerea). The peptides were sprayed 24 h post infection at concentration of 0.1 g/l or 0.3 g/l.

FIG. 18A shows the design of onconase fusion constructs with 1 to 6 copies of the oligopeptide MP18913. FIG. 18B provides a table with the total number of peptides, the total number of peptides on the N-terminus, and the number of peptides on the C-terminus. FIG. 18C show SDS-PAGE analysis of fusion protein expression with 10 μl/well and from left to right showing ONC357_1x3, ONC357_2x3, ONC357_3x3, ONC357x3, ONC357x4, NEB P7717 molecular weight markers, ONC357x3, ONC357x4, ONC357x5, ONC357x6, and NEB P7717 molecular weight markers.

FIG. 19 shows a HPLC analysis of peptide release following acidic cleavage.

FIG. 20A shows an amino acid sequence comparison between SEQ ID NO: 2412 and an onconase variant comprising three lysine to alanine substitutions and deletion of six C-terminal histidines (SEQ ID NO: 2413). FIG. 20B shows an alignment of several onconase variants (SEQ ID NOS: 2414-2421).

FIGS. 21A-21B show exemplary schematics of the cells and batch cultures that result from the incorporation of the inclusion bodies into E. coli culture. FIG. 21A shows an exemplary schematic of an E. coli cell containing a DNA concatemer that includes a fusion carrier protein (also marked with an asterisk), two amino acids used repeatedly as linkers (“XY”), and peptides of interest (“PEP”) located between the linkers. FIG. 21B shows an exemplary bioreactor in the process of scaling batches of expressed peptide titers (left) and an E. coli cell (right) producing an inclusion body (dashed grey line) as a result of the activity of the incorporated DNA concatemer.

### DETAILED DESCRIPTION

Provided herein are methods of producing oligopeptides, methods of releasing an oligopeptide fused to an insoluble carrier polypeptide that forms inclusion bodies in a cell, methods of purifying inclusion bodies comprising a fusion protein, and methods of producing a fusion polypeptide. Also provided herein are fusion polypeptides comprising an insoluble carrier polypeptide (e.g., an onconase polypeptide or a TAF12 polypeptide) operably linked to two or more oligopeptides, and modified insoluble carrier polypeptides (e.g., onconase polypeptides or TAF12 polypeptides), oligopeptides comprising an active amino acid sequence.

The methods and polypeptides of the disclosure are based, at least in part, on Applicant's discovery of a bioproduction process utilizing concatemeric protein constructs comprising an insoluble carrier polypeptide (e.g., an onconase polypeptide or a TAF12 polypeptide) and two or more oligopeptides that increased peptide yield and quality of peptides over other bioproduction methods. Applicant's bioproduction process surprisingly improved the production of short, biologically active peptides, which are known in the art to be challenging to produce. In contrast to previous methods, the methods disclosed herein do not require the use of a chaotropic agent or a column-based purification step, and thus reduce the financial and technical burden of peptide bioproduction. Applicant has also developed variant insoluble carrier polypeptides (e.g., onconase polypeptides or TAF12 polypeptides) that exhibit increased expression in inclusion bodies when expressed in a host cell. Accordingly, these variant onconase polypeptide may be used as a fusion partner for increasing bioproduction of a peptide of interest operably linked to the variant onconase. Without wishing to be bound by theory, the increased accumulation of the peptide of interest in inclusion bodies allows for a higher level of the peptide to be purified from a host cell.

As used herein, an “amino acid” or “amino acid residue” refers to any naturally occurring amino acid, any non-naturally occurring amino acid, any modified amino acid, including derivatized amino acids, or any amino acid mimetic known in the art. The amino acid may be referred by both their common three letter abbreviation and single letter abbreviation.

As used herein, the term “peptide” refers to any peptide structure comprising or consisting of two or more amino acids, including chemical modifications and derivatives of amino acids. In some embodiments, the peptide is a short peptide (e.g., 4 to 50 amino acids in length). In some embodiments, a peptide is an oligopeptide released upon cleavage of a polypeptide.

As used herein, the term “purified” molecule refers to biological or synthetic molecules that are removed from their natural environment and are isolated or separated and are free from other components with which they are naturally associated.

The term “isolated” in reference to a molecule including a nucleic acid, construct, vector, etc., may refer to a molecule that is not found in nature and/or is present in a context in which it is not found in nature. The term “isolated” may also refer to a molecule that has undergone at least one step towards being isolated or concentrated or enriched from a more complex solution or source. The term “isolated,” however, is in no way intended to limit the molecule to a particular location or state. For example, an isolated nucleic acid molecule includes the nucleic acid molecule introduced into the genome of a cell in a position where it is not found in nature or when it is resident in progeny of cells into which the nucleic acid molecule has been introduced into its genome in a position where it is not found in nature.

The term “sequence identity” refers to the degree of similarity between two nucleic acid sequences, or two amino acid sequences, is expressed in terms of the similarity between the sequences, and otherwise referred to as sequence identity. Sequence identity is frequently measured in terms of percentage identity (or similarity or homology); the higher the percentage, the more similar the two sequences are.

As used herein, a “concatemeric polypeptide”, or “concatemer,” refers to a polypeptide that includes multiple copies of a given unit (e.g., an oligopeptide) as tandem repeats. In some embodiments a concatemeric polypeptide is a homoconcatemer (each oligopeptide unit is the same). In some embodiments, a concatemeric polypeptide is a heteroconcatemer (there are more than one different oligopeptide). In some embodiments, the concatemeric polypeptide comprises a linker between the units.

As used herein, “active amino acid sequence,” “biologically active amino acid sequence,” and “active sequence” refer to an amino acid sequence capable of inducing a biological effect in living cells and/or organisms that are exposed to said amino acid sequence. In some embodiments, the active amino acid sequence has microbe inhibiting activity. In some embodiments, the active amino acid sequence confers a benefit to a plant.

As used herein “onconase variant” is an onconase polypeptide that comprises one or more amino acid insertions, deletions, or substitutions compared to the amino acid sequence set forth in SEQ ID NO:1.

### I. Methods of Producing Oligopeptides

Some aspects of the present disclosure provide for methods of producing oligopeptides. In some embodiments, the methods for producing oligopeptides comprise expressing a fusion polypeptide comprising an insoluble carrier polypeptide (e.g., an onconase polypeptide or a TAF12 polypeptide) operably linked to two or more oligopeptides by a peptide bond, and releasing the releasing the two or more oligopeptides from the insoluble carrier polypeptide (e.g., an onconase polypeptide or a TAF12 polypeptide) by sequence-specific chemical cleavage of the peptide bond.

### Fusion Proteins

In some embodiments, the fusion polypeptide comprising an insoluble carrier polypeptide (e.g., an onconase polypeptide or a TAF12 polypeptide) is operably linked to two or more oligopeptides. In some embodiments, the method comprises expressing a fusion polypeptide comprising an insoluble carrier polypeptide (e.g., an onconase polypeptide or a TAF12 polypeptide) and two or more polypeptides. For example, the fusion polypeptide comprises an insoluble carrier polypeptide (e.g., an onconase polypeptide or a TAF12 polypeptide) operably linked to three or more oligopeptides, four or more oligopeptides, five or more oligopeptides, six or more oligopeptides, eight or more oligopeptides, ten or more oligopeptides, fifteen or more oligopeptides, twenty or more oligopeptides. In some embodiments, the fusion polypeptide comprises two or more oligopeptides that have the same amino acid sequence. In some embodiments, the fusion polypeptide comprises two or more oligopeptides that have different amino acid sequences. In some embodiments, the fusion polypeptide comprises two or more oligopeptides that have the same amino acid sequence and two or more oligopeptides comprising different amino acid sequences.

In some embodiments, the fusion polypeptide comprises an insoluble carrier polypeptide (e.g., an onconase polypeptide or a TAF12 polypeptide) and two or more oligopeptides operably linked to the N-terminus and/or the C-terminus of the onconase protein. For instance, the fusion polypeptide may include two or more oligopeptides operably linked to each of the N-terminus and the C-terminus of the insoluble carrier polypeptide (e.g., an onconase polypeptide or a TAF12 polypeptide). In some embodiments, the fusion polypeptide comprises a total of 2, 3, 4, 5, 6, 7, 8, 9, 10, 15, 20, 25, or more oligopeptides operably linked to the N-terminus and/or the C-terminus of the onconase protein. In some embodiments, the number of the oligopeptides operably linked to each terminus of the onconase may be the same, or different.

In some embodiments, the fusion polypeptide comprises an insoluble carrier polypeptide (e.g., an onconase polypeptide or a TAF12 polypeptide), and no oligopeptides operably linked to the N-terminus of the insoluble carrier polypeptide (e.g., an onconase polypeptide or a TAF12 polypeptide) and two or more oligopeptides operably linked to the C-terminus. In some embodiments, the fusion polypeptide comprises an insoluble carrier polypeptide (e.g., an onconase polypeptide or a TAF12 polypeptide), and no oligopeptides operably linked to the N-terminus of the insoluble carrier polypeptide (e.g., an onconase polypeptide or a TAF12 polypeptide) and four, five or six oligopeptides operably linked to the C-terminus. In some embodiments, the fusion polypeptide comprises an insoluble carrier polypeptide (e.g., an onconase polypeptide or a TAF12 polypeptide), and two or more oligopeptides operably linked to the N-terminus of the insoluble carrier polypeptide (e.g., an onconase polypeptide or a TAF12 polypeptide) and three oligopeptides operably linked to the C-terminus. In some embodiments, the fusion polypeptide comprises an insoluble carrier polypeptide (e.g., an onconase polypeptide or a TAF12 polypeptide), and one, two or three oligopeptides operably linked to the N-terminus of the insoluble carrier polypeptide (e.g., an onconase polypeptide or a TAF12 polypeptide) and three oligopeptides operably linked to the C-terminus. In some embodiments, one or more of the oligopeptides operably linked to the N-terminus of the onconase may have the same amino acid sequence as one or more of the oligopeptides operably linked to the C-terminus of the insoluble carrier polypeptide (e.g., an onconase polypeptide or a TAF12 polypeptide). In some embodiments, one or more of the oligopeptides operably linked to the N-terminus of the onconase have a different amino acid sequence than one or more of the oligopeptides operably linked to the C-terminus of the onconase. In some embodiments, the fusion polypeptide comprises an insoluble carrier polypeptide (e.g., an onconase polypeptide or a TAF12 polypeptide) and two or more oligopeptides linked to the C-terminus of the onconase. In some embodiments, two or more oligopeptides linked to the C-terminus of the onconase have the same amino acid sequence. In some embodiments, two or more oligopeptides linked to the C-terminus of the onconase have a different amino acid sequence. In some embodiments, the fusion polypeptide comprises an insoluble carrier polypeptide (e.g., an onconase polypeptide or a TAF12 polypeptide) and two or more oligopeptides are linked to the N-terminus of the onconase. In some embodiments, two or more oligopeptides linked to the N-terminus of the onconase have the same amino acid sequence. In some embodiments, two or more oligopeptides linked to the N-terminus of the onconase have a different amino acid sequence. In some instances, the insoluble carrier polypeptide (e.g., an onconase polypeptide or a TAF12 polypeptide) is separated from the two or more oligopeptides by a peptide bond. In some embodiments, the two or more oligopeptides are separated themselves by a peptide bond. In some embodiments, the peptide bond includes a cleavable bond.

The cleavable bond may be cleaved by enzymatic or chemical agents. In some embodiments, the cleavable bond is a peptide bond. Cleavage of the peptide bond may result in separation of the insoluble carrier polypeptide (e.g., an onconase polypeptide or a TAF12 polypeptide) from the oligopeptides and/or of the oligopeptides from each other. Any cleavable peptide bond cleavable by any methods or agents in the art may be used with the present methods. Proteolytic enzymes and their respective cleavage site specificities are well known in the art. Examples of enzymes useful for cleaving the peptide linker include, but are not limited to Arg-C proteinase, Asp-N endopeptidase, chymotrypsin, clostripain, enterokinase, Factor Xa, glutamyl endopeptidase, Granzyme B, Achromobacter proteinase I, pepsin, proline endopeptidase, proteinase K, Staphylococcal peptidase I, thermolysin, thrombin, trypsin, TEV protease, HRV3C, and members of the Caspase family of proteolytic enzymes (e.g. Caspases 1-10). Chemical cleavage agents are also well known in the art and can results in cleavage of a polypeptide at a peptide bond between specific pairs of amino acids. Examples of chemical cleavage reagents include cyanogen bromide (cleaves after methionine residues leaving a C-terminal homoserine lactone in place of the methionine), N-chloro succinimide, iodobenzoic acid or BNPS-skatole [2-(2-nitrophenylsulfenyl)-3-methylindole](each of which cleaves tryptophan residues leaving a C-terminal tryptophan (with lactone)), dilute acids (which cleave at aspartyl-prolyl bonds leaving a C-terminal aspartate and an N-terminal proline), and hydroxylamine (which cleaves at asparagine-glycine bonds at pH 9.0 leaving a C-terminal asparagine and an N-terminal glycine). Additional chemical cleavage agents are described in Gavit, P. and Better, M., J. Biotechnol., 79:127-136 (2000); Szoka et al., DNA, 5(1):11-20 (1986); and Walker, J. M., The Proteomics Protocols Handbook, 2005, Humana Press, Totowa, N.J.)). In some embodiments, the peptide cleavable bonds between the peptides are cleavable by the same reagent as the peptide cleavable bonds between the peptide and the onconase. In some embodiments, the peptide cleavable bonds between the peptides are cleavable by a different reagent from the peptide cleavable bonds between the peptide and the onconase.

In some embodiments, the cleavage is at a specific amino acid sequence. In some embodiments, the peptide bond includes (i) a methionine and the sequence-specific chemical cleavage is with cyanogen bromide, (ii) a tryptophan and the sequence-specific chemical cleavage is with BNPS-skatole, (iii) an aspartic acid-proline (Asp-Pro) bond and the sequence-specific chemical cleavage is with formic acid, acetic acid, or any diluted acid solution, (iv) an asparagine-glycine bond and the sequence-specific chemical cleavage is with hydroxylamine, or (v) a cysteine and the sequence-specific chemical cleavage is with NTCB. In some embodiments, the two or more oligopeptides are operably linked to the insoluble carrier polypeptide (e.g., an onconase polypeptide or a TAF12 polypeptide) by a peptide bond comprising an Asp-Pro bond and the sequence-specific chemical cleavage is with formic acid or acetic acid. In some embodiments, the peptides are linked to one another by a peptide bond comprising an Asp-Pro bond and the sequence-specific chemical cleavage is with formic acid or acetic acid.

The two or more oligopeptides may correspond to any desired amino acid sequence. In some instances, the oligopeptides may be small peptides (e.g., 4 to 25 amino acids in length). For example, the oligopeptides may be at least four, at least five, at least six, at least seven, at least eight, at least nine, at least ten, at least fifteen, at least twenty, or at least twenty-five amino acids long. The oligopeptides may instead be longer peptides (e.g., up to fifty amino acids in length). The oligopeptides may be less than fifty, less than forty-five, less than forty, less than thirty-five, less than thirty, less than twenty-five, or less than twenty amino acids long. In some instances, the oligopeptides are between four and fifty, between six and forty amino acids long, between six and thirty amino acids long, or between eight and twenty-five amino acids long.

In some embodiments, the one or more oligopeptide comprises the amino acid sequence of any one of SEQ ID NOs: 12-14 and 31-2401. In some embodiments, one or more oligopeptides comprise an amino acid sequence that has at least 80%, at least 85%, at least 90%, at least 91%, at least 92%, at least 93%, at least 94%, at least 95%, at least 96%, at least 97%, at least 98%, or at least 99% sequence identity with the amino acid sequence set forth in SEQ ID NO: 12. In some embodiments, one or more oligopeptides comprise an amino acid sequence that has between 80% to 85%, 85% to 90%, 90% to 95%, or 95% to 100% sequence identity with the amino acid sequence set forth in SEQ ID NO: 13. In some embodiments, the one or more oligopeptides comprise at least one amino acid sequence that has at least 80%, at least 85%, at least 90%, at least 91%, at least 92%, at least 93%, at least 94%, at least 95%, at least 96%, at least 97%, at least 98%, or at least 99% sequence identity with the amino acid sequence set forth in SEQ ID NO: 14.

In some embodiments, the fusion polypeptide comprises an insoluble carrier polypeptide (e.g., an onconase polypeptide or a TAF12 polypeptide) linked to two or more micropeptides (miPEPs). miPEPs are short peptides (7-44 amino acids long) defined by one or several short open reading frames located in the pri-microRNA sequence of a specific miRNA member. Without wishing to be bound by theory, miPEPs target the pri-microRNA of their encoding miRNA to either upregulate or downregulate transcription. miPEPs may target miRNAs involved various processes, such as miRNAs involved in immunity and susceptibility to a pathogen (e.g., a microbe), organogenesis, response to stress, embryonic development, etc. In some embodiments, each of the two or more oligopeptides comprise a miPEP sequence that regulates a miRNA. In some embodiments, the two or more oligopeptides comprise a miPEP sequence that regulates one or more miRNA families. In some embodiments, each of the two or more oligopeptides comprise a miPEP sequence that regulates one or more members of a particular miRNA family. In some embodiments, each of the two or more oligopeptides comprise a miPEP sequence that regulates a plant miRNA, a fungal miRNA, or a metazoan miRNA. In some embodiments, each of the two or more oligopeptides comprise a miPEP sequence that regulates a plant miRNA. Exemplary plant microRNA families that may be regulated by miPEPs include, but are not limited to, plant miRNA families miR156, miR159/319, miR160, miR162, miR164, miR166, miR167, miR168, miR169, miR171, miR172, miR390, miR393, miR394, miR295, miR396, miR397, miR398, miR408, miR403, miR437, miR444, and miR445. In some embodiments, each of the two or more oligopeptides comprise a miPEP sequence that has microbe inhibiting activity.

In some embodiments, the oligopeptides comprise any other peptide of interest. In some embodiments, the oligopeptides comprise a polypeptide that is toxic to a host cell. In some embodiments, the oligopeptides comprise a therapeutic polypeptide, a toxin, a cytokine, a peptide hormone, a clotting factor, an immunogenic peptide, an allergen, an antimicrobial peptide, a ligand binding domain, or any combination thereof. In some embodiments, the immunogenic peptide is a peptide that can induce an immune response to a polypeptide comprising said peptide. In some embodiments, the one or more oligopeptides comprise a peptide fragment of a larger polypeptide. For example, the one or more oligopeptides may comprise a peptide fragment of a receptor, an enzyme, an adhesion molecule or a structural protein.

In some embodiments, the oligopeptides comprise an active amino acid sequence and a tag at the N-terminus and/or C-terminus. In some embodiments, the N-terminus and/or C-terminus residues may correspond to a cleavage tag produced after release of the oligopeptides. For instance, the residue may be part of a peptide bond that is cleaved by chemical (e.g. acidic cleavage), or enzymatic reactions. In some embodiments, the oligopeptide comprises an active amino acid sequence and an N-terminal proline, a C-terminal aspartic acid, or both the N-terminal proline and the C-terminal aspartic acid. In some embodiments, the active amino acid sequence has antimicrobial activity.

The fusion polypeptide may also include a linker sequence between the two or more oligopeptides and/or between the insoluble carrier polypeptide (e.g., an onconase polypeptide or a TAF12 polypeptide) and the oligopeptides. The linker sequence may function as a spacer peptide to separate the two or more oligopeptides and/or the insoluble carrier polypeptide (e.g., an onconase polypeptide or a TAF12 polypeptide) and oligopeptides. Any number or linker sequences known in the art may be used. In some embodiments, the linker is about 1 to 50 amino acids in length. In some embodiments, the linker is about 4 to 8 amino acids in length. In some embodiments, the linker sequence is a flexible amino acid sequence. In some embodiments, the flexible linker comprises glycine, serine, or threonine residues, or any combination thereof. In some embodiments, the flexible linker sequence comprises a (GGGGS)ₙ (SEQ ID NO: 2402) or a (G)ₙ sequence, wherein n is an integer. In some embodiments, the flexible linker comprises a GGG, GSGS (SEQ ID NO: 30), GSGSGGT (SEQ ID NO: 31), GGSGTG (SEQ ID NO: 27) or GTGSGTG (SEQ ID NO: 32) amino acid sequence. In some embodiments, the linker sequence is a rigid linker sequence. In some embodiments, the rigid linker sequence comprises a (EAAAK)ₙ (SEQ ID NO: 33) or (XP)ₙ sequence, wherein n is an integer and X is any amino acid. In some embodiments, the linker sequence comprises a flexible linker sequence and a rigid linker sequence. The linker sequence may also provide a cleavable peptide bond by incorporation of a cleavable linker sequence that comprises an amino acid sequence that directs the sequence-specific cleavage of the fusion polypeptide. In some embodiments, the linker sequence may include a sequence for protease or chemical cleavage. In some embodiments, the fusion polypeptide comprises linker sequence comprising the amino acid sequence of SEQ ID NO: 11. In some embodiments, the fusion polypeptide comprises linker sequence comprising the amino acid sequence of SEQ ID NO: 11. In some embodiments, the fusion polypeptide comprises linker sequence comprising the amino acid sequence of SEQ ID NO: 11. In some embodiments, the linker comprises an Asp-Pro sequence.

In some embodiments, the fusion polypeptide comprises linker sequence separating the two or more oligopeptides and/or a linker sequence separating the insoluble carrier polypeptide (e.g., an onconase polypeptide or a TAF12 polypeptide) from the two or more oligopeptides. In some embodiments, the linker sequence separating the two or more oligopeptides has the same sequence as the linker separating the onconase sequence from the oligopeptides. In some embodiments, the linker sequence between the oligopeptides is different than the linker sequence separating the insoluble carrier polypeptide (e.g., an onconase polypeptide or a TAF12 polypeptide) from the two or more oligopeptides. In some embodiments, the linker sequence provides a cleavable peptide-bond. In some embodiments, the linker sequence comprises an amino acid sequence for sequence-specific cleavage of the linker. In some embodiment, the sequence-specific cleavage is acidic cleavage. In some embodiments, the fusion polypeptide comprises linker sequence comprising the amino acid sequence of SEQ ID NO: 11 separating the two or more oligopeptides and/or separating the insoluble carrier polypeptide (e.g., an onconase polypeptide or a TAF12 polypeptide) from the two or more oligopeptides. In some embodiments, the fusion polypeptide comprises a linker sequence comprising an Asp-Pro linker separating the two or more oligopeptides and/or separating the insoluble carrier polypeptide (e.g., an onconase polypeptide or a TAF12 polypeptide) from the two or more oligopeptides.

In some embodiments, the fusion polypeptide further comprises one or more amino acid sequence tags. In some embodiments, the amino acid sequence tag does not affect the solubility of the onconase fusion protein. In some embodiments, the amino acid sequence tag has a neutral pI. In some embodiments, the one or more amino acid sequence tags are purification tag peptides. Such tag peptides may include, but are not limited to, glutathione-S-transferase (GST), polyhistidine, maltose binding protein (MBP), avidin, biotin, streptavidin, histidine (His) tags (e.g. His-6X tag), V5 tags, FLAG tags, influenza hemagglutinin (HA) tags, Myc tags, VSV-G tags, thioredoxin (Trx) tags, and ligands for cellular receptor (e.g., insulin receptor ligands). In some embodiments, the one or more amino acid sequence tags are reporter tag peptide sequences. Examples of reporter tag peptide sequences include without limitation horseradish peroxidase (HRP), chloramphenicol acetyltransferase (CAT), beta-galactosidase, beta-glucuronidase, luciferase, and fluorescent proteins (e.g., GFP, CFP, YFP, BFP, etc.).

In some embodiments, the one or more amino acid sequence tags are operably linked to the N-terminus and/or C-terminus of the insoluble carrier polypeptide (e.g., an onconase polypeptide or a TAF12 polypeptide) or the two or more oligopeptides. In some embodiments, the one or more amino acid sequence tags are operably linked to the N-terminus and/or C-terminus of the insoluble carrier polypeptide (e.g., an onconase polypeptide or a TAF12 polypeptide). In some embodiments, the one or more tag peptides are operably linked to the C-terminus of the insoluble carrier polypeptide (e.g., an onconase polypeptide or a TAF12 polypeptide). In some embodiments, the one or more tag peptides are operably linked to the N-terminus of the insoluble carrier polypeptide (e.g., an onconase polypeptide or a TAF12 polypeptide). In some embodiments, the one or more amino acid sequence tags are operably linked to the N-terminus and/or C-terminus of the two or more oligopeptides. In some embodiments, the one or more tag peptides are operably linked to the N-terminus of the two or more oligopeptides. In some embodiments, the one or more tag peptides are operably linked to the C-terminus of two or more oligopeptides. In some embodiments, the one or more amino acid sequence tags are operably linked to the C-terminus of the insoluble carrier polypeptide (e.g., an onconase polypeptide or a TAF12 polypeptide) and the N-terminus of the two or more oligopeptides. In some embodiments, the one or more amino acid sequence tags are operably linked to the N-terminus of the onconase and the C-terminus of the one or more oligopeptides. In some embodiments, the one or more amino acid sequence tags are operably linked to the C-terminus of one oligopeptide and the N-terminus of another oligopeptide. In some embodiments, the amino acid sequence tag operably linked to the insoluble carrier polypeptide (e.g., an onconase polypeptide or a TAF12 polypeptide) is the same as the amino acid sequence tag operably linked to the two or more oligopeptides. In some embodiments, the amino acid sequence tag operably linked to the insoluble carrier polypeptide (e.g., an onconase polypeptide or a TAF12 polypeptide) is different than the amino acid sequence tag operably linked to the two or more oligopeptides.

Onconase is an RNAse that exhibits pH-dependent changes in solubility. The solubility of onconase is determined by its isoelectric point (pI). The pI of a protein is the pH at which the protein carries no net electrical charge. In acidic conditions, when the pH is lower than the pI, onconase is positively charged and is soluble. At neutral conditions, when the pH is around the pI, onconase is neutrally charged and forms insoluble aggregates. These insoluble aggregates can be removed from a solution by selective precipitation. The pI corresponds to the average of the acid dissociation constants, or pKa, of amino acids in a particular protein. Thus, the pI is higher when a protein comprises more Arg (pKa=12.48, Lys (pKa=10.79), Tyr (pKa=10.07), Cys (pKa=8.35), His, (pKa=6.04), Asp (pKa=3.86), and Glu (pKa=4.25) residues, and lower when the protein comprises more non-polar amino acid residues. Onconase is a highly cationic protein and has a pI higher than 9.5. In some embodiments, the onconase has a pI of around 7.5. In some embodiments, the onconase has a neutral pI. Moreover, in some embodiments, the onconase lacks any aspartate-proline and asparagine-glycine sequences, which makes it resistant to common chemical cleavage strategies, such as acidic cleavage.

In some embodiments, the fusion polypeptide comprises an onconase polypeptide comprising the amino acid sequence of SEQ ID NO: 1 operably linked to two or more oligopeptides by a peptide bond. In some embodiments, the fusion polypeptide comprises an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to SEQ ID NO: 1. In some embodiments, the fusion polypeptide comprises an onconase polypeptide comprising an amino acid sequence that has between 80% to 85%, 85% to 90%, 90% to 95%, or 95% to 100% sequence identity to SEQ ID NO: 1. In some embodiments, the onconase polypeptide is a truncated onconase polypeptide.

In some embodiments, the fusion polypeptide comprises a variant onconase polypeptide operably linked to two or more oligopeptides by a peptide bond. The variant onconase may be an onconase with mutations that decreased susceptibility to protein cleavage, increase yield, promote aggregation, or promote solubility. For example, the M23L-onconase (M23L-ONC) disclosed by Pane, et al. (Pane, K., et al. (2016) PLoS One. 11(1):e0146552, incorporated herein by reference) does not contain any internal methionine residues and is less susceptible to chemical cleavage strategies. The modifications may be one or more of amino acid substitutions, deletions, or insertions. Other amino acid substitutions, insertions or deletions may be introduced that increase stability, yield or aggregation of the onconase protein.

In some embodiments, the fusion polypeptide comprises a variant onconase comprising one or more amino acid substitutions as compared to the amino acid sequence of SEQ ID NO: 1. In some embodiments, the one or more amino acid substitutions are conservative amino acid substitutions. Conservative amino acid substitutions refer to the substitution of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. In some embodiments, the one or more amino acid substitutions are non-conservative amino acid substitutions. Non-conservative substitutions refer to substitutions between amino acids with different properties (e.g., a non-charged residue substituted by a charged residue). In some embodiments, the fusion protein comprises an onconase polypeptide comprising one or more insertions or deletions as compared to the amino acid sequence of SEQ ID NO: 1. The one or more insertions or deletions may be of any amino acid length.

In some embodiments, the fusion protein comprises an onconase polypeptide with one or more amino acid substitutions that decrease susceptibility to chemical cleavage as compared to an onconase polypeptide having the amino acid sequence of SEQ ID NO: 1. In some embodiments, the fusion protein comprises an onconase polypeptide with one or more deletions or insertions that reduce susceptibility to chemical cleavage as compared to an onconase polypeptide having the amino acid sequence of SEQ ID NO: 1.

In some embodiments, the fusion protein comprises an onconase polypeptide with one or more amino acid substitutions that increase yield or promote formation of inclusion bodies as compared to an onconase polypeptide having the amino acid sequence of SEQ ID NO: 1. In some embodiments, the one or more amino acid substitutions that increase yield or promote formation of inclusion bodies comprise one or more substitutions of cationic or polar amino acids for non-polar or neutral amino acids. In some embodiments, the onconase comprises one or more amino acid substitutions, insertions, or deletions that reduce solubility. In some embodiments, the one or more amino acid substitutions result in a variant onconase polypeptide having a decreased number of cationic or polar amino acids as compared to an onconase polypeptide having the amino acid sequence of SEQ ID NO: 1. In some embodiments, the one or more amino acid substitutions that increase yield or promote formation of inclusion bodies result in a variant onconase having a decreased number of Lys, Arg, Cys, His, Asp, Glu, and/or Tyr as compared to an onconase polypeptide having the amino acid sequence of SEQ ID NO: 1. In some embodiments, the fusion protein comprises an onconase polypeptide with one or more amino acid substitutions that result in an onconase protein having an altered pI as compared to an onconase polypeptide having the amino acid sequence of SEQ ID NO: 1. In some embodiments, the fusion protein comprises an onconase polypeptide with one or more deletions or insertions that increase yield or promote formation of inclusion bodies as compared to an onconase polypeptide having the amino acid sequence of SEQ ID NO: 1. In some embodiments, the one or more insertions or deletions that increase yield or promote formation of inclusion bodies result in a variant onconase having a decreased number of cationic or polar amino acids as compared to an onconase polypeptide having the amino acid sequence of SEQ ID NO: 1. In some embodiments, the one or more insertions or deletions that increase yield or promote formation of inclusion bodies result in a variant onconase having a decreased number of Lys, Arg, Cys, His, Asp, Glu, and/or Tyr as compared to an onconase polypeptide having the amino acid sequence of SEQ ID NO: 1. In some embodiments, the fusion protein comprises an onconase polypeptide with one or more deletions or insertions that increase yield or promote formation of inclusion bodies as compared to an onconase polypeptide having the amino acid sequence of SEQ ID NO: 1. In some embodiments, the fusion protein comprises an onconase polypeptide with one or more deletions or insertions that result in an onconase protein having an altered pI as compared to an onconase polypeptide having the amino acid sequence of SEQ ID NO: 1. In any of the above embodiments, the onconase polypeptide may comprise an amino acid sequence that has at least 80%, at least 85%, at least 90%, at least 91%, at least 92%, at least 93%, at least 94%, at least 95%, at least 96%, at least 97%, at least 98%, or at least 99% sequence identity to SEQ ID NO: 1. In some embodiments, the onconase polypeptide comprises an amino acid sequence that has between 80% to 85%, 85% to 90%, 90% to 95%, or 95% to 100% sequence identity to SEQ ID NO: 1.

In some embodiments, the fusion protein comprises an onconase polypeptide with one or more N-terminal amino acid substitutions compared to SEQ ID NO: 1. In some embodiments, the fusion protein comprises an onconase polypeptide with 1, 2, 3, 4, 5, 6, 8, 9, 10, 11, 12, 13, 14, 15, 20, 25, 30, or 35 N-terminal amino acid substitutions compared to SEQ ID NO: 1. In some embodiments, the fusion protein comprises an onconase polypeptide with 11 N-terminal amino acid substitutions compared to SEQ ID NO: 1. In some embodiments, the amino acid substitutions are within the first 50 N-terminal amino acids, within the first 40 N-terminal amino acids, within the first 30 N-terminal amino acids, within the first 20 N-terminal amino acids, or within the first 11 amino acids. In some embodiments, the amino acid substitutions are within the first 15 N-terminal amino acids, within the first 14 N-terminal amino acids, within the first 13 N-terminal amino acids, within the first 12 N-terminal amino acids, within the first 11 N-terminal amino acids, within the first 10 N-terminal amino acids, within the first 9 N-terminal amino acids, within the first 8 N-terminal amino acids, within the first 7 N-terminal amino acids, within the first 6 N-terminal amino acids, within the first 5 N-terminal amino acids, within the first 4 N-terminal amino acids, within the first 3 N-terminal amino acids, or within the first 2 N-terminal amino acids. In some embodiments, one or more N-terminal amino acid substitutions are conservative amino acid substitutions. In some embodiments, one or more N-terminal amino acid substitutions are non-conservative amino acid substitutions. In some embodiments, the fusion protein comprises an onconase polypeptide with one or more N-terminal insertions or deletions compared to SEQ ID NO: 1. In some embodiments, the fusion polypeptide comprises an onconase polypeptide comprises the amino acid sequence of SEQ ID NO: 1. In some embodiments, the fusion polypeptide comprises an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to SEQ ID NO: 1. In some embodiments, the fusion polypeptide comprises an onconase polypeptide comprising an amino acid sequence that has between 80% to 85%, 85% to 90%, 90% to 95%, or 95% to 100% sequence identity to SEQ ID NO: 1.

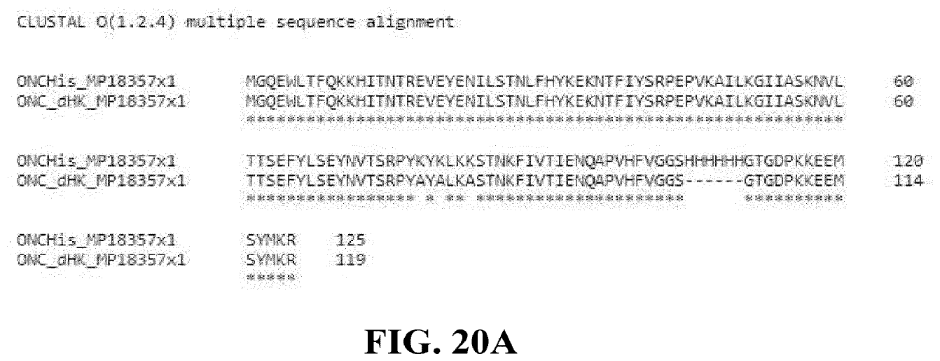

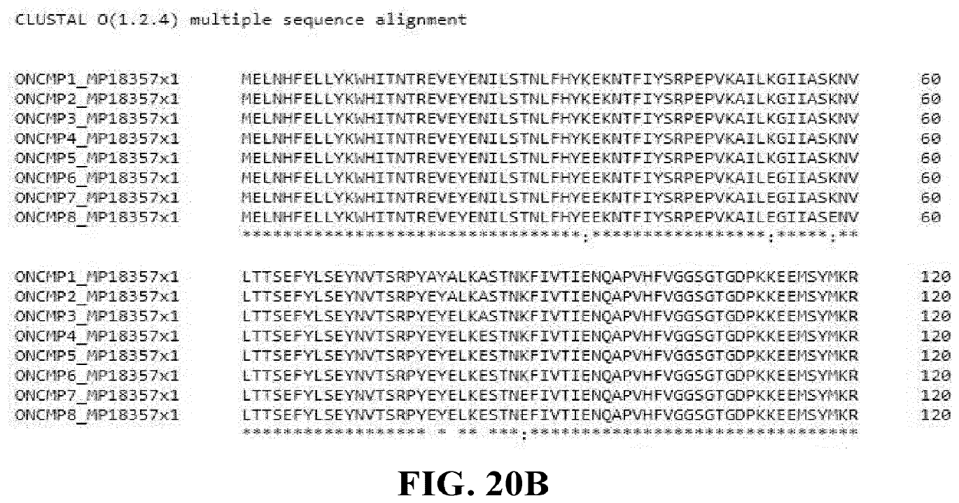

In some embodiments, the fusion protein comprises an onconase polypeptide comprising the amino acid sequence of any one of SEQ ID NOs: 2-10 or 15-22, optionally with one, two, three, four, five or all six C-terminal histidine residues deleted as shown in FIG. 20A. In some embodiments, the fusion protein comprises an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 2. In some embodiments, the fusion protein comprises an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 3. In some embodiments, the fusion protein comprises an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 4. In some embodiments, the fusion protein comprises an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 5. In some embodiments, the fusion protein comprises an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 6. In some embodiments, the fusion protein comprises an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 7. In some embodiments, the fusion protein comprises an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 7. In some embodiments, the fusion protein comprises an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 8. In some embodiments, the fusion protein comprises an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 9. In some embodiments, the fusion protein comprises an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 10. In some embodiments, the fusion protein comprises an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 15. In some embodiments, the fusion protein comprises an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 16. In some embodiments, the fusion protein comprises an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 17. In some embodiments, the fusion protein comprises an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 18. In some embodiments, the fusion protein comprises an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 19. In some embodiments, the fusion protein comprises an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 20. In some embodiments, the fusion protein comprises an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 21. In some embodiments, the fusion protein comprises an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 22. In some embodiments, the fusion polypeptide comprises an onconase polypeptide comprising at least one amino acid sequence that has between 80% to 85%, 85% to 90%, 90% to 95%, or 95% to 100% sequence identity with any one of SEQ ID NOs: 2-10 or 15-22, optionally with one, two, three, four, five or all six C-terminal histidine residues deleted as shown in FIG. 20A.

In some embodiments, the fusion polypeptide comprises an onconase polypeptide with a modified N-terminus and/or C-terminus. In some embodiments, the fusion polypeptide comprises an onconase polypeptide one or more amino acid substitution in the N-terminus and/or C-terminus as compared to SEQ ID NO: 1. In some embodiments, the fusion polypeptide comprises an onconase polypeptide comprising 11 amino acid substitutions in the N-terminus and/or C-terminus as compared to SEQ ID NO: 1. In some embodiments, the fusion polypeptide comprises an onconase polypeptide 11 amino acid substitutions in the N-terminus as compared to SEQ ID NO: 1.

In some embodiments, the fusion polypeptide comprises a TAF12 polypeptide comprising the amino acid sequence of SEQ ID NO: 23 operably linked to two or more oligopeptides by a peptide bond. In some embodiments, the fusion polypeptide comprises a TAF12 polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to SEQ ID NO: 23. In some embodiments, the fusion polypeptide comprises a TAF12 polypeptide comprising an amino acid sequence that has between 80% to 85%, 85% to 90%, 90% to 95%, or 95% to 100% sequence identity to SEQ ID NO: 23. In some embodiments, the TAF12 polypeptide is a truncated TAF12 polypeptide.

In some embodiments, the fusion polypeptide comprises a variant TAF12 polypeptide operably linked to two or more oligopeptides by a peptide bond. The variant TAF12 may be a TAF12 with mutations that decreased susceptibility to protein cleavage, increase yield, promote aggregation, or promote solubility. The modifications may be one or more of amino acid substitutions, deletions, or insertions. Other amino acid substitutions, insertions or deletions may be introduced that increase stability, yield or aggregation of the TAF12 protein.

In some embodiments, the fusion polypeptide comprises a variant TAF12 comprising one or more amino acid substitutions as compared to the amino acid sequence of SEQ ID NO: 23. In some embodiments, the one or more amino acid substitutions are conservative amino acid substitutions. Conservative amino acid substitutions refer to the substitution of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. In some embodiments, the one or more amino acid substitutions are non-conservative amino acid substitutions. Non-conservative substitutions refer to substitutions between amino acids with different properties (e.g., a non-charged residue substituted by a charged residue). In some embodiments, the fusion protein comprises a TAF12 polypeptide comprising one or more insertions or deletions as compared to the amino acid sequence of SEQ ID NO: 23. The one or more insertions or deletions may be of any amino acid length.

In some embodiments, the fusion protein comprises a TAF12 polypeptide with one or more amino acid substitutions that decrease susceptibility to chemical cleavage as compared to a TAF12 polypeptide having the amino acid sequence of SEQ ID NO: 23. In some embodiments, the fusion protein comprises a TAF12 polypeptide with one or more deletions or insertions that reduce susceptibility to chemical cleavage as compared to a TAF12 polypeptide having the amino acid sequence of SEQ ID NO: 23.

In some embodiments, the fusion protein comprises a TAF12 polypeptide with one or more amino acid substitutions that increase yield or promote formation of inclusion bodies as compared to a TAF12 polypeptide having the amino acid sequence of SEQ ID NO: 23. In some embodiments, the one or more amino acid substitutions that increase yield or promote formation of inclusion bodies comprise one or more substitutions of cationic or polar amino acids for non-polar or neutral amino acids. In some embodiments, the TAF12 comprises one or more amino acid substitutions, insertions, or deletions that reduce solubility. In some embodiments, the one or more amino acid substitutions result in a variant TAF12 polypeptide having a decreased number of cationic or polar amino acids as compared to a TAF12 polypeptide having the amino acid sequence of SEQ ID NO: 23. In some embodiments, the one or more amino acid substitutions that increase yield or promote formation of inclusion bodies result in a variant TAF12 having a decreased number of Lys, Arg, Cys, His, Asp, Glu, and/or Tyr as compared to a TAF12 polypeptide having the amino acid sequence of SEQ ID NO: 23. In some embodiments, the fusion protein comprises a TAF12 polypeptide with one or more amino acid substitutions that result in a TAF12 protein having an altered pI as compared to a TAF12 polypeptide having the amino acid sequence of SEQ ID NO: 23. In some embodiments, the fusion protein comprises a TAF12 polypeptide with one or more deletions or insertions that increase yield or promote formation of inclusion bodies as compared to a TAF12 polypeptide having the amino acid sequence of SEQ ID NO: 23. In some embodiments, the one or more insertions or deletions that increase yield or promote formation of inclusion bodies result in a variant TAF12 having a decreased number of cationic or polar amino acids as compared to a TAF12 polypeptide having the amino acid sequence of SEQ ID NO: 23. In some embodiments, the one or more insertions or deletions that increase yield or promote formation of inclusion bodies result in a variant TAF12 having a decreased number of Lys, Arg, Cys, His, Asp, Glu, and/or Tyr as compared to a TAF12 polypeptide having the amino acid sequence of SEQ ID NO: 23. In some embodiments, the fusion protein comprises a TAF12 polypeptide with one or more deletions or insertions that increase yield or promote formation of inclusion bodies as compared to a TAF12 polypeptide having the amino acid sequence of SEQ ID NO: 23. In some embodiments, the fusion protein comprises a TAF12 polypeptide with one or more deletions or insertions that result in a TAF12 protein having an altered pI as compared to a TAF12 polypeptide having the amino acid sequence of SEQ ID NO: 23. In any of the above embodiments, the TAF12 polypeptide may comprise an amino acid sequence that has at least 80%, at least 85%, at least 90%, at least 91%, at least 92%, at least 93%, at least 94%, at least 95%, at least 96%, at least 97%, at least 98%, or at least 99% sequence identity to SEQ ID NO: 23. In some embodiments, the TAF12 polypeptide comprises an amino acid sequence that has between 80% to 85%, 85% to 90%, 90% to 95%, or 95% to 100% sequence identity to SEQ ID NO: 23.

In some embodiments, the fusion protein comprises a TAF12 polypeptide with one or more N-terminal amino acid substitutions compared to SEQ ID NO: 23. In some embodiments, the fusion protein comprises a TAF12 polypeptide with 1, 2, 3, 4, 5, 6, 8, 9, 10, 11, 12, 13, 14, 15, 20, 25, 30, or 35 N-terminal amino acid substitutions compared to SEQ ID NO: 23. In some embodiments, the fusion protein comprises a TAF12 polypeptide with 11 N-terminal amino acid substitutions compared to SEQ ID NO: 23. In some embodiments, the amino acid substitutions are within the first 50 N-terminal amino acids, within the first 40 N-terminal amino acids, within the first 30 N-terminal amino acids, within the first 20 N-terminal amino acids, or within the first 11 amino acids. In some embodiments, the amino acid substitutions are within the first 15 N-terminal amino acids, within the first 14 N-terminal amino acids, within the first 13 N-terminal amino acids, within the first 12 N-terminal amino acids, within the first 11 N-terminal amino acids, within the first 10 N-terminal amino acids, within the first 9 N-terminal amino acids, within the first 8 N-terminal amino acids, within the first 7 N-terminal amino acids, within the first 6 N-terminal amino acids, within the first 5 N-terminal amino acids, within the first 4 N-terminal amino acids, within the first 3 N-terminal amino acids, or within the first 2 N-terminal amino acids. In some embodiments, one or more N-terminal amino acid substitutions are conservative amino acid substitutions. In some embodiments, one or more N-terminal amino acid substitutions are non-conservative amino acid substitutions. In some embodiments, the fusion protein comprises a TAF12 polypeptide with one or more N-terminal insertions or deletions compared to SEQ ID NO: 23. In some embodiments, the fusion polypeptide comprises a TAF12 polypeptide comprises the amino acid sequence of SEQ ID NO: 23. In some embodiments, the fusion polypeptide comprises a TAF12 polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to SEQ ID NO: 23. In some embodiments, the fusion polypeptide comprises a TAF12 polypeptide comprising an amino acid sequence that has between 80% to 85%, 85% to 90%, 90% to 95%, or 95% to 100% sequence identity to SEQ ID NO: 23.

### Peptide Release

In some embodiments, the method for producing oligopeptides comprises releasing the two or more oligopeptides from the insoluble carrier polypeptide (e.g., an onconase polypeptide or a TAF12 polypeptide) by sequence specific cleavage of a peptide bond. In some embodiments, the sequence specific cleavage of the peptide bond is performed by acidic cleavage. In some embodiments, the acidic cleavage is performed by incubation with acetic acid or formic acid. In some embodiments, the incubation is at elevated temperature, such as above room temperature.

In some embodiments, the oligopeptides are operably linked by the peptide bond and are released from each other when the oligopeptides are released from the onconase. In some embodiments, the oligopeptides are operably linked by a different peptide bond and are released from each other by sequence-specific chemical cleavage of the different peptide bond after the oligopeptides are released from the onconase.

In some embodiments, the step of releasing the two or more oligopeptides from the insoluble carrier polypeptide (e.g., an onconase polypeptide or a TAF12 polypeptide) is performed in the presence of a chaotropic agent. A chaotropic agent is a molecule that can disrupt the hydrogen bonding network between water molecules. It is theorized that addition of a chaotropic agent may reduce the order of the structure of a protein, thus promoting unfolding of the protein. Examples of chaotropic agents include, without limitation, guanidine hydrochloride (guanidinium hydrochloride, GdnHCl), sodium thiocyanate, n-butanol, ethanol, lithium perchlorate, lithium acetate, magnesium chloride, phenol, 2-propanol, thiourea, sodium dodecyl sulfate, and urea. The chaotropic agent may also be a detergent which disrupts the noncovalent intermolecular bonding within a protein, permitting the amino acid chain to assume a substantially random conformation.

In some embodiments, the step of releasing the two or more oligopeptides from the insoluble carrier polypeptide (e.g., an onconase polypeptide or a TAF12 polypeptide) is performed in the absence of a chaotropic agent. The addition of a chaotropic agent might not always be desired in protein purification protocols. For instance, addition of a chaotropic agent may require additional steps or conditions to be added to a protocol. These additional steps may increase the amount of time, reagents and cost of a purification protocol, or may negatively affect protein yield or purity.

The fusion polypeptide may be expressed in any suitable cell. The fusion polypeptide may be expressed in a bacterial, yeast, plant, or animal cell. In some embodiments, the fusion polypeptide is expressed in a unicellular organism. In some embodiments, the fusion polypeptide is expressed in a microbial cell. Any cell or cell culture known to produce inclusion bodies is suitable for expression of the fusion peptide. For example, the fusion polypeptide may be expressed in strains of Escherichia coli and Vibrio natriegens. Suitable yeast cells, include, without limitation, strains of Pichia pastoris. The bacterial strain may be a strain that lacks Lon and ompT protease function (e.g. a BL21 E. coli strain). In some embodiments, the fusion polypeptide is expressed in a bacteria in a bacterial cell culture. In some embodiments, the bacteria is an E. coli strain.

Expression of the polypeptide may be driven by any suitable promoter known in the art. The promoter may be a constitutive promoter or an inducible promoter. Inducible promoters refer to those regulated promoters that can be turned on by an external stimulus (e.g., a chemical, nutritional stress, or heat). For example, the lac promoter can be induced through use of lactose or IPTG (isopropylthio-β-D-galactoside). Alternatively, the promoter may be a constitutive promoter, which directs expression of the fusion polypeptide in a particular organisms, cell or tissue. The strength of the promoter, whether inducible or constitutive, may vary. For instance, a promoter may be a high expression promoter, or strong promoter, which results in overexpression of a given gene. In some embodiments, the fusion polypeptide is expressed with a strong promoter. In some embodiments, the promoter drives the expression of the fusion protein in aggregates.

In some embodiments, the method further comprises measuring the yield of released oligopeptides. Protein yield may be measured by any known methods for measuring peptide yield. For instance, peptide yield may be measured in terms of peptide mass obtained from a liter of cell culture, or g/L. In some embodiments, the yield of oligopeptide is at least 10 mg/L of bacterial culture, at least 20 mg/L of bacterial culture, at least 30 mg/L of bacterial culture, at least 40 mg/L of bacterial culture, at least 50 mg/L of bacterial culture. In some embodiments, the yield of oligopeptide is between 10 mg/L to 200 mg/L of bacterial culture, between 20 mg/L to 150 mg/L of bacterial culture, between 30 mg/L to 120 mg/L of bacterial culture, between 40 mg/L to 120 mg/L of bacterial culture, between 50 mg/L to 120 g/L of bacterial culture, between 50 g/L to 100 g/L of bacterial culture, between 60 g/L to 90 g/L of bacterial culture, between 70 g/L to 110 g/L of bacterial culture, or between 40 g/L to 80 g/L of bacterial culture. In some embodiments, the yield of the expressed fusion polypeptide is measured after harvesting cells at stationary phase.

In some embodiments, the method may comprise expressing the fusion polypeptide in a cell culture in a bioreactor. Use of a bioreactor may lead to higher yields of released oligopeptides. Large scale cell growth and fusion polypeptide expression may utilize a wide range of simple or complex carbohydrates, organic acids or alcohols, and saturated hydrocarbons such as methane. Fusion protein encoding gene expression may be regulated, repressed or depressed by specific growth conditions, which may include the form and amount of nitrogen, phosphorous, sulfur, oxygen, carbon or any trace micronutrient including small inorganic ions. In addition, the regulation may be achieved by the presence or absence of specific regulatory molecules that are added to the culture and are not typically considered nutrient or energy sources.

II. Methods of Releasing Oligopeptides from Insoluble Carrier Polypeptide, which Form Inclusion Bodies

Some aspects of the present disclosure provide for methods of releasing an oligopeptide fused to an insoluble carrier polypeptide, which forms inclusion bodies in a cell.

Inclusion bodies are intracellular amorphous deposit comprising aggregated protein found in the cytoplasm or periplasm of a cell. Oligopeptides of interest that are typically soluble with the cell and/or cell lysates can be linked to an insoluble carrier polypeptide, which forms inclusion bodies to facilitate formation of insoluble aggregates that include the oligopeptides. Trapped into inclusion bodies, the oligopeptides are inaccessible to cellular proteases and may accumulate in large quantities. Examples of insoluble carrier polypeptides, which forms inclusion bodies include, without limitation, a trpΔLE polypeptide (Derynck R et al. (1984)), a ketosteroid isomerase polypeptide (Kuliopulos et al. (1994)), a β-galactosidase polypeptide (Schellenberger V et al. (1993)), a PagP polypeptide (Hwang P M et al. (2012)), EDDIE (Achmuller C et al. (2007)), the cleavable self-aggregating tag INTEIN-ELK16 (Zhao Q et al. (2016)), GFIL8 (Wang X. et al. (2015)), a PaP3.30 polypeptide (Rao X C et al. (2004)), TAF12-HFD (Vidovic V et al. (2012)), and the F4 fragment of PurF (Lee J H et al. (2000)). Other exemplary insoluble carrier polypeptides, which forms inclusion bodies include modified versions of a E. coli maltose-binding protein (Betton and Hofnug, J. Biol. Chem. 271:8046-8052 (1996)), an E. coli RNAse II polypeptide (Coburn and Mackie, J. Biol. Chem. 271:1048-1053 (1996)), an E. coli alkaline phosphatase polypeptide (Derman and Beckwith, J Bacteriol. 177:3764-3770 (1995); Georgiou et al., Appl. Env. Microbial. 52:1157-1161 (1986)), an E. coli phospholipase A polypeptide (Dekker et al., Eur. J. Biochem. 232:214-219 (1995)), an E. coli β-lactamase polypeptide (Rinas and Bailey, Appl. Env. Microbiol. 59:561-566 (1993); Georgiou et al., Appl. Env. Microbiol. 52:1157-1161 (1986)), a Salmonella typhimurium MalK protein (Schneider et al., Prot. Exp. Purif. 6:10-14 (1995)), a Clostridium thermocellum endoglucanase D polypeptide (Tokatlidis et al., FEBS Lett. 282:205-208 (1991)), a Bacillus thuringiensis subsp. aizawai IPL7 insecticidal proteins (Oeda et al., J. Bacteriol. 171:3568-3571 (1989), a human procathepsin B polypeptide (Kuhelj et al., Eur. J. Biochem. 229:533-539 (1995)), a porcine interferon-γ polypeptide (Vandenbroeck et al., Eur. J. Biochem. 215:481-486 (1993)), a T5 DNA polymerase polypeptide (Chatterjee et al., Gene 97:13-19 (1991)), and E. coli thioredoxin (Hoog et al., BioSci. Rep. 4:917-923 (1984)). In some embodiments, the insoluble carrier polypeptide is an onconase polypeptide or a TAF12 polypeptide.

In some embodiments, the methods of releasing an oligopeptide fused to an insoluble carrier polypeptide, which forms inclusion bodies in a cell comprises expressing a fusion polypeptide comprising the oligopeptide operably linked to the insoluble carrier polypeptide, which forms inclusion bodies in a cell; and releasing the two or more oligopeptides from the insoluble carrier polypeptide (e.g., an onconase polypeptide or a TAF12 polypeptide) by sequence-specific chemical cleavage of the peptide bond.

In some embodiments, the method comprises purifying the inclusion bodies. The inclusion bodies may be purified by lysing the cells to extract the inclusion bodies. The cells may be lysed using any number of means, including mechanical and/or chemical lysis. Any mechanical methods of purifying inclusion bodies from a cell lysate known in the art may be used, including centrifugation, filtration, sonication, French press, shearing, and any combinations thereof).

The inclusion bodies may also be isolated by solubilization of the aggregates using, for example, micelles or reverse micelles as described in Vinogradov, et al. (2003) Anal Biochem. 15; 320(2):234-8. In some embodiments, purifying the inclusion bodies comprises centrifuging the inclusion bodies. In some embodiments, centrifuging the inclusion bodies results in separation of the inclusion bodies from the cell lysate. In some embodiments, the inclusion bodies are solubilized in a solution comprising a chemical cleavage agent after purification. In some embodiments, the inclusion bodies are solubilized in a chaotropic agent-free solution after purification. In some embodiments, the method comprises purifying inclusion bodies formed by a fusion protein comprising an onconase fused to two or more oligopeptide. In some embodiments, the fusion protein is expressed in bacteria or yeast. In some embodiments, the fusion protein comprises a cleavable Asp-Pro bond. In some embodiments, the oligopeptides are 4-50 or 5-30 amino acids long. In some embodiments, the onconase is a variant onconase.

In some embodiments, the method of purifying inclusion bodies comprises expressing the fusion protein in a cell, purifying the inclusion bodies, and incubating the inclusion bodies with acetic acid, further comprises incubating the inclusion bodies with acetic acid. In some embodiments, the inclusion bodies are incubated with acetic acid at a high temperature. In some embodiments, the temperature is greater than 50° C., greater than 60° C., greater than 70° C., greater than 80° C., or greater than 90° C. In some embodiments, the temperature is less than 100° C., less than 95° C., less than 90° C., or less than 85° C. In some embodiments, the temperature is between 50° C. to 60° C., between 60° C. to 70° C., between 70° C. to 80° C., between 80° C. to 90° C., between 50-100, or between 90° C. to 100° C. In some embodiments, the method comprises expressing a fusion polypeptide comprising an insoluble carrier polypeptide (e.g., an onconase polypeptide or a TAF12 polypeptide) operably linked to two or more polypeptides by a peptide bond. In some embodiments, the peptide bond is capable of sequence-specific chemical cleavage. In some embodiments, the two or more oligopeptides are operably linked to the insoluble carrier polypeptide (e.g., an onconase polypeptide or a TAF12 polypeptide) by a peptide bond comprising an Asp-Pro bond and the sequence-specific chemical cleavage is with formic acid or acetic acid.

In some embodiments, the inclusion bodies are incubated with acetic acid at a temperature of greater than 50° C. for at least one hour. The acetic acid concentration may be at least two percent by weight, at least three percent by weight, at least four percent by weight, or at least five percent by weight. In some embodiments, the acetic acid concentration is less than fifty percent by weight, less than forty-five percent by weight, less than forty percent by weight, less than thirty-five percent by weight, or less than thirty percent by weight. In some embodiments, the acetic acid concentration is less than fifty percent by weight, less than forty-five percent by weight, less than forty percent by weight, less than thirty-five percent by weight, or less than thirty percent by weight. In some embodiments, the acetic acid concentration is less than fifty percent by weight, less than forty-five percent by weight, less than forty percent by weight, less than thirty-five percent by weight, or less than thirty percent by weight. In some embodiments, the acetic acid concentration is between ten to twenty percent by weight, between twenty to thirty percent by weight, between thirty to forty percent by weight, or between forty to fifty percent by weight. In some embodiments, the inclusion bodies are incubated for 1-24 hours in acetic acid. In some embodiments, the inclusion bodies are incubated in acetic acid between 2 to 4 hours, between 4 to 6 hours, between 6 to 8 hours, between 8 to 10 hours, between 10 to 14 hours, between 14 to 18 hours, between 18 to 20 hours, or between 20 to 24 hours. In some embodiments, the inclusion bodies are incubated in acetic acid for about 2 hours, about 4 hours, about 6 hours, about 8 hours, about 10 hours, about 12 hours, about 14 hours, about 16 hours, about 18 hours, about 20 hours or about 24 hours. In some embodiments, the inclusion bodies are incubated in acetic acid for up to 20 hours. In some embodiments, the method comprises expressing a fusion polypeptide comprising an insoluble carrier polypeptide (e.g., an onconase polypeptide or a TAF12 polypeptide) operably linked to two or more polypeptides by a peptide bond. In some embodiments, the peptide bond is capable of sequence-specific chemical cleavage. In some embodiments, the two or more oligopeptides are operably linked to the insoluble carrier polypeptide (e.g., an onconase polypeptide or a TAF12 polypeptide) by a peptide bond comprising an Asp-Pro bond and the sequence-specific chemical cleavage is with formic acid or acetic acid.

In some embodiments, the method of purifying inclusion bodies comprises expressing the fusion protein in a cell, purifying the inclusion bodies, and incubating the inclusion bodies with acetic acid, further comprises releasing the two or more oligopeptides from the insoluble carrier polypeptide, which forms inclusion bodies in a cell by sequence-specific chemical cleavage. In some embodiments, releasing the two or more oligopeptides from the insoluble carrier polypeptide, which forms inclusion bodies in a cell is performed using an acid. In some embodiments, the acid is acetic acid or formic acid. In some embodiments, releasing the two or more oligopeptides from the insoluble carrier polypeptide (e.g., an onconase polypeptide or a TAF12 polypeptide) by sequence-specific acetic acid cleavage of the peptide bond. In some embodiments, cleavage of the fusion polypeptide by acetic acid results in the release of the insoluble carrier polypeptide, which forms inclusion bodies in a cell from the two or more polypeptides and/or the release of the two or more oligopeptides from each other. In some embodiments, releasing the two or more oligopeptides from the insoluble carrier polypeptide, which forms inclusion bodies in a cell occurs after solubilization of the inclusion bodies. In some embodiments, releasing the two or more oligopeptides from the insoluble carrier polypeptide, which forms inclusion bodies in a cell occurs during incubation of the inclusion bodies in acetic acid. In some embodiments, the release of the peptides is not complete. For example, in some embodiments, dipeptides or tripeptides remain after incubation with acetic acid or formic acid.

In some embodiments, the method further comprises separating the released oligopeptides from the insoluble carrier polypeptide, which forms inclusion bodies in a cell. After the release step, the oligopeptides may be separated and/or isolated from the insoluble carrier polypeptide, which forms inclusion bodies based on a differential solubility of the components. Parameters such as pH, salt concentration, and temperature may be adjusted to facilitate separation of the oligopeptides from the insoluble carrier polypeptide, which forms inclusion bodies. The released oligopeptide may be further purified using any known purification techniques in the art such, for example, as ion exchange, gel purification techniques, differential centrifugation, and column chromatography.

In some embodiments, the method of purifying inclusion bodies comprises expressing the fusion protein in a cell, purifying the inclusion bodies, and incubating the inclusion bodies with acetic acid, wherein the fusion polypeptide comprises expressing the fusion polypeptide in conditions that promote the formation of inclusion bodies. In some embodiments, the step of expressing the fusion polypeptide is performed at neutral pH. In some embodiments, expressing the fusion polypeptide at neutral pH results in the formation of inclusion bodies. In some embodiments, the step of expressing the fusion polypeptide is performed at high temperatures. In some embodiments, expressing the fusion polypeptide at high temperature results in the formation of inclusion bodies. In some embodiments, expressing the fusion polypeptide at high levels results in the formation of inclusion bodies. In some embodiments, the step of expressing the fusion polypeptide is performed using a cell that is prone to formation of inclusion bodies. In some embodiments, expressing the fusion polypeptide in a cell that is prone to formation of inclusion bodies results in the formation of inclusion bodies.

In some embodiments, provided herein is a method of purifying inclusion bodies comprising a fusion protein, wherein the fusion protein comprises an insoluble carrier polypeptide (e.g., an onconase polypeptide or a TAF12 polypeptide) operably linked to two or more oligopeptides. In some embodiments, the method comprises expressing a fusion polypeptide comprising an insoluble carrier polypeptide (e.g., an onconase polypeptide or a TAF12 polypeptide) operably linked to two or more polypeptides by a peptide bond. In some embodiments, the peptide bond is capable of sequence-specific chemical cleavage. In some embodiments, the two or more oligopeptides are operably linked to the insoluble carrier polypeptide (e.g., an onconase polypeptide or a TAF12 polypeptide) by a peptide bond comprising an Asp-Pro bond and the sequence-specific chemical cleavage is with formic acid or acetic acid.

In some embodiments, the method of purifying inclusion bodies comprising a fusion protein comprises expressing a fusion polypeptide comprising two or more, three or more oligopeptides, four or more oligopeptides, five or more oligopeptides, six or more polypeptides, eight or more polypeptides, ten or more polypeptides, fifteen or more polypeptides, or twenty or more oligopeptides operably linked to the insoluble carrier polypeptide (e.g., an onconase polypeptide or a TAF12 polypeptide). In some embodiments, the method comprises expressing a fusion polypeptide comprising an insoluble carrier polypeptide (e.g., an onconase polypeptide or a TAF12 polypeptide) and two or more oligopeptides operably linked to the N-terminus or the C-terminus of the onconase protein. In some embodiments, the method comprises expressing a fusion polypeptide comprising an insoluble carrier polypeptide (e.g., an onconase polypeptide or a TAF12 polypeptide) and two or more oligopeptides are linked to the C-terminus of the onconase. In some embodiments, two or more oligopeptides linked to the C-terminus of the onconase have the same amino acid sequence. In some embodiments, two or more oligopeptides linked to the C-terminus of the onconase have a different amino acid sequence. In some embodiments, the method comprises expressing a fusion polypeptide comprising an insoluble carrier polypeptide (e.g., an onconase polypeptide or a TAF12 polypeptide) and two or more oligopeptides are linked to the N-terminus of the onconase. In some embodiments, the two or more oligopeptides linked to the N-terminus of the onconase have the same amino acid sequence. In some embodiments, the two or more oligopeptides linked to the N-terminus of the onconase have a different amino acid sequence. In some embodiments, the inclusion bodies are solubilized in the absence of a chaotropic agent. In some embodiments, the onconase is an onconase mutant. In some embodiments the onconase mutant comprises one or more amino acid substitutions, insertions, or deletions in the 15 N-terminal amino acids.

In some embodiments, the method of purifying inclusion bodies comprises expressing the fusion protein in a cell, purifying the inclusion bodies, and incubating the inclusion bodies with acetic acid at, for example, a temperature of at least 50° C. for at least an hour, wherein the fusion polypeptide comprises two or more oligopeptides that are less than 25 amino acids in length. In some embodiments, the two or more oligopeptides are at least four, at least five, at least six, at least seven, at least eight, at least nine, at least ten, at least fifteen, at least twenty, or at least twenty-five amino acids long. In some embodiments, the two or more oligopeptides are up to fifty amino acids in length. In some embodiments, the two or more oligopeptides are less than fifty, less than forty-five, less than forty, less than thirty-five, less than thirty, less than twenty-five, or less than twenty amino acids long. In some embodiments, the two or more oligopeptides are between four and fifty, between six and forty amino acids long, between six and thirty amino acids long, or between eight and twenty-five amino acids long. In some embodiments, the inclusion bodies are solubilized in the absence of a chaotropic agent. In some embodiments, the inclusion bodies are incubated with acetic acid at a temperature of greater than 50° C. for at least one hour.

In some embodiments, the method of purifying inclusion bodies comprises expressing the fusion protein in a cell, purifying the inclusion bodies, and incubating the inclusion bodies with acetic acid, wherein the fusion polypeptide comprises an insoluble carrier polypeptide (e.g., an onconase polypeptide or a TAF12 polypeptide) linked to two or more micropeptides (miPEPs). In some embodiments, the two or more oligopeptides comprise a miPEP sequence that regulates a miRNA. In some embodiments, the two or more oligopeptides comprise a miPEP sequence that regulates two or more miRNA families. In some embodiments, the two or more oligopeptides comprise a miPEP sequence that regulates a plant miRNA. In some embodiments, the two or more oligopeptides comprise a miPEP sequence that regulates a plant miRNA selected from the group consisting of the miR156, miR159/319, miR160, miR162, miR164, miR166, miR167, miR168, miR169, miR171, miR172, miR390, miR393, miR394, miR295, miR396, miR397, miR398, miR408, miR403, miR437, miR444, and miR445 families. In some embodiments, the one or more oligopeptides comprise a miPEP sequence that has microbe inhibiting activity. In some embodiments, the onconase is a variant onconase. In some embodiments, the two or more oligopeptides are 4-50 or 5-30 amino acids long. In some embodiments, the fusion polypeptide is expressed in a yeast or bacterial cell. In some embodiments, the inclusion bodies are solubilized in the absence of a chaotropic agent. In some embodiments, the inclusion bodies are incubated with acetic acid at a temperature of greater than 50° C. for at least one hour.

In some embodiments, the method of purifying inclusion bodies comprises expressing the fusion protein in a cell, purifying the inclusion bodies, and incubating the inclusion bodies with acetic acid, wherein the fusion polypeptide further comprises a linker sequence between the two or more oligopeptides and/or between the insoluble carrier polypeptide (e.g., an onconase polypeptide or a TAF12 polypeptide) and the oligopeptides. In some embodiments, the linker sequence is capable of being cleaved by sequence-specific chemical cleavage. In some embodiments, the linker sequence separates the two or more oligopeptides and/or a linker sequence separating the insoluble carrier polypeptide (e.g., an onconase polypeptide or a TAF12 polypeptide) from the two or more oligopeptides. In some embodiments, the linker sequence separating the two or more oligopeptides has the same or different sequence than the linker sequence separating the insoluble carrier polypeptide (e.g., an onconase polypeptide or a TAF12 polypeptide) from the two or more oligopeptides. In some embodiments, the method comprises expressing a fusion polypeptide comprising a linker sequence comprising the amino acid sequence of SEQ ID NO: 11 separating the two or more oligopeptides and/or separating the insoluble carrier polypeptide (e.g., an onconase polypeptide or a TAF12 polypeptide) from the two or more oligopeptides. In some embodiments, the method comprises expressing a fusion polypeptide comprising a linker sequence comprising an Asp-Pro linker separating the two or more oligopeptides and/or separating the insoluble carrier polypeptide (e.g., an onconase polypeptide or a TAF12 polypeptide) from the two or more oligopeptides. In some embodiments, the inclusion bodies are solubilized in the absence of a chaotropic agent. In some embodiments, the inclusion bodies are incubated with acetic acid at a temperature of greater than 50° C. for at least one hour.

In some embodiments, the method of purifying inclusion bodies comprises expressing the fusion protein in a cell, purifying the inclusion bodies, and incubating the inclusion bodies with acetic acid, wherein the fusion polypeptide comprises an onconase polypeptide comprising the amino acid sequence of SEQ ID NO: 1 operably linked to two or more oligopeptides by a peptide bond. In some embodiments, the method comprises expressing a fusion polypeptide comprising an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to SEQ ID NO: 1. In some embodiments, the method comprises expressing a fusion polypeptide comprising an onconase polypeptide comprising an amino acid sequence that has between 80% to 85%, 85% to 90%, 90% to 95%, or 95% to 100% sequence identity to SEQ ID NO: 1. In some embodiments, the method comprises expressing a fusion polypeptide comprising a variant onconase comprising one or more amino acid substitutions as compared to the amino acid sequence of SEQ ID NO: 1. In some embodiments, the method comprises expressing a fusion polypeptide comprising an onconase polypeptide comprising one or more insertions or deletions as compared to the amino acid sequence of SEQ ID NO: 1. The one or more insertions or deletions may be of any amino acid length. In some embodiments, the inclusion bodies are solubilized in the absence of a chaotropic agent. In some embodiments, the inclusion bodies are incubated with acetic acid at a temperature of greater than 50° C. for at least one hour.

In some embodiments, the method of purifying inclusion bodies comprises expressing the fusion protein in a cell, purifying the inclusion bodies, and incubating the inclusion bodies with acetic acid, wherein the fusion polypeptide comprises an onconase polypeptide with one or more amino acid substitutions that decrease susceptibility to chemical cleavage as compared to an onconase polypeptide having the amino acid sequence of SEQ ID NO: 1. In some embodiments, the method comprises expressing a fusion polypeptide comprising an onconase polypeptide with one or more deletions or insertions that reduce susceptibility to chemical cleavage as compared to an onconase polypeptide having the amino acid sequence of SEQ ID NO: 1. In some embodiments, the inclusion bodies are solubilized in the absence of a chaotropic agent. In some embodiments, the inclusion bodies are incubated with acetic acid at a temperature of greater than 50° C. for at least one hour.

In some embodiments, the method of purifying inclusion bodies comprises expressing the fusion protein in a cell, purifying the inclusion bodies, and incubating the inclusion bodies with acetic acid, wherein the fusion polypeptide comprises an onconase polypeptide with a modified N-terminus and/or C-terminus. In some embodiments, the method comprises expressing a fusion polypeptide comprising an onconase polypeptide one or more amino acid substitution in the N-terminus and/or C-terminus as compared to SEQ ID NO: 1. In some embodiments, the method comprises expressing a fusion polypeptide comprising an onconase polypeptide comprising 11 amino acid substitutions in the N-terminus and/or C-terminus as compared to SEQ ID NO: 1. In some embodiments, the method comprises expressing a fusion polypeptide comprising an onconase polypeptide 11 amino acid substitutions in the N-terminus as compared to SEQ ID NO: 1. In some embodiments, the inclusion bodies are solubilized in the absence of a chaotropic agent. In some embodiments, the inclusion bodies are incubated with acetic acid at a temperature of greater than 50° C. for at least one hour.

In some embodiments, the method of purifying inclusion bodies comprises expressing the fusion protein in a cell, purifying the inclusion bodies, and incubating the inclusion bodies with acetic acid, wherein the fusion polypeptide comprises a TAF12 polypeptide comprising the amino acid sequence of SEQ ID NO: 23 operably linked to two or more oligopeptides by a peptide bond. In some embodiments, the method comprises expressing a fusion polypeptide comprising a TAF12 polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to SEQ ID NO: 23. In some embodiments, the method comprises expressing a fusion polypeptide comprising a TAF12 polypeptide comprising an amino acid sequence that has between 80% to 85%, 85% to 90%, 90% to 95%, or 95% to 100% sequence identity to SEQ ID NO: 23. In some embodiments, the method comprises expressing a fusion polypeptide comprising a variant TAF12 comprising one or more amino acid substitutions as compared to the amino acid sequence of SEQ ID NO: 23. In some embodiments, the method comprises expressing a fusion polypeptide comprising a TAF12 polypeptide comprising one or more insertions or deletions as compared to the amino acid sequence of SEQ ID NO: 23. The one or more insertions or deletions may be of any amino acid length. In some embodiments, the inclusion bodies are solubilized in the absence of a chaotropic agent. In some embodiments, the inclusion bodies are incubated with acetic acid at a temperature of greater than 50° C. for at least one hour.

In some embodiments, the method of purifying inclusion bodies comprises expressing the fusion protein in a cell, purifying the inclusion bodies, and incubating the inclusion bodies with acetic acid, wherein the fusion polypeptide comprises a TAF12 polypeptide with one or more amino acid substitutions that decrease susceptibility to chemical cleavage as compared to a TAF12 polypeptide having the amino acid sequence of SEQ ID NO: 23. In some embodiments, the method comprises expressing a fusion polypeptide comprising a TAF12 polypeptide with one or more deletions or insertions that reduce susceptibility to chemical cleavage as compared to a TAF12 polypeptide having the amino acid sequence of SEQ ID NO: 23. In some embodiments, the inclusion bodies are solubilized in the absence of a chaotropic agent. In some embodiments, the inclusion bodies are incubated with acetic acid at a temperature of greater than 50° C. for at least one hour.

In some embodiments, the method of purifying inclusion bodies comprises expressing the fusion protein in a cell, purifying the inclusion bodies, and incubating the inclusion bodies with acetic acid, wherein the fusion polypeptide comprises a TAF12 polypeptide with a modified N-terminus and/or C-terminus. In some embodiments, the method comprises expressing a fusion polypeptide comprising a TAF12 polypeptide one or more amino acid substitution in the N-terminus and/or C-terminus as compared to SEQ ID NO: 23. In some embodiments, the method comprises expressing a fusion polypeptide comprising a TAF12 polypeptide comprising 11 amino acid substitutions in the N-terminus and/or C-terminus as compared to SEQ ID NO: 23. In some embodiments, the method comprises expressing a fusion polypeptide comprising a TAF12 polypeptide 11 amino acid substitutions in the N-terminus as compared to SEQ ID NO: 23. In some embodiments, the inclusion bodies are solubilized in the absence of a chaotropic agent. In some embodiments, the inclusion bodies are incubated with acetic acid at a temperature of greater than 50° C. for at least one hour.

### III. Methods of Purifying Inclusion Bodies Comprising a Fusion Protein.

Some aspects of the present disclosure provide for methods of purifying inclusion bodies comprising a fusion protein. In some embodiments, the method comprises expressing a fusion protein comprising an oligopeptide operably linked to a protein that forms inclusion bodies (such as an onconase), lysing a cell to form a lysate, centrifuging the cell lysate to form a pellet, washing the pellet in a surfactant buffer, washing the pellet in a salt buffer, and washing the pellet in water. In some embodiments, the method comprises purifying inclusion bodies formed by a fusion protein comprising an onconase fused to two or more oligopeptides. In some embodiments, the fusion protein is expressed in bacteria or yeast. In some embodiments, the fusion protein comprises a cleavable Asp-Pro bond. In some embodiments, the oligopeptides are 4-50 or 5-30 amino acids long. In some embodiments, the onconase is an onconase mutant.

In some embodiments, the method of purifying inclusion bodies comprising a fusion polypeptide comprises lysing the cell to form a lysate. Cell lysis may be performed through any methods known to those skilled in the art. In some embodiments, cell lysis is performed by enzymatic lysis. Methods for enzymatic lysis include, but are not limited to, enzymatic lysis using lytic enzymes such as lysozyme, lysostaphin, mutanolysin, or any other enzyme that disrupts a cell wall. In some embodiments, cell lysis is performed by mechanical methods. Methods of mechanical lysis include without limitation, physical shearing, such as with glass beads, sonication, French press, ultrasound, incubation with a hypotonic solution, or high pressure. In some embodiments, the cell lysate is a yeast or bacterial cell lysate. In some embodiments, the fusion protein comprises an onconase fused to two or more oligopeptides. In some embodiments, the oligopeptides are 4-50 or 5-30 amino acids long. In some embodiments, the onconase is a variant onconase.

In some embodiments, the method of purifying inclusion bodies comprising a fusion protein comprises centrifuging the cell lysate to form a pellet. In some embodiments, centrifugation is performed at a speed of about 500×g, about, 1,000×g, about 5,000×g, about 10,000×g, about 15,000×g, about 20,000×g, or about 25,000×g. In some embodiments, centrifugation is performed at a speed of between 500×g to 1,000×g, 1,000×g to 5,000×g, 5,000×g to 10,000×g, 10,000×g to 15,000×g, 15,000×g to 20,000×g, or 20,000×g to 25,000×g. In some embodiments, centrifugation is performed at a speed of about 10,000×g. In some embodiments, centrifugation is performed at a temperature of about 4° to 10° C. In some embodiments, centrifugation is performed at room temperature. In some embodiments, centrifugation is performed for about 5 to 60 minutes. In some embodiments, centrifugation is performed for about 5 to 10 minutes, about 10 to 15 minutes, about 15 to 30 minutes, about 30 to 45 minutes, or about 45 to 60 minutes. In some embodiments, centrifugation is performed for about 5 minutes, about 10 minutes, about 15 minutes, about 20 minutes, about 30 minutes, about 45 minutes or about 60 minutes. In some embodiments, centrifugation is performed for longer than 60 minutes. In some embodiments, centrifugation of the cell lysate results in a pellet comprising inclusion bodies. In some embodiments, centrifugation of the cell lysate results in a pellet that is free of cell debris. In some embodiments, the cell lysate is a yeast or bacterial cell lysate. In some embodiments, the fusion protein comprises an onconase fused to two or more oligopeptides. In some embodiments, the oligopeptides are 4-50 or 5-30 amino acids long. In some embodiments, the onconase is a variant onconase.

In some embodiments, the method of purifying inclusion bodies comprising a fusion protein comprises washing the pellet in a surfactant buffer. A surfactant is a surface active compound which reduces the surface tension of water and includes, nonionic (including, but not limited to, t-octylphenoxypolyethoxy-ethanol and polyoxyethylene sorbitan), anionic (e.g., sodium dodecyl sulfate) and cationic (e.g., cetylpyridinium chloride) and amphoteric agents. Suitable surfactants include, but are not limited to deoxycholate, sodium octyl sulfate, sodium tetradecyl sulfate, polyoxyethylene ethers, sodium cholate, octylthioglucopyranoside, n-octylglucopyranoside, alkyltrimethylammonium bromides, alkyltrimethyl ammonium chlorides, sodium bis(2-ethylhexyl)sulfosuccinate. In some embodiments, the surfactant buffer comprises a nonionic surfactant. In some embodiments, the surfactant is TritonX-100. In some embodiments, the pellet is washed at least once, at least twice, or at least three times with the surfactant. In some embodiments, the pellet is washed more than three times with the surfactant. In some embodiments, the fusion protein comprises an insoluble carrier polypeptide (e.g., an onconase polypeptide or a TAF12 polypeptide) operably linked to two or more oligopeptides by an Asp-Pro bond. In some embodiments, the oligopeptides are 4-50 or 5-30 amino acids long. In some embodiments, the onconase is a variant onconase.

In some embodiments, the purified inclusion bodies incubated at about 95° C. following washing with the surfactant. In some embodiments the incubation denatures the proteins in the inclusion bodies. In some embodiments, the incubation is carried out for at least 1, at least 2, at least 3 at least 5, at least 10 or at least 20 minutes. In some embodiments, the incubation is carried out for 1-20 minutes. In some embodiments, the incubation is at between about 90° C. to about 100° C.

In some embodiments, the method of purifying inclusion bodies comprising a fusion protein comprises washing the pellet in a buffer with salt. In some embodiments, the buffer has a high salt level. Such buffers are advantageous for disaggregating and/or refolding mixtures and to maintain a desired pH value or pH range. Inorganic salt buffers (e.g., phosphate, carbonate, sodium, among others) and organic salt buffers (e.g., citrate, Tris, MOPS, MES, HEPES, among others) are well known to the art. In some embodiments, the salt buffer comprises NaCl. In some embodiments, the salt buffer comprises at least 0.5M NaCl. In some embodiments, the salt buffer is at least 0.6M NaCl, at least 0.7M NaCl, at least 0.75M NaCl, or at least 1 M NaCl. In some embodiments, the salt buffer is between 0.1M to 0.6 M NaCl, between 0.6M to 0.7M NaCl, between 0.7M to 0.8M NaCl, between 0.8M to 0.9M NaCl, or between 0.9M to 1M NaCl. In some embodiments, the pellet is washed with the buffer at least once, at least twice, or at least three times. In some embodiments, the pellet is washed with the buffer more than three times. In some embodiments, the fusion protein comprises an onconase fused to two or more oligopeptides. In some embodiments, the oligopeptides are 4-50 or 5-30 amino acids long. In some embodiments, the onconase is a variant onconase. In some embodiments, the purified inclusion bodies are incubated at 95° C. following washing with the buffer.

In some embodiments, the method of purifying inclusion bodies comprising a fusion protein further comprises washing the pellet in water. Washing a pellet with water may remove or diminish any remaining impurities, as well as any remaining salt or surfactants molecules in the solution. In some embodiments, the pellet is washed in water at least once, at least twice, or at least three times. In some embodiments, the pellet is washed in water more than three times. In some embodiments, washing the pellet with water leads to purified inclusion bodies. In some embodiments, the fusion protein comprises an onconase fused to two or more oligopeptides. In some embodiments, the oligopeptides are 4-50 or 5-30 amino acids long. In some embodiments, the onconase is a variant onconase. In some embodiments, the purified inclusion bodies are incubated at 95° C. following washing with water.

In some embodiments, the method of purifying inclusion bodies comprising a fusion protein comprises further comprises solubilizing the inclusion bodies. Solubilization may be performed in the presence or absence of a chaotropic agent (e.g., urea or guanidinium hydrochloride) or on the absence of a chaotropic agent. In some embodiments, method of purifying inclusion bodies comprising a fusion protein further comprises solubilizing the inclusion bodies in a chaotropic agent-free solution. In some embodiments, method of purifying inclusion bodies comprising a fusion protein further comprises solubilizing the inclusion bodies in a solution comprising a chemical cleavage reagent. In some embodiments, method of purifying inclusion bodies comprising a fusion protein further comprises solubilizing the inclusion bodies in a solution comprising acidic acid or formic acid. In some embodiments, the inclusion bodies are solubilized in acetic acid. In some embodiments, the inclusion bodies comprise a fusion protein which comprises an onconase fused to two or more oligopeptides. In some embodiments, the oligopeptides are 4-50 or 5-30 amino acids long. In some embodiments, the onconase is a variant onconase.

In some embodiments, the method of purifying inclusion bodies comprising a fusion protein comprises purifying the inclusion bodies. Suitable techniques for purification include, but are not limited to, ammonium sulfate or ethanol precipitation, acid extraction, electrophoresis, immunoadsorption, anion or cation exchange chromatography, phosphocellulose chromatography, hydrophobic interaction chromatography, affinity chromatography, immunoaffinity chromatography, size exclusion chromatography, liquid chromatography (LC), high performance LC (HPLC), fast performance LC (FPLC), hydroxyapatite chromatography and lectin chromatography. In some embodiments, the method of purifying inclusion bodies comprising a fusion protein further comprises purifying the inclusion bodies by chromatography. In some embodiments, the method of purifying inclusion bodies comprising a fusion protein does not comprise purifying the inclusion bodies by chromatography.

In some embodiments, the method of purifying inclusion bodies comprising a fusion protein comprises expressing a fusion protein comprising an oligopeptide operably linked to a protein that forms inclusion bodies in a cell. The protein that forms inclusion bodies in a cell may be any insoluble carrier polypeptide, which forms inclusion bodies in a cell. In some embodiments, the protein that forms inclusion bodies in a cell is an onconase. In some embodiments, the inclusion bodies comprise a fusion polypeptide comprising an insoluble carrier polypeptide (e.g., an onconase polypeptide or a TAF12 polypeptide) operably linked to one or more polypeptides by a peptide bond. In some embodiments, the peptide bond is capable of sequence-specific chemical cleavage. In some embodiments, the oligopeptide is 4-50 or 5-30 amino acids long. In some embodiments, the fusion protein comprises a cleavable Asp-Pro bond.

In some embodiments, the method of purifying inclusion bodies comprising a fusion protein comprises purifying inclusion bodies comprising a fusion polypeptide comprising an insoluble carrier polypeptide (e.g., an onconase polypeptide or a TAF12 polypeptide) operably linked to two or more oligopeptide. In some embodiments, the fusion protein comprises two or more, three or more oligopeptides, four or more oligopeptides, five or more oligopeptides, six or more polypeptides, eight or more polypeptides, ten or more polypeptides, fifteen or more polypeptides, or twenty or more oligopeptides operably linked to the insoluble carrier polypeptide (e.g., an onconase polypeptide or a TAF12 polypeptide). In some embodiments, the inclusion bodies comprise a fusion polypeptide comprising an insoluble carrier polypeptide (e.g., an onconase polypeptide or a TAF12 polypeptide) and one or more oligopeptides operably linked to the N-terminus or the C-terminus of the onconase protein. In some embodiments, the inclusion bodies comprise a fusion polypeptide comprising an insoluble carrier polypeptide (e.g., an onconase polypeptide or a TAF12 polypeptide) and one or more oligopeptides are linked to the C-terminus of the onconase. In some embodiments, one or more oligopeptides linked to the C-terminus of the onconase have the same amino acid sequence. In some embodiments, one or more oligopeptides linked to the C-terminus of the onconase have a different amino acid sequence. In some embodiments, the inclusion bodies comprise a fusion polypeptide comprising an insoluble carrier polypeptide (e.g., an onconase polypeptide or a TAF12 polypeptide) and one or more oligopeptides are linked to the N-terminus of the onconase. In some embodiments, one or more oligopeptides linked to the N-terminus of the onconase have the same amino acid sequence. In some embodiments, one or more oligopeptides linked to the N-terminus of the onconase have a different amino acid sequence. In some embodiments, the onconase is a variant onconase.

In some embodiments, the method of purifying inclusion bodies comprising a fusion protein comprises purifying inclusion bodies comprising a fusion polypeptide comprising an oligopeptide operably linked to a protein that forms inclusion bodies in a cell, wherein the operable linkage is a chemically cleavable amino acid sequence. In some embodiments, the two or more oligopeptides are 4-50 or 5-30 amino acids long. In some embodiments, the inclusion bodies comprise a fusion polypeptide comprising one or more oligopeptides that are less than 25 amino acids in length. In some embodiments, the one or more oligopeptides are at least four, at least five, at least six, at least seven, at least eight, at least nine, at least ten, at least fifteen, at least twenty, or at least twenty-five amino acids long. In some embodiments, the one or more oligopeptides are up to fifty amino acids in length. In some embodiments, the one or more oligopeptides are less than fifty, less than forty-five, less than forty, less than thirty-five, less than thirty, less than twenty-five, or less than twenty amino acids long. In some embodiments, the one or more oligopeptides are between four and fifty, between six and forty amino acids long, between six and thirty amino acids long, or between eight and twenty-five amino acids long. In some embodiments, the fusion polypeptide comprises a cleavable Asp-Pro bond. In some embodiments, the method comprises lysing the cell to form a lysate, centrifuging the cell lysate to form a pellet, and washing the pellet in a surfactant buffer comprising a nonionic surfactant.

In some embodiments, the method of purifying inclusion bodies comprising a fusion protein comprises purifying inclusion bodies comprising a fusion polypeptide comprising an insoluble carrier polypeptide (e.g., an onconase polypeptide or a TAF12 polypeptide) linked to one or more micropeptides (miPEPs). In some embodiments, the one or more oligopeptides comprise a miPEP sequence that regulates one or more miRNA families. In some embodiments, the one or more oligopeptides comprise a miPEP sequence that regulates a plant miRNA family. In some embodiments, the method comprises lysing the cell to form a lysate, centrifuging the cell lysate to form a pellet, and washing the pellet in a surfactant buffer comprising a nonionic surfactant.

In some embodiments, the method of purifying inclusion bodies comprising a fusion protein comprises purifying inclusion bodies comprising a fusion polypeptide further comprising a linker sequence. In some embodiments, the linker sequence is capable of being cleaved by sequence-specific chemical cleavage. In some embodiments, the linker sequence separates the one or more oligopeptides and/or a linker sequence separating the insoluble carrier polypeptide (e.g., an onconase polypeptide or a TAF12 polypeptide) from the one or more oligopeptides. In some embodiments, the linker sequence separating the one or more oligopeptides has the same or different sequence than the linker sequence separating the insoluble carrier polypeptide (e.g., an onconase polypeptide or a TAF12 polypeptide) from the one or more oligopeptides. In some embodiments, the inclusion bodies comprise a fusion polypeptide comprising a linker sequence comprising the amino acid sequence of SEQ ID NO: 11 separating the one or more oligopeptides and/or separating the insoluble carrier polypeptide (e.g., an onconase polypeptide or a TAF12 polypeptide) from the one or more oligopeptides. In some embodiments, the inclusion bodies comprise a fusion polypeptide comprising a linker sequence comprising an Asp-Pro linker separating the one or more oligopeptides and/or separating the insoluble carrier polypeptide (e.g., an onconase polypeptide or a TAF12 polypeptide) from the one or more oligopeptides. In some embodiments, the method comprises lysing the cell to form a lysate, centrifuging the cell lysate to form a pellet, and washing the pellet in a surfactant buffer comprising a nonionic surfactant.

In some embodiments, the method of purifying inclusion bodies comprising a fusion protein comprises purifying inclusion bodies comprising a fusion polypeptide comprising an onconase polypeptide comprising the amino acid sequence of SEQ ID NO: 1 operably linked to one or more oligopeptides by a peptide bond. In some embodiments, the inclusion bodies comprise a fusion polypeptide comprising an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to SEQ ID NO: 1. In some embodiments, the one or more oligopeptides are 4-50 or 5-30 amino acids long. In some embodiments, the fusion protein comprises a cleavable Asp-Pro bond.

In some embodiments, the methods comprise purifying inclusion bodies comprising a fusion polypeptide comprising a variant onconase comprising one or more amino acid substitutions as compared to the amino acid sequence of SEQ ID NO: 1 operably linked to one or more oligopeptides. In some embodiments, the inclusion bodies comprise a fusion polypeptide comprising an onconase polypeptide comprising one or more insertions or deletions as compared to the amino acid sequence of SEQ ID NO: 1. In some embodiments, the method of purifying inclusion bodies comprising a fusion protein comprises purifying inclusion bodies comprising a fusion polypeptide comprising an onconase polypeptide with one or more amino acid substitutions that decrease susceptibility to chemical cleavage as compared to an onconase polypeptide having the amino acid sequence of SEQ ID NO: 1. In some embodiments, the inclusion bodies comprise a fusion polypeptide comprising an onconase polypeptide with one or more deletions or insertions that reduce susceptibility to chemical cleavage as compared to an onconase polypeptide having the amino acid sequence of SEQ ID NO: 1. In some embodiments, the inclusion bodies comprise a fusion polypeptide comprising an onconase polypeptide with one or more amino acid substitutions that increase yield or promote formation of inclusion bodies as compared to an onconase polypeptide having the amino acid sequence of SEQ ID NO: 1. In some embodiments, the inclusion bodies comprise a fusion polypeptide comprising an onconase polypeptide with one or more deletions or insertions that increase yield or promote formation of inclusion bodies as compared to an onconase polypeptide having the amino acid sequence of SEQ ID NO: 1. In some embodiments, the method comprises lysing the cell to form a lysate, centrifuging the cell lysate to form a pellet, and washing the pellet in a surfactant buffer comprising a nonionic surfactant.

In some embodiments, the inclusion bodies comprise a fusion polypeptide comprising an onconase polypeptide comprising the amino acid sequence of any one of SEQ ID NOs: 2-10 and 15-22, optionally with one, two, three, four, five or all six C-terminal histidine residues deleted as shown in FIG. 20A. In some embodiments, the inclusion bodies comprise a fusion polypeptide comprising an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to any of SEQ ID NOs: 2-10 and 15-22, optionally with one, two, three, four, five or all six C-terminal histidine residues deleted as shown in FIG. 20A. In some embodiments, the fusion polypeptide comprises an onconase polypeptide comprising an amino acid sequence that has between 80% to 85%, 85% to 90%, 90% to 95%, or 95% to 100% sequence identity with any one of SEQ ID NOs: 2-10 and 15-22, optionally with one, two, three, four, five or all six C-terminal histidine residues deleted as shown in FIG. 20A. In some embodiments, the one or more oligopeptides are 4-50 or 5-30 amino acids long. In some embodiments, the fusion protein comprises a cleavable Asp-Pro bond. In some embodiments, the method comprises lysing the cell to form a lysate, centrifuging the cell lysate to form a pellet, and washing the pellet in a surfactant buffer comprising a nonionic surfactant.

In some embodiments, the inclusion bodies comprise a fusion polypeptide comprising an onconase polypeptide with a modified N-terminus and/or C-terminus operably linked to one or more oligopeptides. In some embodiments, the inclusion bodies comprise a fusion polypeptide comprising an onconase polypeptide one or more amino acid substitution in the N-terminus and/or C-terminus as compared to SEQ ID NO: 1. In some embodiments, the inclusion bodies comprise a fusion polypeptide comprising an onconase polypeptide comprising 11 amino acid substitutions in the N-terminus and/or C-terminus as compared to SEQ ID NO: 1. In some embodiments, the inclusion bodies comprise a fusion polypeptide comprising an onconase polypeptide 11 amino acid substitutions in the N-terminus as compared to SEQ ID NO: 1. In some embodiments, the one or more oligopeptides are 4-50 or 5-30 amino acids long. In some embodiments, the fusion protein comprises a cleavable Asp-Pro bond. In some embodiments, the method comprises lysing the cell to form a lysate, centrifuging the cell lysate to form a pellet, and washing the pellet in a surfactant buffer comprising a nonionic surfactant.

In some embodiments, the method of purifying inclusion bodies comprising a fusion protein comprises purifying inclusion bodies comprising a fusion polypeptide comprising a TAF12 polypeptide comprising the amino acid sequence of SEQ ID NO: 23 operably linked to one or more oligopeptides by a peptide bond. In some embodiments, the inclusion bodies comprise a fusion polypeptide comprising a TAF12 polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to SEQ ID NO: 23. In some embodiments, the one or more oligopeptides are 4-50 or 5-30 amino acids long. In some embodiments, the fusion protein comprises a cleavable Asp-Pro bond.

In some embodiments, the methods comprise purifying inclusion bodies comprising a fusion polypeptide comprising a variant TAF12 comprising one or more amino acid substitutions as compared to the amino acid sequence of SEQ ID NO: 23 operably linked to one or more oligopeptides. In some embodiments, the inclusion bodies comprise a fusion polypeptide comprising a TAF12 polypeptide comprising one or more insertions or deletions as compared to the amino acid sequence of SEQ ID NO: 23. In some embodiments, the method of purifying inclusion bodies comprising a fusion protein comprises purifying inclusion bodies comprising a fusion polypeptide comprising a TAF12 polypeptide with one or more amino acid substitutions that decrease susceptibility to chemical cleavage as compared to a TAF12 polypeptide having the amino acid sequence of SEQ ID NO: 23. In some embodiments, the inclusion bodies comprise a fusion polypeptide comprising a TAF12 polypeptide with one or more deletions or insertions that reduce susceptibility to chemical cleavage as compared to a TAF12 polypeptide having the amino acid sequence of SEQ ID NO: 23. In some embodiments, the inclusion bodies comprise a fusion polypeptide comprising a TAF12 polypeptide with one or more amino acid substitutions that increase yield or promote formation of inclusion bodies as compared to a TAF12 polypeptide having the amino acid sequence of SEQ ID NO: 23. In some embodiments, the inclusion bodies comprise a fusion polypeptide comprising a TAF12 polypeptide with one or more deletions or insertions that increase yield or promote formation of inclusion bodies as compared to a TAF12 polypeptide having the amino acid sequence of SEQ ID NO: 23. In some embodiments, the method comprises lysing the cell to form a lysate, centrifuging the cell lysate to form a pellet, and washing the pellet in a surfactant buffer comprising a nonionic surfactant.

In some embodiments, the inclusion bodies comprise a fusion polypeptide comprising a TAF12 polypeptide with a modified N-terminus and/or C-terminus operably linked to one or more oligopeptides. In some embodiments, the inclusion bodies comprise a fusion polypeptide comprising a TAF12 polypeptide one or more amino acid substitution in the N-terminus and/or C-terminus as compared to SEQ ID NO: 23. In some embodiments, the inclusion bodies comprise a fusion polypeptide comprising a TAF12 polypeptide comprising 11 amino acid substitutions in the N-terminus and/or C-terminus as compared to SEQ ID NO: 23. In some embodiments, the inclusion bodies comprise a fusion polypeptide comprising a TAF12 polypeptide 11 amino acid substitutions in the N-terminus as compared to SEQ ID NO: 23. In some embodiments, the one or more oligopeptides are 4-50 or 5-30 amino acids long. In some embodiments, the fusion protein comprises a cleavable Asp-Pro bond. In some embodiments, the method comprises lysing the cell to form a lysate, centrifuging the cell lysate to form a pellet, and washing the pellet in a surfactant buffer comprising a nonionic surfactant.

### IV. Onconase Polypeptides

Also provided herein are onconase polypeptides for use as insoluble carrier peptides.

Onconase, also known as ranpirnase, is an RNAse that was first identified in Rana pipiens. Onconase is a protein of about 104 amino acids in length, stabilized by four disulfide bridges, and undergoes pH-dependent denaturation. Without being limited by theory, acidic conditions promote denaturation of onconase, and the denatured form onconase is highly soluble, and efficient renaturation can only be achieved by a method based on reversible blocking of cysteine residues. At neutral conditions (around pH 7), onconase is prone to aggregation, and high yield production of onconase can lead to formation of insoluble aggregates, such as inclusion bodies, with low to undetectable soluble levels of the protein. Once aggregated, the onconase may be removed from a solution by selective precipitation. Moreover, onconase lacks any aspartate-proline and asparagine-glycine sequences, which makes it resistant to common chemical cleavage strategies.

In some embodiments, the onconase polypeptide comprises the amino acid sequence of SEQ ID NO: 1. In some embodiments, the onconase polypeptide comprises an amino acid sequence that has at least 80%, at least 85%, at least 90%, at least 91%, at least 92%, at least 93%, at least 94%, at least 95%, at least 96%, at least 97%, at least 98%, or at least 99% sequence identity to SEQ ID NO: 1. In some embodiments, the onconase polypeptide comprises an amino acid sequence that has between 80% to 85%, 85% to 90%, 90% to 95%, or 95% to 100% sequence identity to SEQ ID NO: 1. In some embodiments, the onconase polypeptide is a truncated onconase polypeptide. In some embodiments, the truncation is at the N-terminus.

In some embodiments, the onconase polypeptide has one or more amino acid substitutions that decrease its pI. In some embodiments, the onconase polypeptide has a pI of about 8.0, about 7.5, about 7.0, or about 6.5. In some embodiments, the onconase polypeptide has a neutral pI. In some embodiments, the onconase polypeptide has a pI of less than 7.0.

In some embodiments, the onconase polypeptide comprises one or more amino acid substitutions compared to the amino acid sequence of SEQ ID NO: 1. In some embodiments, the one or more amino acid substitutions are conservative amino acid substitutions. In some embodiments, the one or more amino acid substitutions are non-conservative amino acid substitutions. In some embodiments, the fusion protein comprises an onconase polypeptide comprising one or more insertions or deletions as compared to the amino acid sequence of SEQ ID NO: 1. In some embodiments, the onconase polypeptide is inactive.

In some embodiments, the onconase polypeptide comprises one or more amino acid substitutions that decrease susceptibility to chemical cleavage as compared to an onconase polypeptide having the amino acid sequence of SEQ ID NO: 1. In some embodiments, the onconase polypeptide comprises one or more deletions or insertions that reduce susceptibility to chemical cleavage as compared to an onconase polypeptide having the amino acid sequence of SEQ ID NO: 1. In some embodiments, the onconase polypeptide has 1, 2, 3, 4, 5, 6, 7, 8, 9, 10, 11, 12, 13, 14, 15, 16, 17, 18, 19, 20 or more amino acid substitutions, insertions, or deletions compared to an onconase polypeptide having the amino acid sequence of SEQ ID NO: 1.

In some embodiments, the onconase polypeptide comprises one or more amino acid substitutions that increase yield or promote formation of inclusion bodies as compared to an onconase polypeptide having the amino acid sequence of SEQ ID NO: 1. In some embodiments, the onconase polypeptide comprises one or more amino acid substitutions that increase yield or promote formation of inclusion bodies comprise one or more substitutions of cationic or polar amino acids for non-polar or neutral amino acids. In some embodiments, the one or more amino acid substitutions result in a variant onconase polypeptide that has a decreased number of cationic or polar amino acids as compared to an onconase polypeptide having the amino acid sequence of SEQ ID NO: 1. In some embodiments, the onconase polypeptide comprises one or more deletions or insertions that increase yield or promote formation of inclusion bodies as compared to an onconase polypeptide having the amino acid sequence of SEQ ID NO: 1. In some embodiments, the one or more insertions or deletions that increase yield or promote formation of inclusion bodies result in a variant onconase having a decreased number of cationic or polar amino acids as compared to an onconase polypeptide having the amino acid sequence of SEQ ID NO: 1.

In some embodiments, the onconase polypeptide comprises mutation of one or more charged or polar amino acids to a non-polar amino acid. In some embodiments, the onconase polypeptide comprises one or more alanine substitutions. In some embodiments, the onconase polypeptide comprises one or more lysine to alanine substitutions. In some embodiments, the onconase polypeptide comprises 2, 3, 4, 5, 6, 7, 8, 9, or 10 alanine substitutions. In some embodiments, the onconase polypeptide does not contain lysine residues. In some embodiments, the onconase polypeptide does not contain arginine residues. In some embodiments, the onconase polypeptide does not contain lysine or arginine residues.

In some embodiments, the onconase polypeptide comprises a modified N-terminus and/or C-terminus. In some embodiments, the onconase polypeptide comprises one or more amino acid substitution in the N-terminus and/or C-terminus as compared to SEQ ID NO: 1. In some embodiments, the onconase polypeptide comprises 11 amino acid substitutions in the N-terminus and/or C-terminus as compared to SEQ ID NO: 1. In some embodiments, the onconase polypeptide comprises 11 amino acid substitutions in the N-terminus as compared to SEQ ID NO: 1.

In some embodiments, the onconase polypeptide comprises the amino acid sequence of any one of SEQ ID NOs: 2-10, optionally with one, two, three, four, five or all six C-terminal histidine residues deleted as shown in FIG. 20A. In some embodiments, the onconase polypeptide comprises an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 2. In some embodiments, the onconase polypeptide comprises an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 3. In some embodiments, the onconase polypeptide comprises an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 4. In some embodiments, the onconase polypeptide comprises an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 5. In some embodiments, the onconase polypeptide comprises an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 6. In some embodiments the onconase polypeptide comprises an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 7. In some embodiments, the onconase polypeptide comprises an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 7. In some embodiments the onconase polypeptide comprises an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 8. In some embodiments, the onconase polypeptide comprises an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 9. In some embodiments, the onconase polypeptide comprises an amino acid sequence that has at least 80%, at least 85%, at least 90%, at least 91%, at least 92%, at least 93%, at least 94%, at least 95%, at least 96%, at least 97%, at least 98%, or at least 99% sequence identity to the amino acid sequence set forth in SEQ ID NO: 10. In each case, the above may optionally have one, two, three, four, five or all six C-terminal histidine residues deleted as shown in FIG. 20A.

In some embodiments, the onconase polypeptide comprises an amino acid sequence tag. In some embodiments, the amino acid sequence tag is a purification amino acid sequence tag. In some embodiments, the amino acid sequence tag is a detection amino acid sequence tag. In some embodiments, the tag is less than 20 amino acids in length, less than 10 amino acids in length, or less than 5 amino acids in length. In some embodiments, the tag is 5-20 amino acids in length. In some embodiments, the tag is a hexahistidine or FLAG tag. In some embodiments, the tag is a biotin tag.

In some embodiments, the onconase polypeptide has increased expression levels as compared to an onconase polypeptide having the amino acid sequence of SEQ ID NO: 1. Expression levels can be measured by any standard techniques known in the art. For instance, the amount of protein (in grams) can be compared to the amount in grams of total cell protein in a given sample, with the measurement determined as a level of recombinant protein per liter. The level or amount can also be measured as compared to a known standard, such as a BSA control. Other techniques to measure protein levels include without limitation, light absorption analysis of a purified protein samples, antibody-based detection (e.g., western blot, FACS, immunofluorescence), activity measurements, and microscopy analysis (e.g., phase contrast, Nomarski interference, electron or fluorescence microscopy). The level or activity can also be compared to a known standard (such as a known amount of purified, active protein) for more accurate quantification.

Also provided herein are compositions comprising an onconase mutant polypeptide described herein.

### V. Fusion Polypeptides

Provided herein are also fusion polypeptides comprising a polypeptide that can form inclusion bodies and one or more oligopeptides. Any of the polypeptides that form inclusion bodies disclosed herein may be used. In some embodiments, the polypeptide that forms inclusion bodies is an insoluble carrier polypeptide (e.g., an onconase polypeptide or a TAF12 polypeptide). In some embodiments, the carrier is a variant carrier. In some embodiments, the fusion polypeptide comprises an insoluble carrier polypeptide (e.g., an onconase polypeptide or a TAF12 polypeptide) operably linked to one or more oligopeptides. In some embodiments, the one or more oligopeptides are operably linked to the insoluble carrier polypeptide (e.g., an onconase polypeptide or a TAF12 polypeptide) by a cleavable bond. In some embodiments, the one or more oligopeptides are 4-50 or 5-30 amino acids long. In some embodiments, the fusion protein comprises a cleavable Asp-Pro bond.

In some embodiments, the fusion polypeptide comprises two or more, three or more oligopeptides, four or more oligopeptides, five or more oligopeptides, six or more polypeptides, eight or more polypeptides, ten or more polypeptides, fifteen or more polypeptides, or twenty or more oligopeptides operably linked to the insoluble carrier polypeptide (e.g., an onconase polypeptide or a TAF12 polypeptide). In some embodiments, the fusion polypeptide comprises an insoluble carrier polypeptide (e.g., an onconase polypeptide or a TAF12 polypeptide) and one or more oligopeptides are operably linked to the N-terminus and/or the C-terminus of the onconase protein. In some embodiments, the one or more oligopeptides are each 4-50 or 5-30 amino acids long.

In some embodiments, the fusion polypeptide comprises an insoluble carrier polypeptide (e.g., an onconase polypeptide or a TAF12 polypeptide) and one or more oligopeptides operably linked to the N-terminus or the C-terminus of the onconase protein. In some embodiments, the fusion polypeptide comprises three or more oligopeptides, four or more oligopeptides, five or more oligopeptides, six or more polypeptides, eight or more polypeptides, ten or more polypeptides, fifteen or more polypeptides, or twenty or more oligopeptides operably linked to N-terminus and/or the C-terminus of the insoluble carrier polypeptide (e.g., an onconase polypeptide or a TAF12 polypeptide). In some embodiments, the fusion polypeptide comprises an insoluble carrier polypeptide (e.g., an onconase polypeptide or a TAF12 polypeptide) and one or more oligopeptides are linked to the C-terminus of the onconase. In some embodiments, one or more oligopeptides linked to the C-terminus of the onconase have the same amino acid sequence. In some embodiments, one or more oligopeptides linked to the C-terminus of the onconase have a different amino acid sequence. In some embodiments, the fusion polypeptide comprises an insoluble carrier polypeptide (e.g., an onconase polypeptide or a TAF12 polypeptide) and one or more oligopeptides are linked to the N-terminus of the onconase. In some embodiments, one or more oligopeptides linked to the N-terminus of the onconase have the same amino acid sequence. In some embodiments, one or more oligopeptides linked to the N-terminus of the onconase have a different amino acid sequence.

In some embodiments, the fusion polypeptide comprises an insoluble carrier polypeptide (e.g., an onconase polypeptide or a TAF12 polypeptide) operably linked to the one or more oligopeptides by a cleavable peptide bond. The cleavable bond may be cleaved by enzymatic or chemical agents. Cleavage of the peptide bond may result in separation of the insoluble carrier polypeptide (e.g., an onconase polypeptide or a TAF12 polypeptide) from the oligopeptides and/or of the oligopeptides from each other. In some embodiments, the peptide bond can be cleaved by enzymatic cleavage. In some embodiments, the peptide bond is capable of sequence-specific chemical cleavage. In some embodiments, the peptide bond includes (i) a methionine and the sequence-specific chemical cleavage is with cyanogen bromide, (ii) a tryptophan and the sequence-specific chemical cleavage is with BNPS-skatole, (iii) an aspartic acid-proline (Asp-Pro) bond and the sequence-specific chemical cleavage is with formic acid, (iv) an asparagine-glycine bond and the sequence-specific chemical cleavage is with hydroxylamine, or (v) a cysteine and the sequence-specific chemical cleavage is with NTCB. In some embodiments, the one or more oligopeptides are operably linked to the insoluble carrier polypeptide (e.g., an onconase polypeptide or a TAF12 polypeptide) by a peptide bond comprising an Asp-Pro bond and the sequence-specific chemical cleavage is with formic acid or acetic acid.

In some embodiments, the fusion polypeptide comprises an insoluble carrier polypeptide (e.g., an onconase polypeptide or a TAF12 polypeptide) operably linked to one or more oligopeptides that are 4-50 or 5-30 amino acids long. In some embodiments, the one or more oligopeptides are less than 25 amino acids in length. In some embodiments, the one or more oligopeptides are at least four, at least five, at least six, at least seven, at least eight, at least nine, at least ten, at least fifteen, at least twenty, or at least twenty-five amino acids long. In some embodiments, the one or more oligopeptides are up to fifty amino acids in length. In some embodiments, the one or more oligopeptides are less than fifty, less than forty-five, less than forty, less than thirty-five, less than thirty, less than twenty-five, or less than twenty amino acids long. In some embodiments, the one or more oligopeptides are between four and fifty, between six and forty amino acids long, between six and thirty amino acids long, or between eight and twenty-five amino acids long. In some embodiments, the one or more oligopeptides are operably linked to each other by an Asp-Pro bond. In some embodiments, the operable linkage between the insoluble carrier polypeptide (e.g., an onconase polypeptide or a TAF12 polypeptide) and the one or more oligopeptides comprises an Asp-Pro bond. In some embodiments, the insoluble carrier polypeptide (e.g., an onconase polypeptide or a TAF12 polypeptide) is a variant onconase.

In some embodiments, fusion polypeptide comprises an insoluble carrier polypeptide (e.g., an onconase polypeptide or a TAF12 polypeptide) operably linked to one or more oligopeptides comprise the amino acid sequence of any one of SEQ ID NOs: 12-14 and 31-2401. In some embodiments, the one or more oligopeptides comprise an amino acid sequence that has at least 80%, at least 85%, at least 90%, at least 91%, at least 92%, at least 93%, at least 94%, at least 95%, at least 96%, at least 97%, at least 98%, or at least 99% sequence identity with any one of SEQ ID NOs: 12-14 and 31-2401. In some embodiments, the one or more oligopeptides comprise an amino acid sequence that has between 80% to 85%, 85% to 90%, 90% to 95%, or 95% to 100% sequence identity with any one of SEQ ID NOs: 12-14 and 31-2401. In some embodiments, the one or more oligopeptides are 4-50 or 5-30 amino acids in length. In some embodiments, the onconase is a variant onconase. In some embodiments, the operable linkage comprises an Asp-Pro bond.

In some embodiments, the fusion polypeptide comprises an insoluble carrier polypeptide (e.g., an onconase polypeptide or a TAF12 polypeptide) linked to one or more micropeptides (miPEPs). In some embodiments, the one or more oligopeptides comprise a miPEP sequence that regulates a miRNA. In some embodiments, the one or more oligopeptides comprise a miPEP sequence that regulates a plant miRNA, a fungal miRNA, or a metazoan miRNA. In some embodiments, the one or more oligopeptides comprise a miPEP sequence that regulates a plant miRNA. In some embodiments, the one or more oligopeptides comprise a miPEP sequence that regulates a plant miRNA selected from the group consisting of the miR156, miR159/319, miR160, miR162, miR164, miR166, miR167, miR168, miR169, miR171, miR172, miR390, miR393, miR394, miR295, miR396, miR397, miR398, miR408, miR403, miR437, miR444, and miR445 families. In some embodiments, the one or more oligopeptides comprise a miPEP sequence that has microbe inhibiting activity. In some embodiments, the one or more oligopeptides are 4-50 or 5-30 amino acids in length. In some embodiments, the onconase is a variant onconase. In some embodiments, the operable linkage comprises an Asp-Pro bond.

In some embodiments, the fusion polypeptide further comprises a linker sequence between the one or more oligopeptides and/or between the insoluble carrier polypeptide (e.g., an onconase polypeptide or a TAF12 polypeptide) and the oligopeptides. The linker sequence may function as a spacer peptide to separate the one or more oligopeptides and/or the insoluble carrier polypeptide (e.g., an onconase polypeptide or a TAF12 polypeptide) and oligopeptides. In some embodiments, the linker sequence provides the cleavable peptide bond. For instance, the linker sequence may provide the cleavable peptide bond by incorporation an amino acid sequence that directs the sequence-specific cleavage of the fusion polypeptide. In some embodiments, the linker sequence is capable of being cleaved by sequence-specific chemical cleavage. In some embodiments, the fusion polypeptide comprises a linker sequence separating the one or more oligopeptides and/or a linker sequence separating the insoluble carrier polypeptide (e.g., an onconase polypeptide or a TAF12 polypeptide) from the one or more oligopeptides. In some embodiments, the linker sequence separating the one or more oligopeptides has the same or different sequence than the linker sequence separating the insoluble carrier polypeptide (e.g., an onconase polypeptide or a TAF12 polypeptide) from the one or more oligopeptides. In some embodiments, the fusion polypeptide comprises a linker sequence comprising the amino acid sequence of SEQ ID NO: 11 separating the one or more oligopeptides and/or separating the insoluble carrier polypeptide (e.g., an onconase polypeptide or a TAF12 polypeptide) from the one or more oligopeptides. In some embodiments, the fusion polypeptide comprises a linker sequence comprising an Asp-Pro linker separating the one or more oligopeptides and/or separating the insoluble carrier polypeptide (e.g., an onconase polypeptide or a TAF12 polypeptide) from the one or more oligopeptides. In some embodiments, sequence-specific chemical cleavage of the linker sequence results in the release of the insoluble carrier polypeptide (e.g., an onconase polypeptide or a TAF12 polypeptide) from the one or more oligopeptides and/or of the one or more oligopeptides from each other.

In some embodiments, the fusion polypeptide further comprises one or more amino acid sequence tags. In some embodiments, the amino acid sequence tag does not affect the solubility of the fusion polypeptide. In some embodiments, the amino acid sequence tag has a neutral pI. In some embodiments, the one or more amino acid sequence tags are purification amino acid sequence tags. Such tag peptides may include, but are not limited to, glutathione-S-transferase (GST), polyhistidine, maltose binding protein (MBP), avidin, biotin, streptavidin, histidine (His) tags (e.g. His-6X tag), V5 tags, FLAG tags, influenza hemagglutinin (HA) tags, Myc tags, VSV-G tags, thioredoxin (Trx) tags, and ligands for cellular receptor (e.g., insulin receptor ligands). In some embodiments, the one or more amino acid sequence tags sequence is a detection or reporter amino acid sequence tag. Examples of reporter amino acid sequence tag include without limitation horseradish peroxidase (HRP), chloramphenicol acetyltransferase (CAT) beta-galactosidase, beta-glucuronidase, luciferase, and fluorescent proteins (e.g., GFP, CFP, YFP, BFP, etc.). In some embodiments, the one or more amino acid sequence tags are operably linked to the N-terminus and/or C-terminus of the insoluble carrier polypeptide (e.g., an onconase polypeptide or a TAF12 polypeptide) or the one or more oligopeptides. In some embodiments, the one or more amino acid sequence tags are operably linked to the C-terminus of the insoluble carrier polypeptide (e.g., an onconase polypeptide or a TAF12 polypeptide) and the N-terminus of the one or more oligopeptides. In some embodiments, the one or more amino acid sequence tags are operably linked to the C-terminus of the insoluble carrier polypeptide (e.g., an onconase polypeptide or a TAF12 polypeptide) and the N-terminus of the one or more oligopeptides. In some embodiments, the one or more amino acid sequence tags are operably linked to the C-terminus of one oligopeptide and the N-terminus of another oligopeptide.

In some embodiments, the fusion polypeptide comprises an onconase polypeptide comprising the amino acid sequence of SEQ ID NO: 1 operably linked to one or more oligopeptides by a peptide bond. In some embodiments, the fusion polypeptide comprises an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to SEQ ID NO: 1. In some embodiments, the onconase polypeptide an amino acid sequence that has between 80% to 85%, 85% to 90%, 90% to 95%, or 95% to 100% sequence identity with any one of SEQ ID NO: 1. In some embodiments, the onconase polypeptide is a variant onconase polypeptide.

In some embodiments, the fusion polypeptide comprises a variant onconase polypeptide operably linked to one or more oligopeptides by a peptide bond. In some embodiments, the variant onconase comprises one or more amino acid substitutions as compared to the amino acid sequence of SEQ ID NO: 1. In some embodiments, the fusion protein comprises an onconase polypeptide comprising one or more insertions or deletions as compared to the amino acid sequence of SEQ ID NO: 1. The one or more insertions or deletions may be of any amino acid length.

In some embodiments, the fusion protein comprises an onconase polypeptide with one or more amino acid substitutions that decrease susceptibility to chemical cleavage as compared to an onconase polypeptide having the amino acid sequence of SEQ ID NO: 1. In some embodiments, the fusion protein comprises an onconase polypeptide with one or more deletions or insertions that reduce susceptibility to chemical cleavage as compared to an onconase polypeptide having the amino acid sequence of SEQ ID NO: 1.

In some embodiments, the fusion protein comprises an onconase polypeptide with one or more amino acid substitutions that increase yield or promote formation of inclusion bodies as compared to an onconase polypeptide having the amino acid sequence of SEQ ID NO: 1. In some embodiments, the one or more amino acid substitutions that increase yield or promote formation of inclusion bodies comprise one or more substitutions of cationic or polar amino acids for non-polar or neutral amino acids. In some embodiments, the one or more amino acid substitutions result in a variant onconase polypeptide having a decreased number of cationic or polar amino acids as compared to an onconase polypeptide having the amino acid sequence of SEQ ID NO: 1. In some embodiments, the fusion protein comprises an onconase polypeptide with one or more deletions or insertions that increase yield or promote formation of inclusion bodies as compared to an onconase polypeptide having the amino acid sequence of SEQ ID NO: 1. In some embodiments, the one or more insertions or deletions that increase yield or promote formation of inclusion bodies result in a variant onconase having a decreased number of cationic or polar amino acids as compared to an onconase polypeptide having the amino acid sequence of SEQ ID NO: 1. In some embodiments, the one or more oligopeptides are 4-50 or 5-30 amino acids in length. In some embodiments, the operable linkage comprises an Asp-Pro bond.

In some embodiments, the fusion protein comprises an onconase polypeptide comprising the amino acid sequence of any one of SEQ ID NOs: 2-10 and 15-22 operably linked to one or more oligopeptides, optionally with one, two, three, four, five or all six C-terminal histidine residues deleted as shown in FIG. 20A. In some embodiments, the fusion protein comprises an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to any of SEQ ID NOs: 2-10 and 15-22, optionally with one, two, three, four, five or all six C-terminal histidine residues deleted as shown in FIG. 20A. In some embodiments, the onconase polypeptide comprises an amino acid sequence that has between 80% to 85%, 85% to 90%, 90% to 95%, or 95% to 100% sequence identity to SEQ ID NOs: 2-10 and 15-22, optionally with one, two, three, four, five or all six C-terminal histidine residues deleted as shown in FIG. 20A. In some embodiments, the onconase polypeptide comprises one or more amino acid substitution compared to SEQ ID NO: 1. In some embodiments, the onconase polypeptide comprises one or more deletions or insertions compared to SEQ ID NO: 1. In some embodiments, the one or more oligopeptides are 4-50 or 5-30 amino acids in length. In some embodiments, the operable linkage comprises an Asp-Pro bond.

In some embodiments, the fusion polypeptide comprises an onconase polypeptide with a modified N-terminus and/or C-terminus operably linked to one or more oligopeptides. In some embodiments, the fusion polypeptide comprises an onconase polypeptide one or more amino acid substitution in the N-terminus and/or C-terminus as compared to SEQ ID NO: 1. In some embodiments, the fusion polypeptide comprises an onconase polypeptide comprising 11 amino acid substitutions in the N-terminus and/or C-terminus as compared to SEQ ID NO: 1. In some embodiments, the fusion polypeptide comprises an onconase polypeptide 11 amino acid substitutions in the N-terminus as compared to SEQ ID NO: 1. In some embodiments, the one or more oligopeptides are 4-50 or 5-30 amino acids in length. In some embodiments, the operable linkage comprises an Asp-Pro bond.

In some embodiments, the fusion polypeptide comprises a TAF12 polypeptide comprising the amino acid sequence of SEQ ID NO: 23 operably linked to one or more oligopeptides by a peptide bond. In some embodiments, the fusion polypeptide comprises a TAF12 polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to SEQ ID NO: 23. In some embodiments, the TAF12 polypeptide an amino acid sequence that has between 80% to 85%, 85% to 90%, 90% to 95%, or 95% to 100% sequence identity with any one of SEQ ID NO: 23. In some embodiments, the TAF12 polypeptide is a variant TAF12 polypeptide.

In some embodiments, the fusion polypeptide comprises a variant TAF12 polypeptide operably linked to one or more oligopeptides by a peptide bond. In some embodiments, the variant TAF12 comprises one or more amino acid substitutions as compared to the amino acid sequence of SEQ ID NO: 23. In some embodiments, the fusion protein comprises a TAF12 polypeptide comprising one or more insertions or deletions as compared to the amino acid sequence of SEQ ID NO: 23. The one or more insertions or deletions may be of any amino acid length.

In some embodiments, the fusion protein comprises a TAF12 polypeptide with one or more amino acid substitutions that decrease susceptibility to chemical cleavage as compared to a TAF12 polypeptide having the amino acid sequence of SEQ ID NO: 23. In some embodiments, the fusion protein comprises a TAF12 polypeptide with one or more deletions or insertions that reduce susceptibility to chemical cleavage as compared to a TAF12 polypeptide having the amino acid sequence of SEQ ID NO: 23.

In some embodiments, the fusion protein comprises a TAF12 polypeptide with one or more amino acid substitutions that increase yield or promote formation of inclusion bodies as compared to a TAF12 polypeptide having the amino acid sequence of SEQ ID NO: 23. In some embodiments, the one or more amino acid substitutions that increase yield or promote formation of inclusion bodies comprise one or more substitutions of cationic or polar amino acids for non-polar or neutral amino acids. In some embodiments, the one or more amino acid substitutions result in a variant TAF12 polypeptide having a decreased number of cationic or polar amino acids as compared to a TAF12 polypeptide having the amino acid sequence of SEQ ID NO: 23. In some embodiments, the fusion protein comprises a TAF12 polypeptide with one or more deletions or insertions that increase yield or promote formation of inclusion bodies as compared to a TAF12 polypeptide having the amino acid sequence of SEQ ID NO: 23. In some embodiments, the one or more insertions or deletions that increase yield or promote formation of inclusion bodies result in a variant TAF12 having a decreased number of cationic or polar amino acids as compared to a TAF12 polypeptide having the amino acid sequence of SEQ ID NO: 23. In some embodiments, the one or more oligopeptides are 4-50 or 5-30 amino acids in length. In some embodiments, the operable linkage comprises an Asp-Pro bond.

In some embodiments, the TAF12 polypeptide comprises one or more amino acid substitution compared to SEQ ID NO: 23. In some embodiments, the TAF12 polypeptide comprises one or more deletions or insertions compared to SEQ ID NO: 23. In some embodiments, the one or more oligopeptides are 4-50 or 5-30 amino acids in length. In some embodiments, the operable linkage comprises an Asp-Pro bond.

In some embodiments, the fusion polypeptide comprises a TAF12 polypeptide with a modified N-terminus and/or C-terminus operably linked to one or more oligopeptides. In some embodiments, the fusion polypeptide comprises a TAF12 polypeptide one or more amino acid substitution in the N-terminus and/or C-terminus as compared to SEQ ID NO: 23. In some embodiments, the fusion polypeptide comprises a TAF12 polypeptide comprising 11 amino acid substitutions in the N-terminus and/or C-terminus as compared to SEQ ID NO: 23. In some embodiments, the fusion polypeptide comprises a TAF12 polypeptide 11 amino acid substitutions in the N-terminus as compared to SEQ ID NO: 23. In some embodiments, the one or more oligopeptides are 4-50 or 5-30 amino acids in length. In some embodiments, the operable linkage comprises an Asp-Pro bond.

In some embodiments, the fusion polypeptide is expressed in a cell or cell culture. Any cell or cell culture known to produce inclusion bodies is suitable for expression of the fusion peptide. In some embodiments, the fusion polypeptide is expressed in a bacteria. In some embodiments, the bacteria is a strain of Escherichia coli or Vibrio natriegens. In some embodiments, the bacteria is an E. coli strain. In some embodiments, the bacterial cell is a strain that lacks Lon and ompT protease function (e.g. a BL21 E. coli strain). In some embodiments, the fusion polypeptide is a yeast cell. In some embodiments, the yeast cell is a strain of Pichia pastoris. In some embodiments, the fusion polypeptide is produced by any of the methods disclosed herein.

### VI. Oligopeptides

Provided herein are oligopeptides comprising an active amino acid sequence. In some embodiments, the active amino acid sequence is 4-50 or 5-30 amino acids long. In some embodiments, the active amino acid sequence is a micropeptide (miPEPs). In some embodiments, the oligopeptide comprise an active amino acid sequence and an N-terminal proline, a C-terminal aspartic acid, or both the N-terminal proline and the C-terminal aspartic acid. miPEPs are short peptides (7-44 amino acids long) defined by one or several short open reading frames located in the pri-microRNA sequence of a specific miRNA member. Without being limited by theory, miPEPs are thought to target the microRNA that encodes them at the transcriptional level, modulating the expression of the target pri-microRNA to either upregulate or downregulate transcription. miPEPs may target miRNAs involved various processes, such as miRNAs involved in immunity and susceptibility to a pathogen (e.g., a microbe), organogenesis, response to stress, embryonic development, etc.

In some embodiments, the oligopeptide comprises a miPEP sequence that regulates a miRNA. Each miPEP may regulate one or more members or a particular miRNA family, and in a particular species. The miPEP sequence may regulate a plant miRNA, or a metazoan miRNA. In some embodiments, the miPEP sequence regulates one or more members of a particular miRNA family. In some embodiments, the miPEP sequence regulates a plant miRNA. Exemplary plant microRNA families that may be regulated by miPEPs include, but are not limited to, plant miRNA families miR156, miR159/319, miR160, miR162, miR164, miR166, miR167, miR168, miR169, miR171, miR172, miR390, miR393, miR394, miR295, miR396, miR397, miR398, miR408, miR403, miR437, miR444, and miR445. In some embodiments, the miPEP sequence comprises an N-terminal proline, a C-terminal aspartic acid, or both the N-terminal proline and the C-terminal aspartic acid. In some embodiments, the miPEP sequence has microbe inhibiting activity.

In some embodiments, the oligopeptide comprises an active amino acid sequence that has microbe inhibiting activity. In other embodiments, the oligopeptide comprises a peptide microbial inhibitor. In some embodiments, the oligopeptide inhibits a microbe. In some embodiments, the microbe is a virus, a bacteria, a fungi, an amoeba, or an eukaryote. Exemplary targets of microbes with may be inhibited by the oligopeptides, include, but are not limited to, microbes from the genera Venturia, Podosphaera, Erysiphe, Monolinia, Mycosphaerella, Uncinula, Hemileia, Rhizoctonia, Puccinia, Botrytis, Helminthosporium, Rhynchosporium, Fusarium, Septoria, Cercospora, Alternaria, Pyricularia, Pseudocercosporella, Phytophthora, Peronospora Bremia, Pythium, Plasmopara, Scleropthora, Peronosclerospora, Physopella, Cercospora, Colletotrichum, Gibberell, Exserohilum, Bacillus, Richoderna, Kabateillu, Bipolaris, Pseudomonas, Pseudomonas Erwinia, Mycoplasma, and Rickettsia. In some embodiments, the oligopeptide comprises an active amino acid sequence and an N-terminal proline, a C-terminal aspartic acid, or both the N-terminal proline and the C-terminal aspartic acid.

In some embodiments, the oligopeptide is less than 25 amino acids in length. In some embodiments, the oligopeptide is at least four, at least five, at least six, at least seven, at least eight, at least nine, at least ten, at least fifteen, at least twenty, or at least twenty-five amino acids long. In some embodiments, the oligopeptide is up to fifty amino acids in length. In some embodiments, the oligopeptide is less than fifty, less than forty-five, less than forty, less than thirty-five, less than thirty, less than twenty-five, or less than twenty amino acids long. In some embodiments, the oligopeptide is between four and fifty, between six and forty amino acids long, between six and thirty amino acids long, or between eight and twenty-five amino acids long. In some embodiments, the oligopeptide comprises an active amino acid sequence and an N-terminal proline, a C-terminal aspartic acid, or both the N-terminal proline and the C-terminal aspartic acid.

In some embodiments, the oligopeptide comprises an active amino acid sequence that is 4-50 or 5-30 amino acids long. In some embodiments, the active amino acid sequence is at least four, at least five, at least six, at least seven, at least eight, at least nine, at least ten, at least fifteen, at least twenty, or at least twenty-five amino acids long. In some embodiments, the oligopeptide is up to fifty amino acids in length. In some embodiments, the active amino acid sequence is less than fifty, less than forty-five, less than forty, less than thirty-five, less than thirty, less than twenty-five, or less than twenty amino acids long. In some embodiments, the active amino acid sequence is between four and fifty, between six and forty amino acids long, between six and thirty amino acids long, or between eight and twenty-five amino acids long. In some embodiments, the oligopeptide comprises an active amino acid sequence and a tag comprising additional amino acids. In some embodiments, the tag is a rest of cleavage of a linker. In some embodiments, the tag is located N or C terminal to the active amino acid sequence. In some embodiments the oligopeptide comprises a tag on both the N and C-terminus.

In some embodiments, the oligopeptide comprises the amino acid sequence of any one of SEQ ID NOs: 12-14 and 31-2401. In some embodiments, one or more oligopeptides comprise an amino acid sequence that has at least 80%, at least 85%, at least 90%, at least 91%, at least 92%, at least 93%, at least 94%, at least 95%, at least 96%, at least 97%, at least 98%, or at least 99% sequence identity with the amino acid sequence set forth in SEQ ID NO: 12. In some embodiments, one or more oligopeptides comprise an amino acid sequence that has between 80% to 85%, 85% to 90%, 90% to 95%, or 95% to 100% sequence identity with the amino acid sequence set forth in SEQ ID NO: 13. In some embodiments, the one or more oligopeptides comprise at least one amino acid sequence that has at least 80%, at least 85%, at least 90%, at least 91%, at least 92%, at least 93%, at least 94%, at least 95%, at least 96%, at least 97%, at least 98%, or at least 99% sequence identity with the amino acid sequence set forth in SEQ ID NO: 14. In some embodiments, the oligopeptide is 4-50 or 5-30 amino acids long. In some embodiments, the oligopeptide comprises an active amino acid sequence and an N-terminal proline, a C-terminal aspartic acid, or both the N-terminal proline and the C-terminal aspartic acid.

In some embodiments, the oligopeptides provided herein are produced by cleavage of a concatemer of multiple oligopeptides that are linked together and expressed as a single polypeptide chain. In some embodiments, the cleavage is of a homoconcatemer. In some embodiments, the cleavage is of a heteroconcatemer. In some embodiments, cleavage results in a short (one to two) amino acid tag at the N and/or C terminus of the oligopeptide.

In some embodiments, the oligopeptide comprises an active amino acid sequence and an N-terminus and/or C-terminus residues. The N-terminus and/or C-terminus residues may correspond to a cleavage tag. For instance, the residue may be part of a peptide bond that is cleaved by chemical (e.g. acidic cleavage), or enzymatic reactions. In some embodiments, the oligopeptide comprises an active amino acid sequence and an N-terminal proline, methionine, tryptophan, glycine or cysteine, and/or a C-terminal methionine, aspartic acid, cysteine, asparagine, or tryptophan. In some embodiments, the oligopeptide comprises an active amino acid sequence and an N-terminal proline, a C-terminal aspartic acid, or both the N-terminal proline and the C-terminal aspartic acid. In some embodiments, the oligopeptide is 4-50 or 5-30 amino acids long. In some embodiments, the active amino acid sequence is a miPEP.

### VII. Compositions

Provided herein are compositions comprising one or more oligopeptides produced by any of the methods of the disclosure. In some embodiments, the one or more oligopeptides comprise an active amino acid sequence. In some embodiments, the one or more oligopeptides are 4-50 or 5-30 amino acids long. In some embodiments, the one or more oligopeptides comprise an N-terminal proline, a C-terminal aspartic acid, or both the N-terminal proline and the C-terminal aspartic acid.

In some embodiments, the compositions provided herein are produced by cleavage of a concatemeric polypeptide. In some embodiments, the concatemeric polypeptide is a heteroconcatemer. In some embodiments, the concatemeric polypeptide is a homoconcatemer. In some embodiments, the composition comprises a single type of active oligopeptide produced by cleavage of a homoconcatemer. In some embodiments, the composition comprises multiple active oligopeptides produced by cleavage of a heteroconcatemer. Various ratios of peptides in the composition such as 1:1, 1:2, 1:3, 1:4, 1:1:1 are contemplated.

In some embodiments, the composition is formulated as a liquid, a gel, an emulsion, a suspension, an encapsulation, a solid, a powder, an aerosol, a paste, a coating, a spray, a soil drench, a microcapsule, an emulsifiable concentrate, or as granules. In some embodiments, the composition is formulated as for agricultural use. In some embodiments, the composition is formulated as a seed treatment, a foliar spray, a foliar drench, a Ready-To-Use (RTU) formulation, a produce coating, a suspension concentrate, a tank-mix, an aerosol, a root dip, a soil treatment, a dipping formulation, an irrigation formulation, or a sprinkler formulation. In some embodiments, the composition is formulated to be applied to a plant. In some embodiments, the composition is formulated to be applied to one or more of a seed, root, tuber, fruit, leaf, bulb, rhizome, or flower. In some embodiments, the composition can be applied to a plant by foliar spray, foliar drench, drip irrigation, coating, mixing, pouring, dusting, atomizing, soil irrigation, fumigation, soil injection, seepage irrigation, sprinklers or manual irrigation. In some embodiments, the composition is formulated to be applied to a plant by foliar spray. In some embodiments, the composition formulated for agricultural use comprises one or more oligopeptides. In some embodiments, the one or more oligopeptides are 4-50 or 5-30 amino acids long. In some embodiments, the one or more oligopeptides comprise an N-terminal proline, a C-terminal aspartic acid, or both the N-terminal proline and the C-terminal aspartic acid.

In some embodiments, the composition is formulated as a pharmaceutically acceptable composition. In some embodiments, the pharmaceutically acceptable composition is formulated as a liquid, an emulsion, a liquefied drop, a spray, a foam preparation, a granule, a fine granule, a powder, a capsule, a pill, a paste, a tablet, chewables, injections, suppositories, creams, shampoos, rinses, resins, smokes, or baits. In some embodiments, the pharmaceutically acceptable composition is formulated for administration to a non-human animal. In some embodiments, the pharmaceutically acceptable composition is formulated for administration to a human. In some embodiments, the pharmaceutically acceptable composition comprises one or more oligopeptides. In some embodiments, the one or more oligopeptides are 4-50 or 5-30 amino acids long. In some embodiments, the one or more oligopeptides comprise an N-terminal proline, a C-terminal aspartic acid, or both the N-terminal proline and the C-terminal aspartic acid.

In some embodiments, the composition comprises one or more oligopeptides and a carrier. In some embodiments, the carrier is an agriculturally acceptable carrier. In some embodiments, the agriculturally acceptable carrier comprises a solid carrier, a liquid carrier, a gel carrier, a suspension, or an emulsion. In some embodiments, the agriculturally acceptable carrier comprises an adjuvant, an inert component, a dispersant, a surfactant, an emulsifier, a thickener, a wetting agent, a fertilizer, a mineral, a solvent, a tackifier, a binder, or a stabilizer. In some embodiments, the composition comprises one or more oligopeptides and a pharmaceutically acceptable carrier. In some embodiments, the one or more oligopeptides are 4-50 or 5-30 amino acids long. In some embodiments, the one or more oligopeptides comprise an N-terminal proline, a C-terminal aspartic acid, or both the N-terminal proline and the C-terminal aspartic acid. In some embodiments, the composition comprises one or more oligopeptides oligopeptide comprising an active amino acid sequence. In some embodiments, the active amino acid sequence is a micropeptide (miPEPs). In some embodiments, the oligopeptide comprises a miPEP sequence that regulates a miRNA. In some embodiments, the miPEP sequence regulates one or more members of a particular miRNA family. In some embodiments, the miPEP sequence regulates a plant miRNA. Exemplary plant microRNA families that may be regulated by miPEPs include, but are not limited to, plant miRNA families miR156, miR159/319, miR160, miR162, miR164, miR166, miR167, miR168, miR169, miR171, miR172, miR390, miR393, miR394, miR295, miR396, miR397, miR398, miR408, miR403, miR437, miR444, and miR445. In some embodiments, the composition comprises a miPEP that has microbe inhibiting activity. In some embodiments, the composition comprises a miPEP that inhibits a virus, a bacteria, a fungi, an amoeba, or an eukaryote. In some embodiments, the miPEPs comprise an N-terminal proline, a C-terminal aspartic acid, or both the N-terminal proline and the C-terminal aspartic acid. In some embodiments, the one or more oligopeptides are 4-50 or 5-30 amino acids long.

In some embodiments, the composition comprises one or more oligopeptides comprising the amino acid sequence of any one of SEQ ID NOs: 12-14 and 31-2401. In some embodiments, one or more oligopeptides comprise an amino acid sequence that has at least 80%, at least 85%, at least 90%, at least 91%, at least 92%, at least 93%, at least 94%, at least 95%, at least 96%, at least 97%, at least 98%, or at least 99% sequence identity with the amino acid sequence set forth in SEQ ID NO: 12. In some embodiments, one or more oligopeptides comprise an amino acid sequence that has between 80% to 85%, 85% to 90%, 90% to 95%, or 95% to 100% sequence identity with the amino acid sequence set forth in SEQ ID NO: 13. In some embodiments, the one or more oligopeptides comprise at least one amino acid sequence that has at least 80%, at least 85%, at least 90%, at least 91%, at least 92%, at least 93%, at least 94%, at least 95%, at least 96%, at least 97%, at least 98%, or at least 99% sequence identity with the amino acid sequence set forth in SEQ ID NO: 14. In some embodiments, the composition comprises two or more oligopeptides. In some embodiments, the composition comprises three or more oligopeptides.

In some embodiments, the composition comprises one or more oligopeptides comprise an active amino acid sequence and an N-terminus and/or C-terminus residues. The N-terminus and/or C-terminus residues may correspond to a cleavage tag. In some embodiments, the oligopeptide comprises an active amino acid sequence and an N-terminal proline, methionine, tryptophan, glycine or cysteine, and/or a C-terminal methionine, aspartic acid, cysteine, asparagine, or tryptophan. In some embodiments, the composition comprises one or more oligopeptides comprising an active amino acid sequence and an N-terminal praline, a C-terminal aspartic acid, or both the N-terminal praline and the C-terminal aspartic acid.

### VII. Nucleic Acids

Also provided are nucleic acids encoding any of the fusion polypeptides, polypeptides, insoluble carrier polypeptides (e.g., onconase polypeptides or TAF12 polypeptides) or oligopeptides disclosed herein. In some embodiments, the nucleic acid includes sequence elements that enhance the expression of the encoded polypeptide in a cell. For examples, the nucleic acid may comprise an origin of replication that when incorporated into a vector leads to a copy number that is high enough to yield expression of the encoded polypeptide. In some embodiments, the nucleic acid comprises a promoter that regulates the expression of the fusion polypeptide. In some embodiments, the promoter is a bacterial or yeast promoter. In some embodiments, the promoter is a constitutive or an inducible promoter. In some embodiments, the promoter is a strong promoter. In some embodiments, the promoter drives the expression of the encoded polypeptide in inclusion bodies.

The nucleic acids of the disclosure may be prepared using any technique known in the art. Without limitation, these may include cloning, DNA isolation, amplification and purification, enzymatic reactions involving DNA ligase, DNA polymerase, restriction endonucleases and the like, and various separation techniques, such as gel electrophoresis and chromatography. Several standard techniques are described in Ausubel et al. (1992) Current Protocols in Molecular Biology, Green/Wiley, New York, N.Y.; Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth. Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; Setlow and Hollaender (1979) Genetic Engineering: Principles and Methods, Vols. 1-4, Plenum Press, New York; and Ausubel et al. (1992) Current Protocols in Molecular Biology, Greene/Wiley, New York, N.Y. Abbreviations and nomenclature, where employed, are deemed standard in the field and commonly used in professional journals such as those cited herein.

Also provided herein are vectors comprising nucleic acids encoding any of the fusion polypeptides, insoluble carrier polypeptides (e.g., onconase polypeptides or TAF12 polypeptides) or oligopeptides disclosed herein.

A “vector” is a nucleic acid that is capable of transporting another nucleic acid. Vectors may be, for example, plasmids, viruses, cosmids or phage. An “expression vector” is a vector that is capable of directing expression of a protein encoded by one or more genes carried by the vector when it is present in the appropriate environment. Examples of vectors are those that can autonomously replicate and express structural gene products present in the DNA segments to which they are operatively linked. Vectors, therefore, can contain the replicons and selectable markers described earlier. Vectors include, but are not necessarily limited to, expression vectors. In some embodiments, the vector is a bacterial vector. In some embodiments, the vector is a yeast vector. In some embodiments,

Examples of expression vectors that can be used in prokaryotic host cells include those derived from commercially available plasmids such as the cloning vector pET plasmids (Novagen, Madison, Wis., USA) or pBR322 (ATCC 37017). The pBR322 vector contains genes for ampicillin and tetracycline resistance and thus provides simple means for identifying transformed cells. To construct an expression vector using pBR322, an appropriate promoter and a DNA sequence encoding one or more of the polypeptides of the invention are inserted into the pBR322 vector. Other commercially available vectors include, for example, pKK223-3 (Pharmacia Fine Chemicals, Uppsala, Sweden) and pGEM-1 (Promega Biotec, Madison, Wis., USA). Other commercially available vectors include those that are specifically designed for the expression of proteins; these would include pMAL-p2 and pMAL-c2 vectors that are used for the expression of proteins fused to maltose binding protein (New England Biolabs, Beverly, Mass., USA).

In yeast, a number of vectors containing constitutive or inducible promoters may be used. For a review, see Current Protocols in Molecular Biology, Vol. 2, Ed. Ausubel et al., Greene Publish. Assoc. & Wiley Interscience, Ch. 13 (1988); Bitter et al., Expression and Secretion Vectors for Yeast, in Methods in Enzymology, Eds. Wu & Grossman, 31987, Acad. Press, N.Y., Vol. 153, pp. 516-544 (1987); Glover, DNA Cloning, Vol. II, IRL Press, Wash., D.C., Ch. 3 (1986); Bitter, Heterologous Gene Expression in Yeast, Methods in Enzymology, Eds. Berger & Kimmel, Acad. Press, N.Y., Vol. 152, pp. 673-684 1987); and The Molecular Biology of the Yeast Saccharomyces, Eds. Strathern et al., Cold Spring Harbor Press, Vols. I and II (1982). A constitutive yeast promoter such as ADH1 or LEU2 or an inducible promoter such as GAL4 may be used (Cloning in Yeast, Ch. 3, R. Rothstein In: DNA Cloning Vol. 11, A Practical Approach, Ed. D M Glover, IRL Press, Wash., D.C. (1986)). Alternatively, vectors may be used which promote integration of foreign DNA sequences into the yeast or bacterial chromosome.

Promoter sequences commonly used for recombinant prokaryotic host cell expression vectors include the bacteriophage T7 promoter (Studier and Moffatt, J. Mol. Biol. 189:113 (1986)), β-lactamase (penicillinase), lactose promoter system (Chang et al., Nature 275:615, 1978; Goeddel et al., Nature 281:544 (1979)), tryptophan (tap) promoter system (Goeddel et al., Nucl. Acids Res. 8:4057 (1980); EP-A-36776), and tac promoter (Maniatis, Molecular Cloning: A Laboratory Manual, Cold Spring Harbor Laboratory; p. 412 (1982)). A particularly useful prokaryotic host cell expression system employs a phage λ PL promoter and a c1857ts thermolabile repressor sequence. Plasmid vectors available from the American Type Culture Collection (ATCC), which incorporate derivatives of the PL promoter, include plasmid pHUB2 (resident in E. coli strain JMB9 (ATCC 37092)) and pPLc28 (resident in E. coli RR1 (ATCC 53082)).

In some embodiments, the nucleic acid encodes a fusion polypeptide comprising a protein that forms inclusion bodies in a cell operably linked to one or more oligopeptides by a peptide bond. In some embodiments, the inclusion body tag is an onconase polypeptide. In some embodiments, the protein that forms inclusion bodies in a cell is an onconase. In some embodiments, the onconase is a variant onconase.

In some embodiments, the nucleic acid encodes a fusion polypeptide comprising an insoluble carrier polypeptide (e.g., an onconase polypeptide or a TAF12 polypeptide) operably linked to one or more polypeptides by a peptide bond. In some embodiments, the nucleic acid encodes a fusion polypeptide comprises two or more, three or more oligopeptides, four or more oligopeptides, five or more oligopeptides, six or more polypeptides, eight or more polypeptides, ten or more polypeptides, fifteen or more polypeptides, or twenty or more oligopeptides operably linked to the insoluble carrier polypeptide (e.g., an onconase polypeptide or a TAF12 polypeptide). In some embodiments, the one or more oligopeptides are operably linked to the N-terminus and/or the C-terminus of the onconase. In some embodiments, the one or more oligopeptides are 4-50 or 5-30 amino acids long. In some embodiments, the fusion polypeptide encoded by the nucleic acid comprises a cleavable Asp-Pro bond.

In some embodiments, the nucleic acid encodes a fusion polypeptide comprising an insoluble carrier polypeptide (e.g., an onconase polypeptide or a TAF12 polypeptide) operably linked to one or more polypeptides by a peptide bond that is capable of sequence-specific chemical cleavage. In some embodiments, the peptide bond includes (i) a methionine and the sequence-specific chemical cleavage is with cyanogen bromide, (ii) a tryptophan and the sequence-specific chemical cleavage is with BNPS-skatole, (iii) an aspartic acid-proline (Asp-Pro) bond and the sequence-specific chemical cleavage is with formic acid, (iv) an asparagine-glycine bond and the sequence-specific chemical cleavage is with hydroxylamine, or (v) a cysteine and the sequence-specific chemical cleavage is with NTCB. In some embodiments, the one or more oligopeptides are operably linked to the insoluble carrier polypeptide (e.g., an onconase polypeptide or a TAF12 polypeptide) by a peptide bond comprising an Asp-Pro bond and the sequence-specific chemical cleavage is with formic acid or acetic acid.

In some embodiments, the nucleic acid encodes a fusion polypeptide comprising one or more oligopeptides that are less than 25 amino acids in length. In some embodiments, the one or more oligopeptides are at least four, at least five, at least six, at least seven, at least eight, at least nine, at least ten, at least fifteen, at least twenty, or at least twenty-five amino acids long. In some embodiments, the one or more oligopeptides are up to fifty amino acids in length. In some embodiments, the one or more oligopeptides are less than fifty, less than forty-five, less than forty, less than thirty-five, less than thirty, less than twenty-five, or less than twenty amino acids long. In some embodiments, the one or more oligopeptides are between four and fifty, between six and forty amino acids long, between six and thirty amino acids long, or between eight and twenty-five amino acids long.

In some embodiments, the nucleic acid encodes a fusion polypeptide comprising an onconase operably linked to one or more oligopeptides having the amino acid sequence of any one of SEQ ID NOs: 12-14 and 31-2401. In some embodiments, the one or more oligopeptides comprise an amino acid sequence that has at least 80%, at least 85%, at least 90%, at least 91%, at least 92%, at least 93%, at least 94%, at least 95%, at least 96%, at least 97%, at least 98%, or at least 99% sequence identity with any one of SEQ ID NOs: 12-14 and 31-2401. In some embodiments, the one or more oligopeptides comprise an amino acid sequence that has between 80% to 85%, 85% to 90%, 90% to 95%, or 95% to 100% sequence identity with any one of SEQ ID NOs: 12-14 and 31-2401. In some embodiments, the one or more oligopeptides comprise at least one of the amino acid sequence of any one of SEQ ID NOs: 12-14 and 31-2401. In some embodiments, the one or more oligopeptides comprise at least one amino acid sequence that has at least 80%, at least 85%, at least 90%, at least 91%, at least 92%, at least 93%, at least 94%, at least 95%, at least 96%, at least 97%, at least 98%, or at least 99% sequence identity with any one of SEQ ID NOs: 12-14 and 31-2401. In some embodiments, the one or more oligopeptides comprise at least one amino acid sequence that has between 80% to 85%, 85% to 90%, 90% to 95%, or 95% to 100% sequence identity with any one of SEQ ID NOs: 12-14 and 31-2401. In some embodiments, the onconase is a variant onconase. In some embodiments, the one or more oligopeptides comprise an active amino acid sequence and an N-terminal proline, a C-terminal aspartic acid, or both the N-terminal proline and the C-terminal aspartic acid when released from the fusion polypeptide. In some embodiments, the nucleic acid further comprises a linker sequence between the one or more oligopeptides and/or between the insoluble carrier polypeptide (e.g., an onconase polypeptide or a TAF12 polypeptide) and the oligopeptides. In some embodiments, the linker sequence is capable of being cleaved by sequence-specific chemical cleavage.

In some embodiments, the nucleic acid encodes a fusion polypeptide comprising an insoluble carrier polypeptide (e.g., an onconase polypeptide or a TAF12 polypeptide) operably linked to one or more micropeptides (miPEPs). In some embodiments, the one or more oligopeptides comprise a miPEP sequence that regulates a miRNA. In some embodiments, the one or more oligopeptides comprise a miPEP sequence that regulates one or more miRNA families. In some embodiments, the one or more oligopeptides comprise a miPEP sequence that regulates a plant miRNA. In some embodiments, the one or more oligopeptides comprise a miPEP sequence that has microbe inhibiting activity.

In some embodiments, the nucleic acid encodes a fusion polypeptide comprising an onconase polypeptide comprising the amino acid sequence of SEQ ID NO: 1 operably linked to one or more oligopeptides by a peptide bond. In some embodiments, the onconase polypeptide comprises an amino acid sequence that has at least 80%, at least 85%, at least 90%, at least 91%, at least 92%, at least 93%, at least 94%, at least 95%, at least 96%, at least 97%, at least 98%, or at least 99% sequence identity to SEQ ID NO: 1. In some embodiments, the onconase polypeptide comprises an amino acid sequence that has between 80% to 85%, 85% to 90%, 90% to 95%, or 95% to 100% sequence identity with any one of SEQ ID NO: 1. In some embodiments, the onconase polypeptide comprises one or more amino acid substitutions as compared to the amino acid sequence of SEQ ID NO: 1. In some embodiments, the onconase polypeptide comprises one or more insertions or deletions as compared to the amino acid sequence of SEQ ID NO: 1.

In some embodiments, the nucleic acid encodes a fusion polypeptide comprising an onconase polypeptide with one or more amino acid substitutions that decrease susceptibility to chemical cleavage as compared to an onconase polypeptide having the amino acid sequence of SEQ ID NO: 1. In some embodiments, the nucleic acid encodes a fusion polypeptide comprising an onconase polypeptide with one or more deletions or insertions that reduce susceptibility to chemical cleavage as compared to an onconase polypeptide having the amino acid sequence of SEQ ID NO: 1.

In some embodiments, the nucleic acid encodes a fusion polypeptide comprising an onconase polypeptide with one or more amino acid substitutions, or one or more deletions and insertions that increase yield or promote formation of inclusion bodies as compared to an onconase polypeptide having the amino acid sequence of SEQ ID NO: 1. In some embodiments, the nucleic acid encodes a fusion polypeptide comprising an onconase polypeptide comprising the amino acid sequence of any one of SEQ ID NOs: 2-10, optionally with one, two, three, four, five or all six C-terminal histidine residues deleted as shown in FIG. 20A. In some embodiments, the nucleic acid encodes a fusion polypeptide comprising an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to any of SEQ ID NOs: 2-10 and 15-22, optionally with one, two, three, four, five or all six C-terminal histidine residues deleted as shown in FIG. 20A. In some embodiments, the onconase polypeptide comprises an amino acid sequence that has between 80% to 85%, 85% to 90%, 90% to 95%, or 95% to 100% sequence identity with any one of SEQ ID NOs: 2-10 and 15-22, optionally with one, two, three, four, five or all six C-terminal histidine residues deleted as shown in FIG. 20A.

In some embodiments, the nucleic acid encodes a fusion polypeptide comprising an onconase polypeptide with a modified N-terminus and/or C-terminus operably linked to one or more oligopeptides. In some embodiments, the onconase polypeptide comprises one or more amino acid substitution in the N-terminus and/or C-terminus as compared to SEQ ID NO: 1. In some embodiments, the nucleic acid encodes a fusion polypeptide comprising an onconase polypeptide comprising 11 amino acid substitutions in the N-terminus and/or C-terminus as compared to SEQ ID NO: 1. In some embodiments, the nucleic acid encodes a fusion polypeptide comprising an onconase polypeptide 11 amino acid substitutions in the N-terminus as compared to SEQ ID NO: 1. In some embodiments, the nucleic acid further comprises a linker sequence between the one or more oligopeptides and/or between the onconase polypeptide and the oligopeptides. In some embodiments, the linker sequence is capable of being cleaved by sequence-specific chemical cleavage.

In some embodiments, the nucleic acid encodes a fusion polypeptide comprising a TAF12 polypeptide comprising the amino acid sequence of SEQ ID NO: 23 operably linked to one or more oligopeptides by a peptide bond. In some embodiments, the TAF12 polypeptide comprises an amino acid sequence that has at least 80%, at least 85%, at least 90%, at least 91%, at least 92%, at least 93%, at least 94%, at least 95%, at least 96%, at least 97%, at least 98%, or at least 99% sequence identity to SEQ ID NO: 1. In some embodiments, the TAF12 polypeptide comprises an amino acid sequence that has between 80% to 85%, 85% to 90%, 90% to 95%, or 95% to 100% sequence identity with any one of SEQ ID NO: 23. In some embodiments, the TAF12 polypeptide comprises one or more amino acid substitutions as compared to the amino acid sequence of SEQ ID NO: 23. In some embodiments, the TAF12 polypeptide comprises one or more insertions or deletions as compared to the amino acid sequence of SEQ ID NO: 23.

In some embodiments, the nucleic acid encodes a fusion polypeptide comprising a TAF12 polypeptide with one or more amino acid substitutions that decrease susceptibility to chemical cleavage as compared to a TAF12 polypeptide having the amino acid sequence of SEQ ID NO: 23. In some embodiments, the nucleic acid encodes a fusion polypeptide comprising a TAF12 polypeptide with one or more deletions or insertions that reduce susceptibility to chemical cleavage as compared to a TAF12 polypeptide having the amino acid sequence of SEQ ID NO: 23.

In some embodiments, the nucleic acid encodes a fusion polypeptide comprising a TAF12 polypeptide with one or more amino acid substitutions, or one or more deletions and insertions that increase yield or promote formation of inclusion bodies as compared to a TAF12 polypeptide having the amino acid sequence of SEQ ID NO: 23.

In some embodiments, the nucleic acid encodes a fusion polypeptide comprising a TAF12 polypeptide with a modified N-terminus and/or C-terminus operably linked to one or more oligopeptides. In some embodiments, the TAF12 polypeptide comprises one or more amino acid substitution in the N-terminus and/or C-terminus as compared to SEQ ID NO: 23. In some embodiments, the nucleic acid encodes a fusion polypeptide comprising a TAF12 polypeptide comprising 11 amino acid substitutions in the N-terminus and/or C-terminus as compared to SEQ ID NO: 23. In some embodiments, the nucleic acid encodes a fusion polypeptide comprising a TAF12 polypeptide 11 amino acid substitutions in the N-terminus as compared to SEQ ID NO: 23. In some embodiments, the nucleic acid further comprises a linker sequence between the one or more oligopeptides and/or between the TAF12 polypeptide and the oligopeptides. In some embodiments, the linker sequence is capable of being cleaved by sequence-specific chemical cleavage.

In some embodiments, the nucleic acid encodes an oligopeptide. In some embodiments, the nucleic acid encodes an oligopeptide comprising an active amino acid sequence. In some embodiments, the active amino acid sequence is a micropeptide (miPEPs). In some embodiments, the nucleic acid encodes an oligopeptide comprising a miPEP sequence that regulates a miRNA. In some embodiments, the miPEP sequence regulates one or more members of a particular miRNA family. In some embodiments, the miPEP sequence regulates a plant miRNA. In some embodiments, the miPEP sequence regulates a plant miRNA family selected from the group consisting on miR156, miR159/319, miR160, miR162, miR164, miR166, miR167, miR168, miR169, miR171, miR172, miR390, miR393, miR394, miR295, miR396, miR397, miR398, miR408, miR403, miR437, miR444, and miR445. In some embodiments, the miPEPs comprise an N-terminal proline, a C-terminal aspartic acid, or both the N-terminal proline and the C-terminal aspartic acid.

In some embodiments, the nucleic acid encodes a miPEP that has microbe inhibiting activity.

In some embodiments, the nucleic acid encodes an oligopeptide comprising the amino acid sequence of any one of SEQ ID NOs: 12-14 and 31-2401. In some embodiments, the nucleic acid encodes an oligopeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity with any one of SEQ ID NOs: 12-14 and 31-2401. In some embodiments, the nucleic acid encodes an oligopeptide comprising an amino acid sequence that has between 80% to 85%, 85% to 90%, 90% to 95%, or 95% to 100% sequence identity with any one of SEQ ID NOs: 12-14 and 31-2401. In some embodiments, the nucleic acid encodes an oligopeptide comprising at least one of the amino acid sequence of any one of SEQ ID NOs: 12-14 and 31-2401. In some embodiments, the nucleic acid encodes an oligopeptide comprising at least one amino acid sequence that has at least 80%, at least 85%, at least 90%, at least 91%, at least 92%, at least 93%, at least 94%, at least 95%, at least 96%, at least 97%, at least 98%, or at least 99% sequence identity with any one of SEQ ID NOs: 12-14 and 31-2401. In some embodiments, the nucleic acid encodes an oligopeptide comprising at least one amino acid sequence that has between 80% to 85%, 85% to 90%, 90% to 95%, or 95% to 100% sequence identity with any one of SEQ ID NOs: 12-14 and 31-2401. In some embodiments, the oligopeptide is 4-50 or 5-30 amino acids long. In some embodiments, the oligopeptide comprises an N-terminal proline, a C-terminal aspartic acid, or both the N-terminal proline and the C-terminal aspartic acid.

In some embodiments, the nucleic acid encodes an onconase polypeptide. In some embodiments, the onconase is a variant onconase. In some embodiments, the nucleic acid encodes an onconase polypeptide comprising the amino acid sequence of SEQ ID NO: 1. In some embodiments, the nucleic acid encodes an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to SEQ ID NO: 1. In some embodiments, the nucleic acid encodes an onconase polypeptide comprising an amino acid sequence that has between 80% to 85%, 85% to 90%, 90% to 95%, or 95% to 100% sequence identity to SEQ ID NO: 1. In some embodiments, the nucleic acid encodes an onconase polypeptide comprising one or more amino acid substitutions compared to the amino acid sequence of SEQ ID NO: 1. In some embodiments, the nucleic acid encodes an onconase polypeptide comprising one or more insertions or deletions as compared to the amino acid sequence of SEQ ID NO: 1. In some embodiments, the one or more amino acid substitutions, or one or more deletions or insertions decrease susceptibility of the onconase polypeptide to chemical cleavage as compared to an onconase polypeptide having the amino acid sequence of SEQ ID NO: 1. In some embodiments, the one or more amino acid substitutions, or one or more insertions or deletions result in a variant onconase that has increased yield or increase capacity to form inclusion bodies in a cell than an onconase polypeptide having the amino acid sequence of SEQ ID NO: 1. In some embodiments, the nucleic acid encodes an onconase polypeptide comprising the amino acid sequence of any one of SEQ ID NOs: 2-10 and 15-22, optionally with one, two, three, four, five or all six C-terminal histidine residues deleted as shown in FIG. 20A. In some embodiments, the nucleic acid encodes an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to any of SEQ ID NOs: 2-10 and 15-22, optionally with one, two, three, four, five or all six C-terminal histidine residues deleted as shown in FIG. 20A. In some embodiments, the nucleic acid encodes an onconase polypeptide having at least one amino acid sequence that has between 80% to 85%, 85% to 90%, 90% to 95%, or 95% to 100% sequence identity with any one of SEQ ID NOs: 12-14 and 31-2401.

In some embodiments, the nucleic acid encodes an onconase polypeptide comprising a modified N-terminus and/or C-terminus. In some embodiments, the nucleic acid encodes an onconase polypeptide comprising one or more amino acid substitution in the N-terminus and/or C-terminus as compared to SEQ ID NO: 1. In some embodiments, the nucleic acid encodes an onconase polypeptide comprising 11 amino acid substitutions in the N-terminus and/or C-terminus as compared to SEQ ID NO: 1. In some embodiments, the nucleic acid encodes an onconase polypeptide comprising 11 amino acid substitutions in the N-terminus as compared to SEQ ID NO: 1.

In some embodiments, the nucleic acid encodes a TAF12 polypeptide. In some embodiments, the TAF12 is a variant TAF12. In some embodiments, the nucleic acid encodes a TAF12 polypeptide comprising the amino acid sequence of SEQ ID NO: 23. In some embodiments, the nucleic acid encodes a TAF12 polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to SEQ ID NO: 23. In some embodiments, the nucleic acid encodes a TAF12 polypeptide comprising an amino acid sequence that has between 80% to 85%, 85% to 90%, 90% to 95%, or 95% to 100% sequence identity to SEQ ID NO: 23. In some embodiments, the nucleic acid encodes a TAF12 polypeptide comprising one or more amino acid substitutions compared to the amino acid sequence of SEQ ID NO: 23. In some embodiments, the nucleic acid encodes a TAF12 polypeptide comprising one or more insertions or deletions as compared to the amino acid sequence of SEQ ID NO: 23. In some embodiments, the one or more amino acid substitutions, or one or more deletions or insertions decrease susceptibility of the TAF12 polypeptide to chemical cleavage as compared to a TAF12 polypeptide having the amino acid sequence of SEQ ID NO: 23. In some embodiments, the one or more amino acid substitutions, or one or more insertions or deletions result in a variant TAF12s that has increased yield or increase capacity to form inclusion bodies in a cell than a TAF12 polypeptide having the amino acid sequence of SEQ ID NO: 23. In some embodiments, the nucleic acid encodes a TAF12 polypeptide having at least one amino acid sequence that has between 80% to 85%, 85% to 90%, 90% to 95%, or 95% to 100% sequence identity with any one of SEQ ID NOs: 12-14 and 31-2401.

In some embodiments, the nucleic acid encodes a TAF12 polypeptide comprising a modified N-terminus and/or C-terminus. In some embodiments, the nucleic acid encodes a TAF12 polypeptide comprising one or more amino acid substitution in the N-terminus and/or C-terminus as compared to SEQ ID NO: 23. In some embodiments, the nucleic acid encodes a TAF12 polypeptide comprising 11 amino acid substitutions in the N-terminus and/or C-terminus as compared to SEQ ID NO: 23. In some embodiments, the nucleic acid encodes a TAF12 polypeptide comprising 11 amino acid substitutions in the N-terminus as compared to SEQ ID NO: 23.

In some embodiments, the nucleic acid encodes an insoluble carrier polypeptide (e.g., an onconase polypeptide or a TAF12 polypeptide) comprising an amino acid sequence tag. In some embodiments, the amino acid sequence tag is a purification amino acid sequence tag. In some embodiments, the amino acid sequence tag is a detection amino acid sequence tag. In some embodiments, the onconase is a variant onconase.

### IX. Cells

Also provided are cells expressing any of the fusion polypeptides, oligopeptides, tagged miPEPs or insoluble carrier polypeptides (e.g., onconase polypeptides or TAF12 polypeptides) described herein. In some embodiment, the cell is a bacterial or yeast cell. In some embodiments, the cell is a host cell.

In some embodiments, the cell is a bacteria cell, a yeast cell, a plant cell, or a metazoan cell. In some embodiments, the cell is a bacterial cell. In some embodiments, the bacteria is an E. coli strain. In some embodiments, the cell is a cell that is capable of forming inclusion bodies. In some embodiments, the cell is a prokaryotic cell. In some embodiments, the cell is a eukaryotic cell. In some embodiments, the cell is a fungal cell. In some embodiments, the cell is an algal cell.

Microorganism host cells useful in the present invention may include, but are not limited to, bacteria, such as the enteric bacteria (Escherichia and Salmonella for example) as well as Bacillus, Acinetobacter, Streptomyces, Methylobacter, Rhodococcus and Pseudomonas; Cyanobacteria, such as Rhodobacter and Synechocystis; yeasts, such as Saccharomyces, Zygosaccharomyces, Kluyveromyces, Candida, Hansenula, Debaryomyces, Mucor, Pichia, Yarrowia, and Torulopsis; and filamentous fungi such as Aspergillus and Arthrobotrys, and algae for example.

In some embodiments, the cell comprises one or more modifications to increase expression of the fusion protein. In some embodiments, the cell comprises mutations or deletions of one or more proteases. In some embodiments, the cell comprises a mutation or deletion in an OmpT or Lon gene.

In some embodiments, the host cell comprises at least one copy of a nucleic acid sequence encoding a fusion polypeptide. The at least one copy of the nucleic acid sequence encoding a fusion polypeptide enzyme can be present in the chromosome of a prokaryotic (bacterial) cell or in one chromosome of a eukaryotic cell. Alternatively, the at least one copy of the nucleic acid sequence encoding a fusion polypeptide can be present in a vector or plasmid that is present in the cell. The host cell, as described above, can be a prokaryotic or eukaryotic cell. If it is a prokaryotic cell, it can be a bacterial cell. If it is a eukaryotic cell, it can be a yeast cell, a plant cell, or an animal cell. Suitable host cells are described herein.

In some embodiments, the cell expresses a fusion polypeptide comprising an insoluble carrier polypeptide, which forms inclusion bodies operably linked to one or more oligopeptides. In some embodiments, the insoluble carrier polypeptide, which forms inclusion bodies is an insoluble carrier polypeptide (e.g., an onconase polypeptide or a TAF12 polypeptide). In some embodiments, the insoluble carrier polypeptide (e.g., an onconase polypeptide or a TAF12 polypeptide) is a variant insoluble carrier polypeptide (e.g., a variant onconase polypeptide or a variant TAF12 polypeptide). In some embodiments, the one or more oligopeptides are 4-50 or 5-30 amino acids in length. In some embodiments, the one or more oligopeptides are operably linked to the N-terminus and/or the C-terminus of the insoluble carrier polypeptide (e.g., the onconase polypeptide or the TAF12 polypeptide). In some embodiments, the operable linkage comprises an Asp-Pro bond.

In some embodiments, the cell expresses a fusion polypeptide comprising an insoluble carrier polypeptide (e.g., an onconase polypeptide or a TAF12 polypeptide) operably linked to one or more oligopeptides comprise the amino acid sequence of any one of SEQ ID NOs: 12-14 and 31-2401. In some embodiments, the one or more oligopeptides comprise an amino acid sequence that has at least 80%, at least 85%, at least 90%, at least 91%, at least 92%, at least 93%, at least 94%, at least 95%, at least 96%, at least 97%, at least 98%, or at least 99% sequence identity with any one of SEQ ID NOs: 12-14 and 31-2401. In some embodiments, the one or more oligopeptides comprise an amino acid sequence that has between 80% to 85%, 85% to 90%, 90% to 95%, or 95% to 100% sequence identity with any one of SEQ ID NOs: 12-14 and 31-2401. In some embodiments, the one or more oligopeptides comprise at least one of the amino acid sequence of any one of SEQ ID NOs: 12-14 and 31-2401. In some embodiments, the one or more oligopeptides comprise at least one amino acid sequence that has at least 80%, at least 85%, at least 90%, at least 91%, at least 92%, at least 93%, at least 94%, at least 95%, at least 96%, at least 97%, at least 98%, or at least 99% sequence identity with any one of SEQ ID NOs: 12-14 and 31-2401. In some embodiments, the one or more oligopeptides comprise at least one amino acid sequence that has between 80% to 85%, 85% to 90%, 90% to 95%, or 95% to 100% sequence identity with any one of SEQ ID NOs: 12-14 and 31-2401. In some embodiments, the one or more oligopeptides comprise an active amino acid sequence and an N-terminal proline, a C-terminal aspartic acid, or both the N-terminal proline and the C-terminal aspartic acid when released from the fusion polypeptide. In some embodiments, the insoluble carrier polypeptide (e.g., the onconase polypeptide or the TAF12 polypeptide) is a variant insoluble carrier polypeptide (e.g., a variant onconase polypeptide or a variant TAF12 polypeptide). In some embodiments, the operable linkage comprises an Asp-Pro bond.

In some embodiments, the cell expresses a fusion polypeptide comprising an insoluble carrier polypeptide (e.g., an onconase polypeptide or a TAF12 polypeptide) operably linked to one or more micropeptides (miPEPs). In some embodiments, the one or more oligopeptides comprise a miPEP sequence that regulates an miRNA. In some embodiments, the one or more oligopeptides comprise a miPEP sequence that regulates one or more members of an miRNA family. In some embodiments, the one or more oligopeptides comprise a miPEP sequence that regulates a plant miRNA. In some embodiments, the one or more oligopeptides comprise a miPEP sequence that regulates a plant miRNA selected from the group consisting of the miR156, miR159/319, miR160, miR162, miR164, miR166, miR167, miR168, miR169, miR171, miR172, miR390, miR393, miR394, miR295, miR396, miR397, miR398, miR408, miR403, miR437, miR444, and miR445 families. In some embodiments, the one or more oligopeptides comprise a miPEP sequence that has microbe inhibiting activity. In some embodiments, the insoluble carrier polypeptide (e.g., the onconase polypeptide or the TAF12 polypeptide) is a variant insoluble carrier polypeptide (e.g., a variant onconase polypeptide or a variant TAF12 polypeptide). In some embodiments, the operable linkage comprises an Asp-Pro bond.

In some embodiments, the cell expresses a fusion polypeptide further comprising a linker sequence between the one or more oligopeptides and/or between the insoluble carrier polypeptide (e.g., an onconase polypeptide or a TAF12 polypeptide) and the oligopeptides. In some embodiments, the linker sequence separates the one or more oligopeptides and/or a linker sequence separating the insoluble carrier polypeptide (e.g., an onconase polypeptide or a TAF12 polypeptide) from the one or more oligopeptides. In some embodiments, the linker sequence separating the one or more oligopeptides has the same or different sequence than the linker sequence separating the insoluble carrier polypeptide (e.g., an onconase polypeptide or a TAF12 polypeptide) from the one or more oligopeptides. In some embodiments, the cell expresses a fusion polypeptide further comprising one or more amino acid sequence tag.

In some embodiments, the cell expresses a fusion polypeptide comprising an insoluble carrier polypeptide comprising an onconase polypeptide. In some embodiments, the onconase polypeptide comprises the amino acid sequence of SEQ ID NO: 1 operably linked to one or more oligopeptides by a peptide bond. In some embodiments, the onconase polypeptide comprises an amino acid sequence that has at least 80%, at least 85%, at least 90%, at least 91%, at least 92%, at least 93%, at least 94%, at least 95%, at least 96%, at least 97%, at least 98%, or at least 99% sequence identity to SEQ ID NO: 1. In some embodiments, the onconase polypeptide comprises an amino acid sequence that has between 80% to 85%, 85% to 90%, 90% to 95%, or 95% to 100% sequence identity to SEQ ID NO: 1. In some embodiments, the onconase polypeptide comprises one or more amino acid substitutions, or one or more insertions or deletions, as compared to the amino acid sequence of SEQ ID NO: 1. In some embodiments, the one or more amino acid substitutions, or one or more insertions or deletions, decrease susceptibility of the onconase to chemical cleavage as compared to an onconase polypeptide having the amino acid sequence of SEQ ID NO: 1. In some embodiments, the one or more amino acid substitutions, or one or more insertion or deletions results in an onconase polypeptide that has increase yield or higher capacity to form inclusion bodies in a cell as compared to an onconase polypeptide having the amino acid sequence of SEQ ID NO: 1.

In some embodiments, the onconase polypeptide comprises the amino acid sequence of any one of SEQ ID NOs: 2-10 and 15-22 operably linked to one or more oligopeptides, optionally with one, two, three, four, five or all six C-terminal histidine residues deleted as shown in FIG. 20A. In some embodiments, the cell expresses a fusion polypeptide comprising an onconase polypeptide comprising an amino acid sequence that has at least 80%, at least 85%, at least 90%, at least 91%, at least 92%, at least 93%, at least 94%, at least 95%, at least 96%, at least 97%, at least 98%, or at least 99% sequence identity to any of SEQ ID NOs: 2-10 and 15-22, optionally with one, two, three, four, five or all six C-terminal histidine residues deleted as shown in FIG. 20A. In some embodiments, the onconase polypeptide comprises at least one amino acid sequence that has between 80% to 85%, 85% to 90%, 90% to 95%, or 95% to 100% sequence identity with any one of SEQ ID NOs: 1-10 and 15-22, optionally with one, two, three, four, five or all six C-terminal histidine residues deleted as shown in FIG. 20A. In some embodiments, the one or more oligopeptides are 4-50 or 5-30 amino acids long. In some embodiments, the operable linkage comprises an Asp-Pro bond.

In some embodiments, the onconase polypeptide with a modified N-terminus and/or C-terminus operably linked to one or more oligopeptides. In some embodiments, the cell expresses a fusion polypeptide comprising an onconase polypeptide one or more amino acid substitution in the N-terminus and/or C-terminus as compared to SEQ ID NO: 1. In some embodiments, the cell expresses a fusion polypeptide comprising an onconase polypeptide comprising 11 amino acid substitutions in the N-terminus and/or C-terminus as compared to SEQ ID NO: 1. In some embodiments, the cell expresses a fusion polypeptide comprising an onconase polypeptide 11 amino acid substitutions in the N-terminus as compared to SEQ ID NO: 1. In some embodiments, the one or more oligopeptides are 4-50 or 5-30 amino acids long. In some embodiments, the operable linkage comprises an Asp-Pro bond.

In some embodiments, the cell expresses a fusion polypeptide comprising an insoluble carrier polypeptide comprising a TAF12 polypeptide. In some embodiments, the TAF12 polypeptide comprises the amino acid sequence of SEQ ID NO: 23 operably linked to one or more oligopeptides by a peptide bond. In some embodiments, the TAF12 polypeptide comprises an amino acid sequence that has at least 80%, at least 85%, at least 90%, at least 91%, at least 92%, at least 93%, at least 94%, at least 95%, at least 96%, at least 97%, at least 98%, or at least 99% sequence identity to SEQ ID NO: 23. In some embodiments, the TAF12 polypeptide comprises an amino acid sequence that has between 80% to 85%, 85% to 90%, 90% to 95%, or 95% to 100% sequence identity to SEQ ID NO: 23. In some embodiments, the TAF12 polypeptide comprises one or more amino acid substitutions, or one or more insertions or deletions, as compared to the amino acid sequence of SEQ ID NO: 23. In some embodiments, the one or more amino acid substitutions, or one or more insertions or deletions, decrease susceptibility of the TAF12 to chemical cleavage as compared to a TAF12 polypeptide having the amino acid sequence of SEQ ID NO: 23. In some embodiments, the one or more amino acid substitutions, or one or more insertion or deletions results in a TAF12 polypeptide that has increase yield or higher capacity to form inclusion bodies in a cell as compared to a TAF12 polypeptide having the amino acid sequence of SEQ ID NO: 23. In some embodiments, the one or more oligopeptides are 4-50 or 5-30 amino acids long. In some embodiments, the operable linkage comprises an Asp-Pro bond.

In some embodiments, the TAF12 polypeptide with a modified N-terminus and/or C-terminus operably linked to one or more oligopeptides. In some embodiments, the cell expresses a fusion polypeptide comprising a TAF12 polypeptide one or more amino acid substitution in the N-terminus and/or C-terminus as compared to SEQ ID NO: 23. In some embodiments, the cell expresses a fusion polypeptide comprising a TAF12 polypeptide comprising 11 amino acid substitutions in the N-terminus and/or C-terminus as compared to SEQ ID NO: 23. In some embodiments, the cell expresses a fusion polypeptide comprising a TAF12 polypeptide 11 amino acid substitutions in the N-terminus as compared to SEQ ID NO: 23. In some embodiments, the one or more oligopeptides are 4-50 or 5-30 amino acids long. In some embodiments, the operable linkage comprises an Asp-Pro bond.

In some embodiments, the cell expresses an oligopeptide. In some embodiments, the oligopeptide is 4-50 or 5-30 amino acids long. In some embodiments, the cell expresses an oligopeptide comprising an active amino acid sequence. In some embodiments, the active amino acid sequence is a miPEP sequence. In some embodiments, the miPEP sequence regulates a miRNA. In some embodiments, the miPEP sequence regulates a plant miRNA. In some embodiments, the miPEP comprises an N-terminal proline, a C-terminal aspartic acid, or both the N-terminal proline and the C-terminal aspartic acid.

In some embodiments, the cell expresses an oligopeptide comprising the amino acid sequence of any one of SEQ ID NOs: 12-14 and 31-2401. In some embodiments, the oligopeptide comprises an amino acid sequence that has at least 80%, at least 85%, at least 90%, at least 91%, at least 92%, at least 93%, at least 94%, at least 95%, at least 96%, at least 97%, at least 98%, or at least 99% sequence identity with any one of SEQ ID NOs: 12-14 and 31-2401. In some embodiments, the oligopeptide comprises an amino acid sequence that has between 80% to 85%, 85% to 90%, 90% to 95%, or 95% to 100% sequence identity with any one of SEQ ID NOs: 12-14 and 31-2401. In some embodiments, the cell expresses an oligopeptide comprising at least one of the amino acid sequence of any one of SEQ ID NOs: 12-14 and 31-2401.

In some embodiments, the oligopeptide is 4-50 or 5-30 amino acids long. In some embodiments, oligopeptide comprises an active amino acid sequence and an N-terminal proline, a C-terminal aspartic acid, or both the N-terminal proline and the C-terminal aspartic acid.

In some embodiments, the cell expresses an onconase polypeptide. In some embodiments, the onconase polypeptide is a variant onconase polypeptide. In some embodiments, the onconase polypeptide comprises the amino acid sequence of SEQ ID NO: 1. In some embodiments, the onconase polypeptide comprises an amino acid sequence that has at least 80%, at least 85%, at least 90%, at least 91%, at least 92%, at least 93%, at least 94%, at least 95%, at least 96%, at least 97%, at least 98%, or at least 99% sequence identity to SEQ ID NO: 1. In some embodiments, the onconase polypeptide comprises an amino acid sequence that has between 80% to 85%, 85% to 90%, 90% to 95%, or 95% to 100% sequence identity to SEQ ID NO: 1. In some embodiments, the cell expresses an onconase polypeptide comprising one or more amino acid substitutions compared to the amino acid sequence of SEQ ID NO: 1. In some embodiments, the onconase polypeptide comprises one or more insertions or deletions as compared to the amino acid sequence of SEQ ID NO: 1. In some embodiments, the cell expresses an onconase polypeptide comprising one or more amino acid substitutions that decrease susceptibility to chemical cleavage as compared to an onconase polypeptide having the amino acid sequence of SEQ ID NO: 1. In some embodiments, the cell expresses an onconase polypeptide comprising one or more deletions or insertions that reduce susceptibility to chemical cleavage as compared to an onconase polypeptide having the amino acid sequence of SEQ ID NO: 1.

In some embodiments, the cell expresses an onconase polypeptide comprising one or more amino acid substitutions, or one or more insertions or deletions that increase yield or promote formation of inclusion bodies as compared to an onconase polypeptide having the amino acid sequence of SEQ ID NO: 11.

In some embodiments, the cell expresses an onconase polypeptide comprising the amino acid sequence of any one of SEQ ID NOs: 2-10 and 15-22, optionally with one, two, three, four, five or all six C-terminal histidine residues deleted as shown in FIG. 20A. In some embodiments, the onconase polypeptide comprises an amino acid sequence that has at least 80%, at least 85%, at least 90%, at least 91%, at least 92%, at least 93%, at least 94%, at least 95%, at least 96%, at least 97%, at least 98%, or at least 99% sequence identity to any of SEQ ID NOs: 2-10 and 15-22, optionally with one, two, three, four, five or all six C-terminal histidine residues deleted as shown in FIG. 20A. In some embodiments, the onconase polypeptide comprises at least one amino acid sequence that has between 80% to 85%, 85% to 90%, 90% to 95%, or 95% to 100% sequence identity with any one of SEQ ID NOs: 12-14 and 31-2401. In some embodiments, the onconase polypeptide comprises a modified N-terminus and/or C-terminus. In some embodiments, the onconase polypeptide comprises one or more amino acid substitution in the N-terminus and/or C-terminus as compared to SEQ ID NO: 1. In some embodiments, the onconase polypeptide comprises 11 amino acid substitutions in the N-terminus as compared to SEQ ID NO: 1. In some embodiments, the onconase polypeptide comprises a sequence tag.

In some embodiments, the cell expresses a TAF12 polypeptide. In some embodiments, the TAF12 polypeptide is a variant TAF12 polypeptide. In some embodiments, the TAF12 polypeptide comprises the amino acid sequence of SEQ ID NO: 23. In some embodiments, the TAF12 polypeptide comprises an amino acid sequence that has at least 80%, at least 85%, at least 90%, at least 91%, at least 92%, at least 93%, at least 94%, at least 95%, at least 96%, at least 97%, at least 98%, or at least 99% sequence identity to SEQ ID NO: 23. In some embodiments, the TAF12 polypeptide comprises an amino acid sequence that has between 80% to 85%, 85% to 90%, 90% to 95%, or 95% to 100% sequence identity to SEQ ID NO: 23. In some embodiments, the cell expresses a TAF12 polypeptide comprising one or more amino acid substitutions compared to the amino acid sequence of SEQ ID NO: 23. In some embodiments, the TAF12 polypeptide comprises one or more insertions or deletions as compared to the amino acid sequence of SEQ ID NO: 23. In some embodiments, the cell expresses a TAF12 polypeptide comprising one or more amino acid substitutions that decrease susceptibility to chemical cleavage as compared to a TAF12 polypeptide having the amino acid sequence of SEQ ID NO: 23. In some embodiments, the cell expresses a TAF12 polypeptide comprising one or more deletions or insertions that reduce susceptibility to chemical cleavage as compared to a TAF12 polypeptide having the amino acid sequence of SEQ ID NO: 23.

In some embodiments, the cell expresses a TAF12 polypeptide comprising one or more amino acid substitutions, or one or more insertions or deletions that increase yield or promote formation of inclusion bodies as compared to a TAF12 polypeptide having the amino acid sequence of SEQ ID NO: 11.

In some embodiments, the TAF12 polypeptide comprises at least one amino acid sequence that has between 80% to 85%, 85% to 90%, 90% to 95%, or 95% to 100% sequence identity with any one of SEQ ID NOs: 12-14 and 31-2401. In some embodiments, the TAF12 polypeptide comprises a modified N-terminus and/or C-terminus. In some embodiments, the TAF12 polypeptide comprises one or more amino acid substitution in the N-terminus and/or C-terminus as compared to SEQ ID NO: 23. In some embodiments, the TAF12 polypeptide comprises 11 amino acid substitutions in the N-terminus as compared to SEQ ID NO: 23. In some embodiments, the TAF12 polypeptide comprises a sequence tag.

### X. Kits

Some aspects of the present disclosure provide for kits comprising any of the fusion polypeptides, insoluble carrier polypeptides (e.g., onconase polypeptides or TAF12 polypeptides), or oligopeptides of the disclosure; any of the nucleic acids or vectors encoding the fusion polypeptides, insoluble carrier polypeptides (e.g., onconase polypeptides or TAF12 polypeptides), or oligopeptides of the disclosure; or any of the cells expressing the fusion polypeptides, insoluble carrier polypeptides (e.g., onconase polypeptides or TAF12 polypeptides), or oligopeptides of the disclosure.

Also provided herein are kits for use with any of the methods of the disclosure.

### EXAMPLES

The following examples further illustrate the invention but should not be construed as in any way limiting its scope. In light of the present disclosure and the general level of skill in the art, those of skill will appreciate that the following Examples are intended to be exemplary only and that numerous changes, modifications, and alterations can be employed without departing from the scope of the presently disclosed subject matter. The attached figures are meant to be considered as integral parts of the specification and description of the disclosure.

### Example 1: Conditions for Release of Peptides from Onconase Inclusion Bodies

This example describes the evaluation of conditions for cleavage of D-P bonds to release a peptide from an onconase inclusion body for production of short peptides in E. coli.

### Methods

Peptide Release from an Onconase

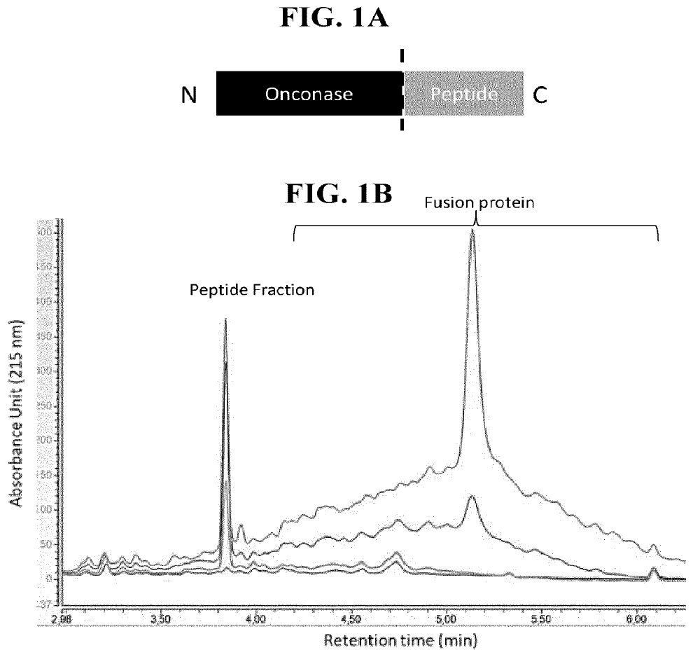

The onconase fusion construct of (FIG. 1A) was expressed in E. coli. The bacteria cells were pelleted, after which the cell pellet was lysed through sonication. The cell lysate was pelleted by centrifugation. The pellet was then washed 3× times with TritonX-100 buffer (0.1 M Tris, 2% TritonX-100, 2 M urea, 10 mM EDTA, pH 7.4), followed by 2× washes with wash buffer (1 M Tris, pH 7.4), then washed with water. The purified inclusion bodies were then subject to chemical cleavage by adjusting to the target pH with HCl and incubation at 80° C. for 16 hours.

### Analysis of Peptide Expression

Reversed-phase-high performance liquid chromatography (RP-HPLC) analysis was used to estimate peptide yield and solubilization of the onconase (FIG. 1B).

### Results

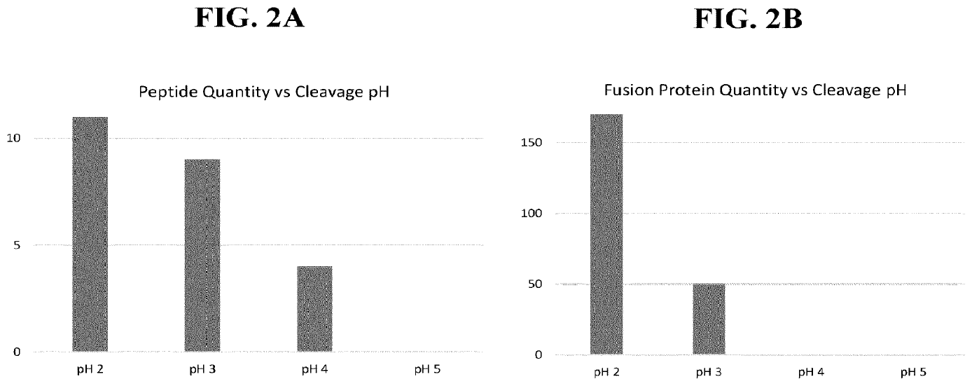

In the first experiment, four pHs between 2 and 5 were tested (FIGS. 2A-2B). The onconase remained mostly as inclusion bodies, but the chemical cleavage was still reasonably efficient. The cleavage efficiency was most efficient in the range of pH 2 and pH 3.

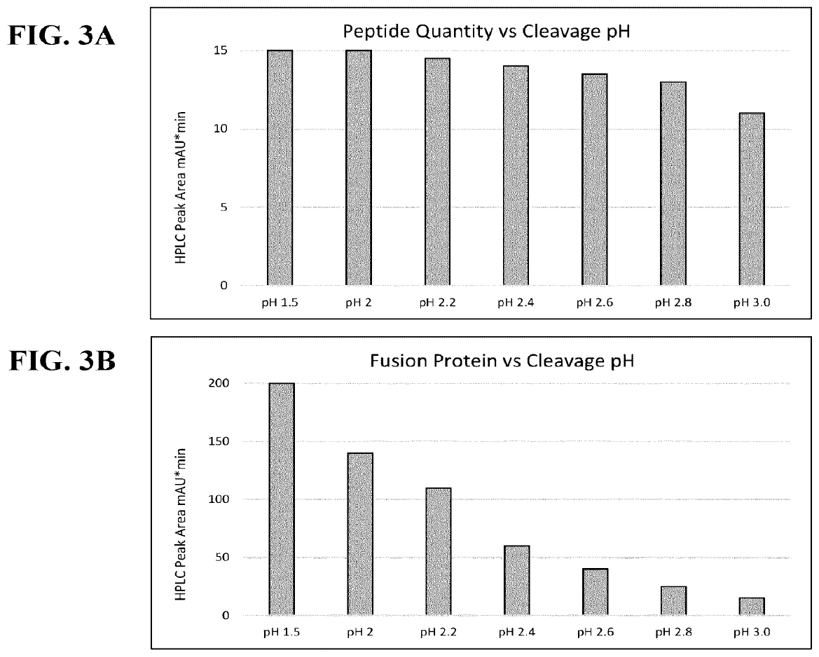

A second experiment was conducted to determine an optimal balance between an efficient pH cleavage with a minimal amount of solubilized fusion protein. A cleavage pH from 2.4 to 2.8 were the optimal cleavage condition with a high level of released peptide and a low amount of solubilized fusion proteins (FIGS. 3A-B). Similar results were obtained using sulfuric acid to adjust the pH (data not shown).

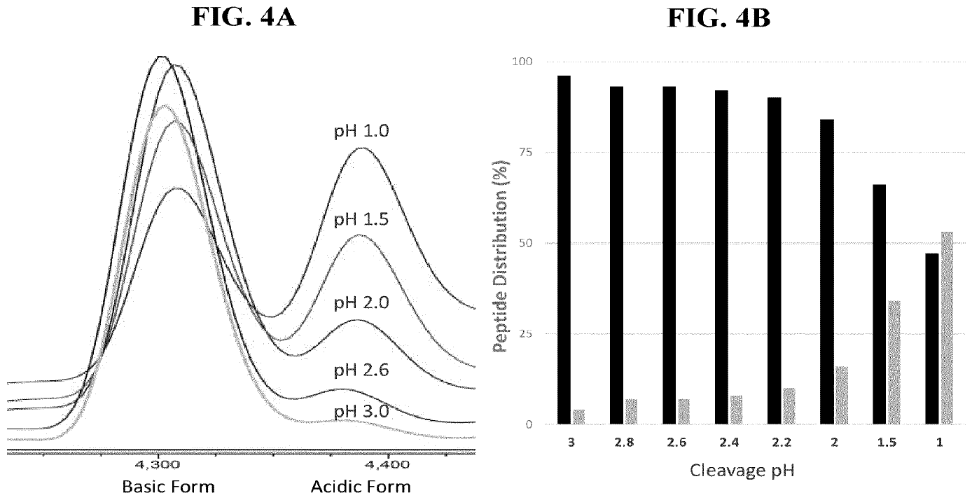

To assess impact on amine groups, a model peptide containing a glutamine residue was tested. As depicted in FIGS. 4A-B, when cleavage pH is lowered below pH 3.0, glutamine containing peptide began to be converted from a basic form into an acidic form, which could be separated by reverse phase HPLC (FIG. 4A). This indicated that the glutamine was converted into glutamic acid under acidic conditions, likely through a deamidation reaction which is catalyzed by high temperature and acid conditions. This observation emphasizes the importance to work at pH above 2.6 for pH sensitive peptides bearing either asparagine or glutamine.

### Example 2: Comparison of Different Inclusion Body Forming Proteins

This example describes the evaluation of different inclusion body forming proteins for production of short peptides in E. coli.

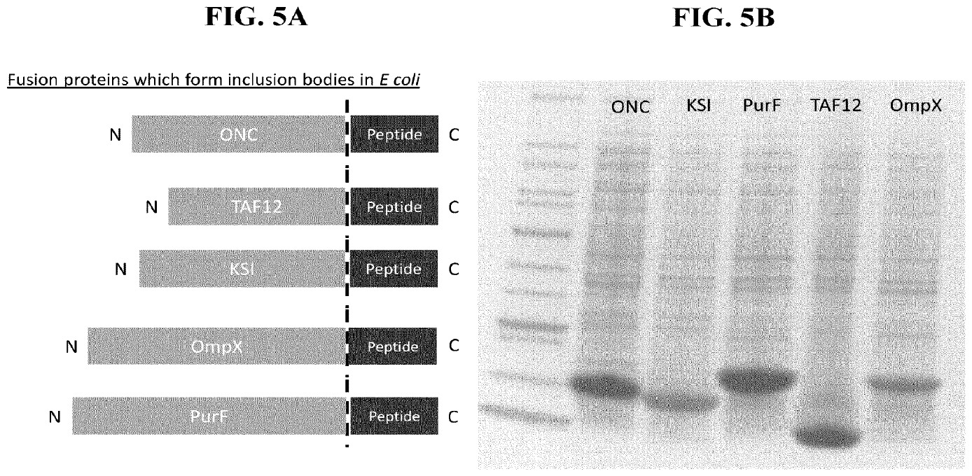

Five different inclusion body forming proteins were compared (FIG. 5A): delta(5)-3-Ketosteroid isomerase (KSI), a polypeptide F4 fragment derived from PurF protein of 16.7 kDa (PurF); OmpX of E. coli (OmpX), the histone fold domain of the human transcription factor TAF12 (TAF12), and onconase. All constructs were cloned into pET28a plasmid-based expression system and BL21(DE3) bacterial strains were transformed and selected on kanamycin (50 μg/ml). Expressions tests were conducted 3 times independently in auto-inducible medium at 37° C. in flask (FIG. 5B). Peptide yields of the test Peptide A were estimated after chemical cleavage at pH 2.6 at 80° C. (overnight incubation ~16 hours) by RP-HPLC C18 column using a synthetic peptide of the same amino acid sequence at known concentrations.

All 5 fusions proteins were efficiently expressed and the bioproduced peptides were released under the test conditions.

### Example 3: A Concatemeric Strategy for Peptide Production in E. coli

This example describes the evaluation of a concatemeric strategy for production of short peptides in E. coli.

### Methods

### Peptide Production Using Onconase Concatemer

The onconase fusion constructs were expressed in E. coli. The bacteria cells were pelleted, after which the cell pellet was lysed through sonication. The cell lysate was pelleted by centrifugation. The pellet was then washed 3× times with TritonX-100 buffer (0.1 M Tris, 2% TritonX-100, 2 M urea, 10 mM EDTA, pH 7.4), followed by 2× washes with wash buffer (1 M Tris, pH 7.4), then, washed with water. The purified inclusion bodies were then solubilized by incubation in solubilization buffer (6 M guanidinium chloride, Tris 50 mM, 10 mM beta-mercaptoethanol, pH 7.4). The solubilized proteins were then refolded for onconase precipitation by extensive dialysis at 4° C. in 0.1 M acetic acid at pH 3.0, which results in guanidinium hydrochloride removal. The soluble fraction was cleaved by incubation in 0.1 M acetic acid at pH 2 and 60° C. for 24 hours. After cleavage, the onconase was precipitated from the solution by addition of NH₄OH buffer (pH 7.0-7.2). The peptide was then lyophilized for ammonium acetate removal and subsequently centrifuged.

### Analysis of Peptide Expression

Expression of the proteins was assessed by SDS-PAGE analysis of the insoluble fraction. Expression tests were performed at 37° C. in a commercial auto-inducible medium (Formedium, Ref. AIMTB02) using the BL21(DE3)STAR bacterial strain (Invitrogen, Ref. C601003).

### Results

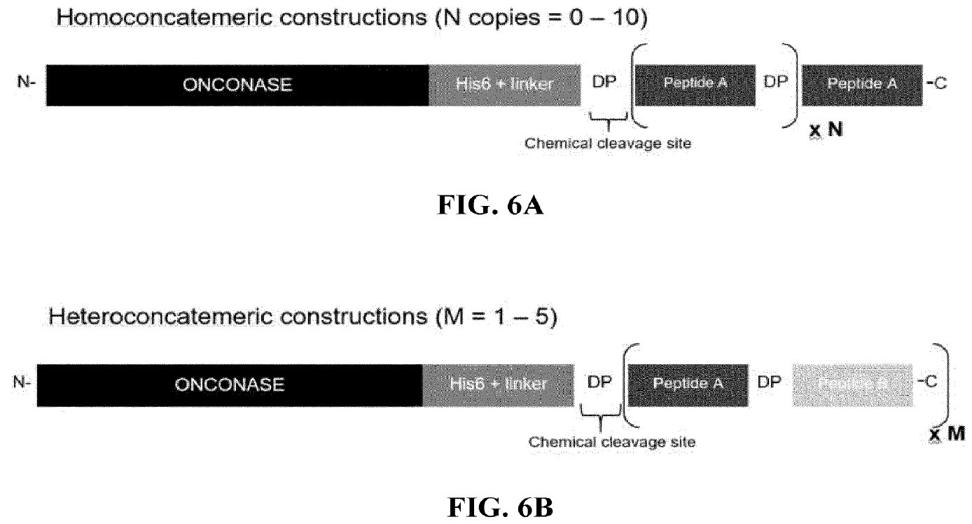

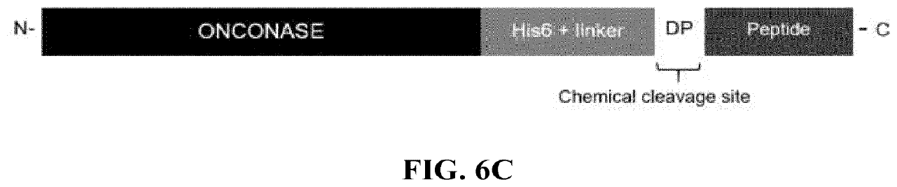

A concatemeric onconase fusion strategy could be used to increased yield of small peptides (e.g. 10 amino acid long peptides). Concatemeric constructs can be designed as either homoconcatemers (FIG. 6A), with several copies of the same peptide separated by an acid cleavage site fused to the C-terminus of the onconase, or as heteroconcatemers (FIG. 6B), with different peptide sequences being added.

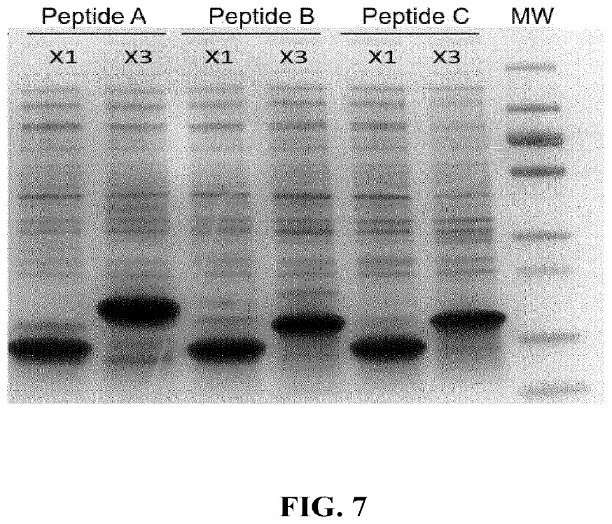

To determine whether the concatemeric strategy could be applied for efficient peptide production, onconase fusions with three different 10 amino acid-long hydrophilic peptides were prepared. The onconase fusions had either one or three copies of each peptide. As shown in FIG. 7, both the homoconcatemeric constructs and the heteroconcatemeric constructs were expressed at high levels. The addition of up to three copies of each peptide did not affect protein accumulation in inclusion bodies in large quantities.

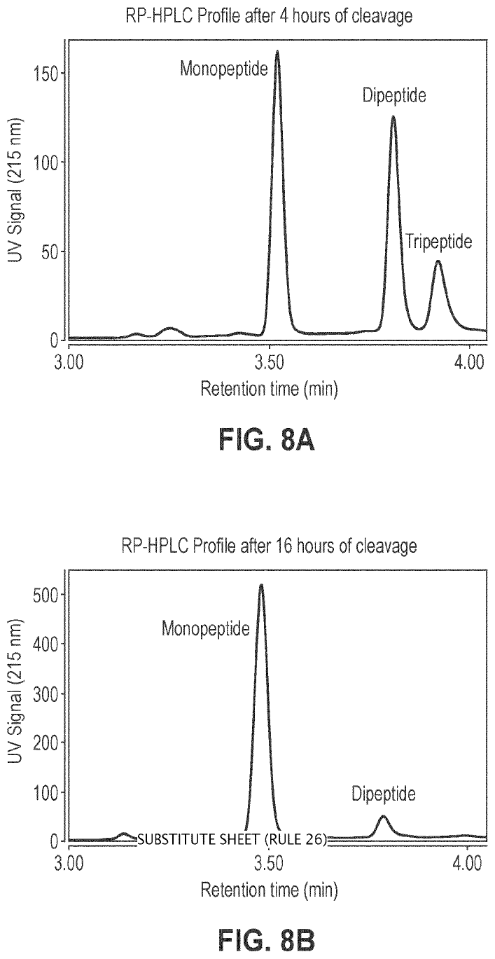

To confirm that the concatemeric strategy is applicable to other inclusion body forming proteins, a fusion TAF12 construct with three identical peptides fused to the C-terminus was expressed. The inclusion bodies were washed as in Example 3 and then subject to cleavage by adjustment of the pH with HCl to pH 2.6 and incubation at 80° C. As shown on FIG. 8A, after 4 hours of cleavage, intermediate species corresponding to mono di- and tri-peptide species could be detected. With extended cleavage time, almost everything has been converted into the single peptide specie of interest (FIG. 8B).

### Example 4: Onconase N-Terminus Modifications for Increased Peptide Yield

This example describes the evaluation of variant onconase proteins for increasing peptide yield and purity.

### Methods

### Peptide Production Using Onconase Concatemers

Peptide production was performed as described in Example 3.

### Analysis of Peptide Yield and Quality

Reversed-phase-high performance liquid chromatography (RP-HPLC) analysis was used to estimate peptide yield. An average of three independent purifications were performed using an auto-inducible medium on a 50-ml flask scale.

The peptide quality analysis was performed by RP-HPLC with a C18 column (waters). The main miPEP species was quantified by comparison to injection of a known quantity of a chemically synthetized peptide (sb-PEPTIDE), which eluted at the same retention time. The yield per L of culture volume was extrapolated from the average signal detected, with three independents purifications for each construct.

### Results

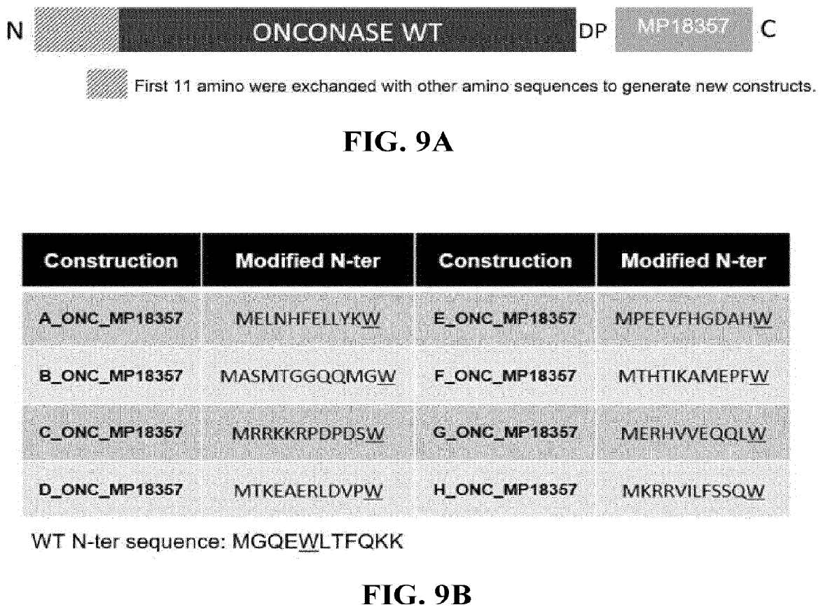

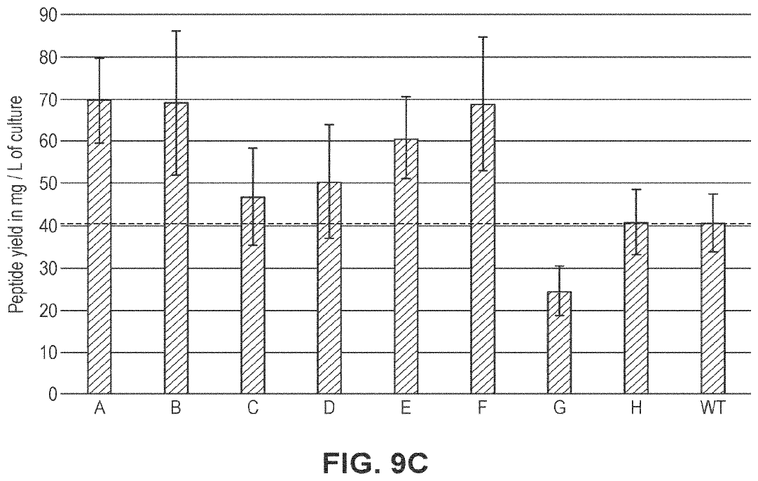

Eight new onconase constructs with variant N-terminus sequences were generated (FIGS. 9A-9B), and the yield for the MP18357 peptide (SEQ ID NO: 12) using each construct was compared then compared. Each onconase fusion construct tested had only one copy of MP18357 at the C-terminus (FIG. 9A). The peptide quality and quantity were determined by reverse-phase-high performance liquid chromatography (RP-HPLC).

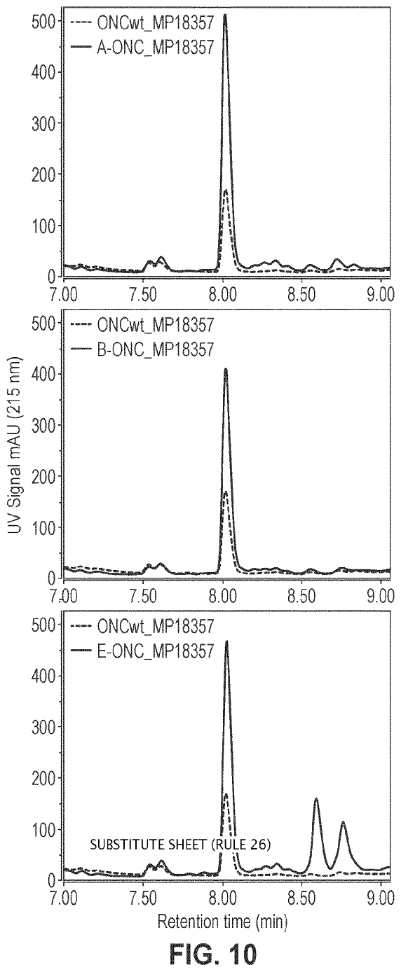

In contrast to the original onconase sequence, peptide yield increased by over 70% with 3 of the 8 constructs tested (FIG. 9C). Constructs A and B (FIG. 9B) yielded higher quantity of peptide, with similar peptide quality observed with the variant onconase as compared to the original onconase sequence (FIG. 10). Using constructs A and B, the main peptide species eluted at the same elution volume without detection of other important species (FIG. 10, insert in top and middle panels). Construct E represented an unsuccessful N-terminal modification, which resulted in higher quantities of peptide but of lesser quality (FIG. 10, bottom panel). The additional peptide species observed with may be due to non-specific cleavage.

### Example 5: Chemical Cleavage to Increase Yield and Peptide Homogeneity of Concatamerically-Produced Peptides

### Methods

Peptide purification and analysis of peptide yield was performed as described in Examples 3 and 4, with some modifications. Peptide cleavage was performed by solubilization and incubation of the soluble fraction in 20% acetic acid at pH 2 for 24 hours at either 40° C., 60° C., or 80° C. Peptide yield and quality was assessed by RP-HPLC at 2, 4 or 20 hours from start of incubation.

### Results

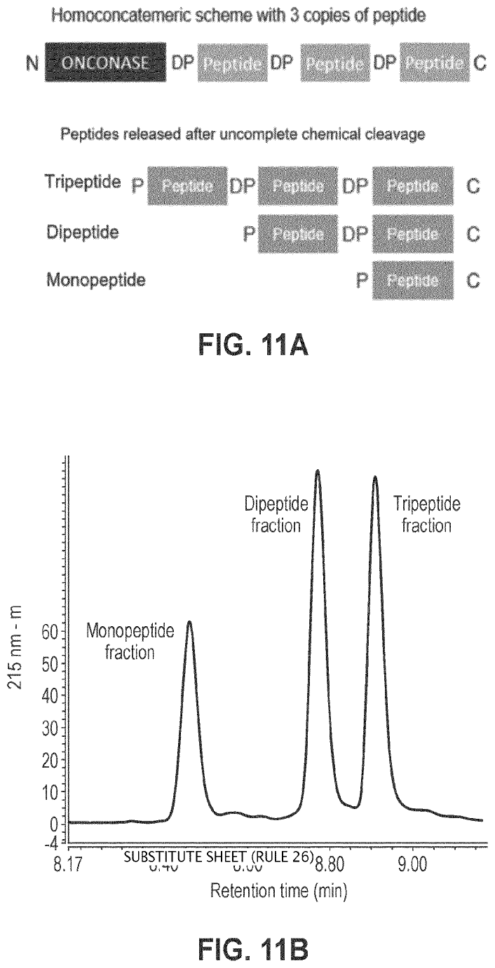

The use of homoconcatemer onconase fusions carrying three peptide copies resulted in incomplete peptide cleavage when performing acidic cleavage at 60° C. in acetic acid (FIGS. 11A-11B). Additional peaks were detected by RP-HPLC, indicating the presence of uncleaved dipeptide or tripeptide species (FIGS. 11A-11B).

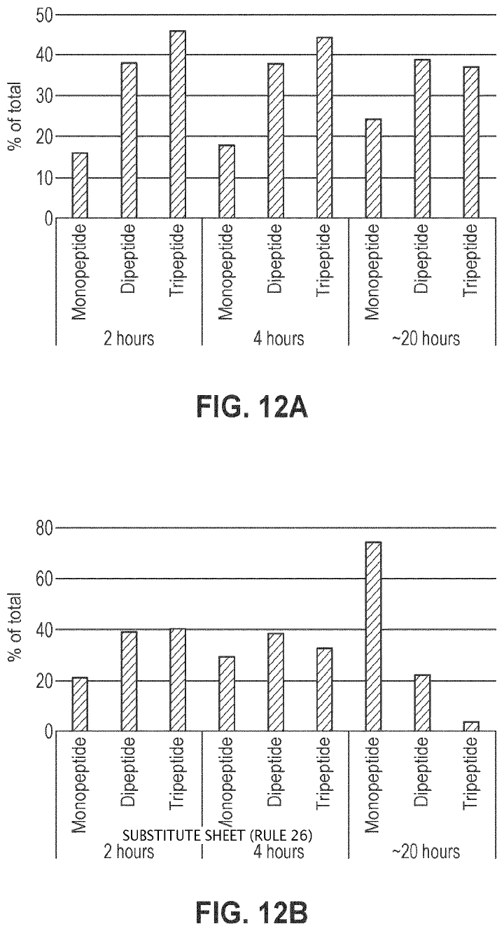

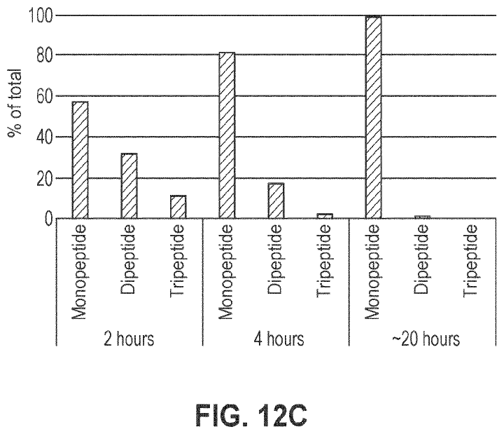

To improve cleavage efficiency for release to be closer to 100% cleavage, different incubation times and cleavage temperatures were tested. As shown in FIG. 12A, at 40° C., incubation time marginally impacted cleavage efficiency, with around 25% of the peptide found as monopeptides even after 20 hours. Increasing the temperature to 60° C. was found to lead to better cleavage than 40° C., resulting in 75% of the peptide produced as monopeptide species (FIG. 12B). At 80° C., cleavage was better after 4 hours than at 60° C., with complete cleavage of the peptides was observed after 20 hours (FIG. 12C), and only a monopeptide fraction being recovered. The peptide quality was not affected by the use of a concatemeric construct. No further improvements were observed upon changing of the acetic acid concentration within a 20-50% range.

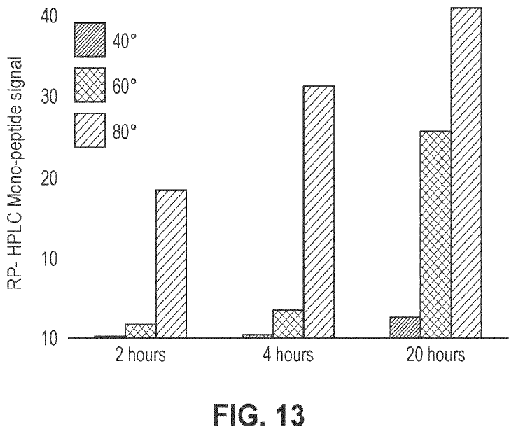

Increasing the cleavage temperature improved both the peptide homogeneity (FIGS. 12A-12C) and the peptide yield (FIG. 13) when using a concatemeric strategy as well as reducing the peptide purification time. The peptide yield at 80° C. was around 60% higher than at 60° C., mostly resulting from a higher monopeptide fraction recovery.

### Example 6: Direct Acetic Acid Solubilization and Development of a Chaotropic-Free Protocol

This example describes the development of a chaotropic-free protocol for peptide production using an onconase fusion construct.

### Methods

Peptide purification and analysis of peptide yield was performed as described in Examples 3 and 4, with some modifications. Direct solubilization of the inclusion bodies was performed using a protein concentration of either 20 g/L or 50 g/L incubated in formic acid or acetic acid at 60° C. or 80° C. Incubation was performed with 20%, 30%, 40% or 50% of acetic acid or formic acid (w/v).

### Results

Onconase solubility is highly pH-dependent and the onconase-fusion peptide production strategy is based on this feature. Direct solubilization at acidic pH might be sufficient to solubilize onconase and directly trigger acidic chemical cleavage at 80° C.

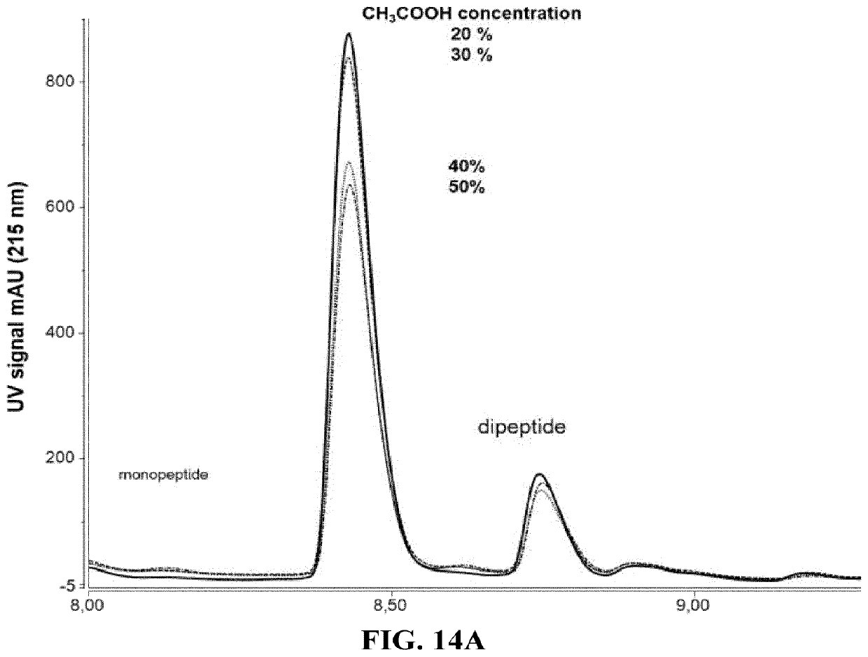

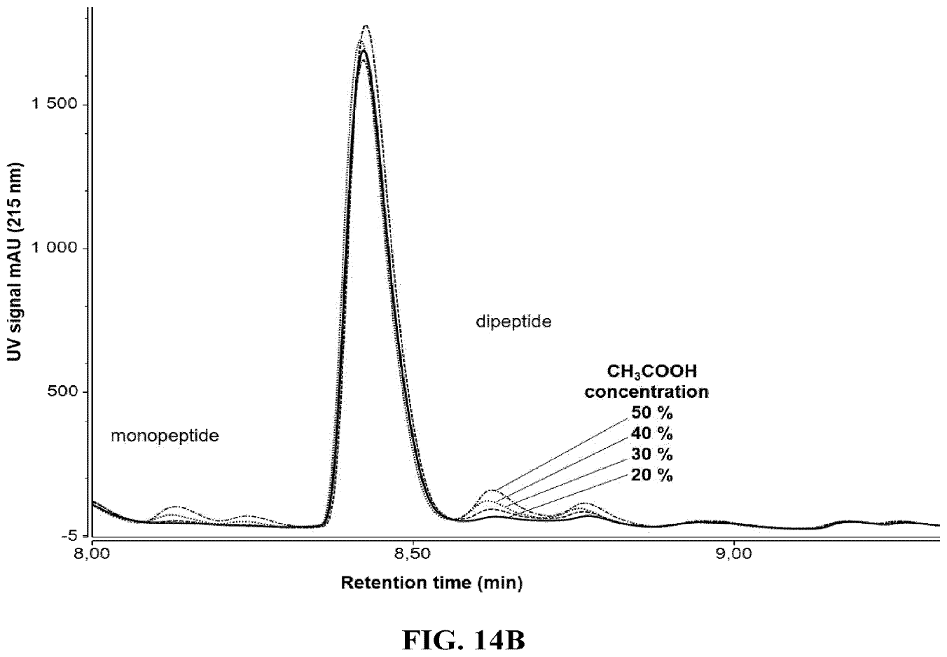

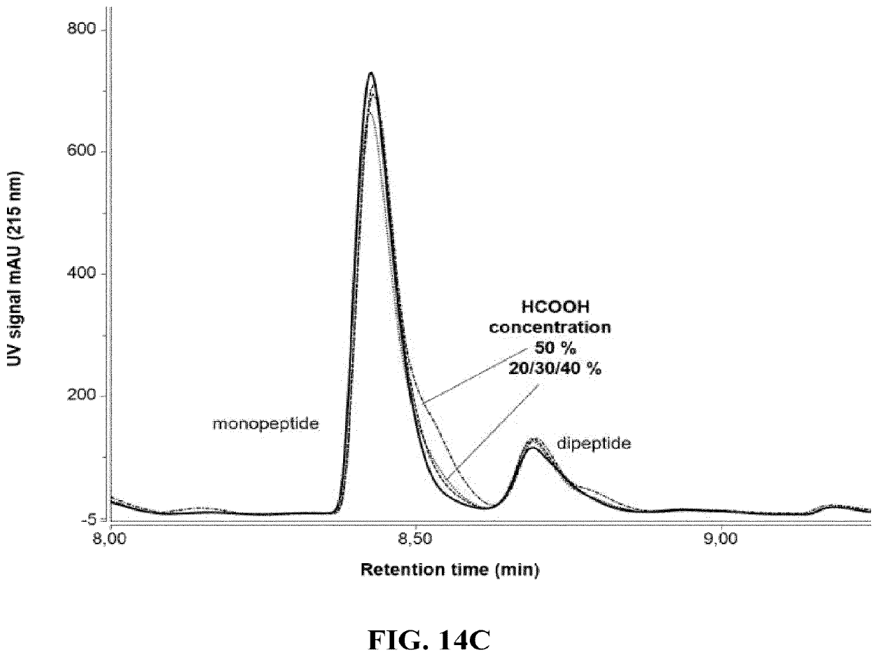

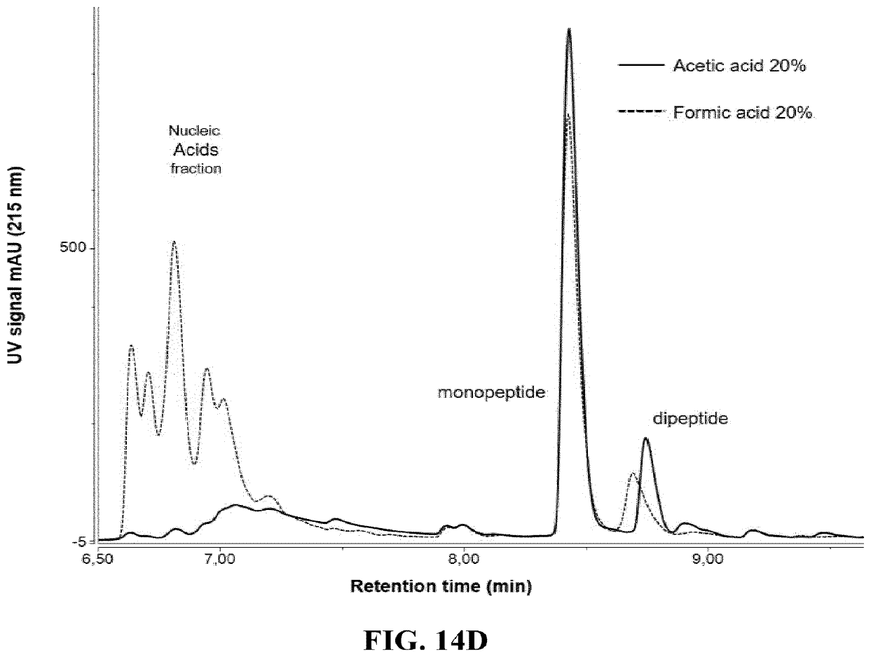

To test whether acidic pH conditions might sufficiently stabilize onconase and trigger acid cleavage, different concentrations of formic acid or acetic acid were tested for the ability to visually solubilize onconase inclusion bodies and directly perform peptide cleavage. Two concentrations of inclusion bodies (20 g/L at 60° C. and 50 g/L at 80° C.) were tested with either formic acid or acetic acid at concentrations ranging from 20-50% (FIGS. 14A-14B). Direct solubilization and cleavage of onconase was observed at 80° C. even when using an acetic acid concentration as low as 20% (w/v) and a high protein concentration of 50 g/L. Two peptide species were detected when the reaction was performed at 60° C., which corresponded to monopeptide and dipeptide species (FIG. 14A). The acetic concentration did not affect the peptide yield or quality. Increasing acetic acid concentration appeared to be detrimental, as a slight increase of dipeptide content was observed when acetic acid concentration was increased (FIG. 14B). FIGS. 14C and 14D show that cleavage with formic acid worked as well as acetic acid, but chemical cleavage was less specific than with acetic acid. As shown in FIG. 14C, with higher formic acid a shoulder was observed, suggesting a negative effect. The use of lower acetic acid concentrations further improves direct solubilization and cleavage while maximizing the protein concentration.

### Example 7: Development of Column-Free Downstream Purification Steps

In this example, the column-free downstream purification steps for peptide production using a concatemeric strategy were optimized.

### Methods

The onconase fusion constructs were expressed in bacteria cells. The bacteria cells were pelleted, after which the cell pellet was lysed through sonication. The cell lysate was pelleted by centrifugation. The pellet was then washed 2 times with TritonX-100 buffer at 37° C. (0.05 M Tris, 2% TritonX-100, pH 7.5), followed by 2× washes with high salt buffer (1 M NaCl, pH 12) and a 5-minute heat shock at 95° C., and finally washed 2× times with water. Inclusion bodies were then recovered by centrifugation after cooling down. The purified inclusion bodies were then solubilized and cleaved in acetic acid. After cleavage, the onconase was precipitated from the solution by addition of NH₄OH to pH 7.0-7.2. The peptide was then lyophilized for ammonium acetate removal and subsequently centrifuged.

### Results

Many current methods for peptide production require column-based separation methods. However, these methods result in additional steps and add cost to peptide production. Thus, production of peptides with a column-free method presents a significant advantage.

To optimize a column-free protocol, alternative steps for inclusion body isolation were tested. For example, solubilization with 2% SDS was effective for solubilizing the inclusion bodies but inhibited downstream cleavage and peptide release. Sarkozyl was effective at solubilizing inclusion bodies but was not effective under acidic conditions, so other conditions were tested.

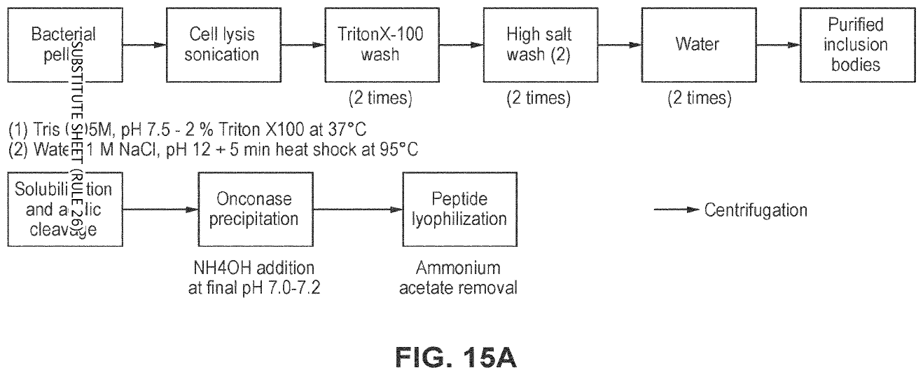

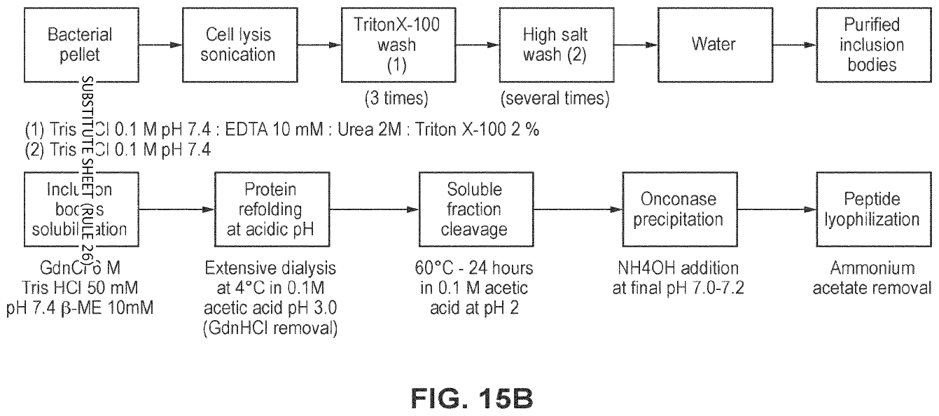

Washing inclusion bodies with a high concentration of salt (1 M NaCl) at basic pH, followed by a heat shock at 95° C. to induce DNA unfolding was found to be the most efficient protocol (FIG. 15A).

At pH above protein isoelectric point, inclusion bodies are negatively charged, favoring DNA repulsion after thermal denaturation. The high salt concentration helps to “shield” inclusion bodies and avoid attracting nucleic acids once the sample temperature is lowered. Salt, heat shock or basic pH alone was not as effective to remove nucleic acid and the combination of all those parameters are important in reducing nucleic acid contamination during peptide purification.

### Example 8: Assessment of Peptide Yield with Optimized Peptide Production and Purification Protocol

This example shows that direct solubilization of inclusion bodies resulted in higher yield of peptide product.

### Methods

### Peptide Purification Using a Chaotropic Agent

The onconase fusion constructs were expressed in bacteria cells. The bacteria cells were pelleted, after which the cell pellet was lysed through sonication. The cell lysate was pelleted by centrifugation. The pellet was then washed 3× times with TritonX-100 buffer (0.1 M Tris, 2% TritonX-100, 2 M urea, 10 mM EDTA, pH 7.4), followed by 2× washes with wash buffer (1 M Tris, pH 7.4), then, washed with water. The purified inclusion bodies were then solubilized by incubation in solubilization buffer (6 M guanidinium chloride, Tris 50 mM, 10 mM beta-mercaptoethanol, pH 7.4). The solubilized proteins were then refolded by extensive dialysis at 4° C. in 0.1 M acetic acid at pH 3.0, which results in guanidinium hydrochloride removal. The soluble fraction was cleaved by incubation in 0.1 M acetic acid at pH 2 and 60° C. for 24 hours. After cleavage, the onconase was precipitated from the solution by addition of NH₄OH to pH 7.0-7.2. After centrifugation at 10,000×g for 5 minutes, the aggregated fusion protein was in the pellet and the cleaved peptide is in the supernatant. The peptide was then lyophilized for ammonium acetate removal.

### Chaotropic Agent-Free Peptide Purification Protocol

The onconase fusion was expressed in bacteria cells. The bacteria cells were pelleted, after which the cell pellet was lysed through sonication. The cell lysate was pelleted by centrifugation. The pellet was then washed 2 times with TritonX-100 buffer at 37° C. (0.05 M Tris, 2% TritonX-100, pH 7.5), followed by 2× washes with high salt buffer (1 M NaCl, pH 12) and a 5-minute heat shock at 95° C., and finally washed 2× times with water. The purified inclusion bodies were then solubilized and cleaved in acetic acid. After cleavage, the onconase was precipitated from the solution by addition of NH₄OH buffer to pH 7.0-7.2. After centrifugation at 10,000×g for 5 minutes, the aggregated fusion protein was in the pellet and the cleaved peptide is in the supernatant. The peptide was then lyophilized for ammonium acetate removal.

### Results

To assess whether a chaotropic-free protocol could result in better yields than a protocol requiring the use of a chaotropic agent, the yield of peptide produced using each protocol was assessed and compared.

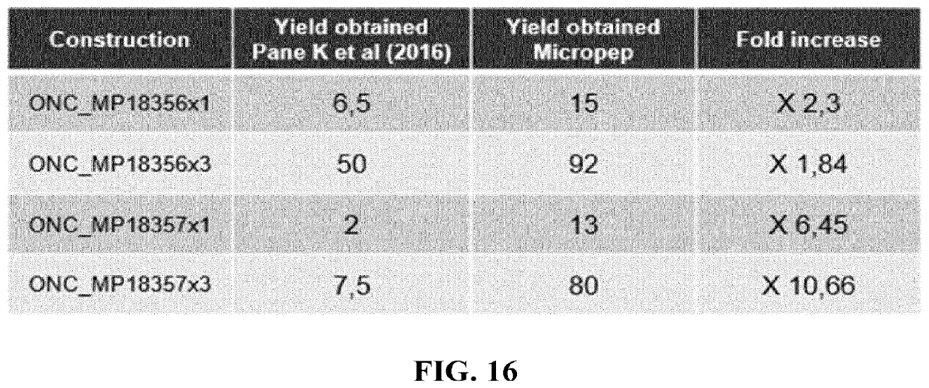

To this end, onconase fusions with either one or three (concatemeric) peptide copies were used for peptide production following either the standard protocol (FIG. 15B) or a chaotropic agent-free protocol (FIG. 15A). The chaotropic agent-free protocol (FIG. 15B), which substituted the use of chaotropic agent and directly solubilized the inclusion bodies in 20% acetic acid, resulted in higher yield for all evaluated constructs (FIG. 16). Moreover, increasing the solubilization and cleavage temperature to 80° C., resulted in high peptide homogeneity and in more than 80% pure peptide of interest in the absence of any column use. As the chaotropic agent-free protocol involves fewer steps, it enabled faster and less labor-demanding peptide production, with reduced buffer/chemical consumption.

### Example 9: Functional Assay Against Tomato Grey Mold (Botrytis cinerea)

In this example, the effect of the cleavage scar on peptide function is evaluated.

### Methods

### Peptide Production

Peptide production was performed using the chaotropic agent-free protocol described in Example 8.

A synthetic peptide (MP18913), which mimics the concatemeric sequence with an N-terminus proline (P) and C-terminus aspartate (D), was chemically synthetized and used as a control treatment.

### Disease Control Assay

Peptide activity was assessed by a disease control assay using tomato plants infected with grey mold (Botrytis cinerea). Peptides were sprayed 24 h post infection at concentration of 0.1 g/l or 0.3 g/l. Disease control percentage was determined based on convex hull recovery, with a total of 7 plants examined (n=7) upon infection with Botrytis spores at 250 spores/ml.

### Results

Acetic acid cleaves at aspartate-proline (Asp-Pro) peptide bonds. Chemical cleavage of the onconase concatemeric fusions using acetic acid thus results in peptides having an N-terminal Pro residue and a C-terminal Asp residue in addition to the peptide sequence.

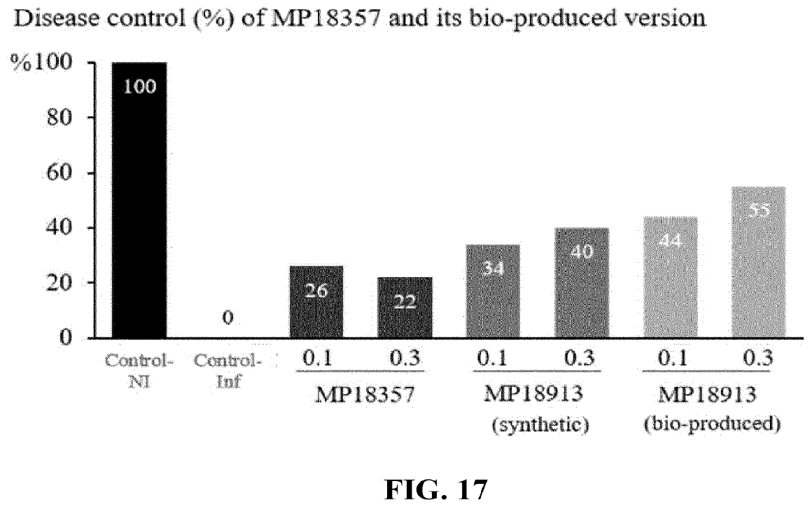

To test whether a peptide having the N-terminal Pro and C-terminal Asp amino acids was still functional, the MP18913 (SEQ ID NO: 14) peptide obtained either by bioproduction or from chemical synthesis were compared. MP18913 corresponds to a modified MP18357 peptide that has an additional proline residue (Pro) at the N-terminus and an aspartate residue (Asp) at C-terminus. Chemically-synthesized MP18913 mimics the concatemeric sequence with the addition of a proline (P) and an aspartate (D) at the N-terminus and C-terminus of the sequence, respectively. MP18913 was found to be as effective, or slightly more effective, than the chemically synthetized version in controlling tomato grey mold (Botrytis cinerea) infection (FIG. 17). Bio-produced MP18913 also provided even better protection against tomato grey mold than MP18357. The bio-produced version of MP18913 (MP18913-BPD) provides slightly better disease control, with up to 50% disease control, and appears to work best at 0.3 g/l. The addition of the proline and aspartate residues did not affect functionality of the peptides.

### Example 10: Evaluation of Concatemeric Constructs with Both N- and C-Peptide Additions

This example describes the generation of alternative concatemeric constructs for increased peptide yield.

### Methods

### Peptide Production

Peptide production is performed using the chaotropic agent-free protocol described in Example 8.

### Results

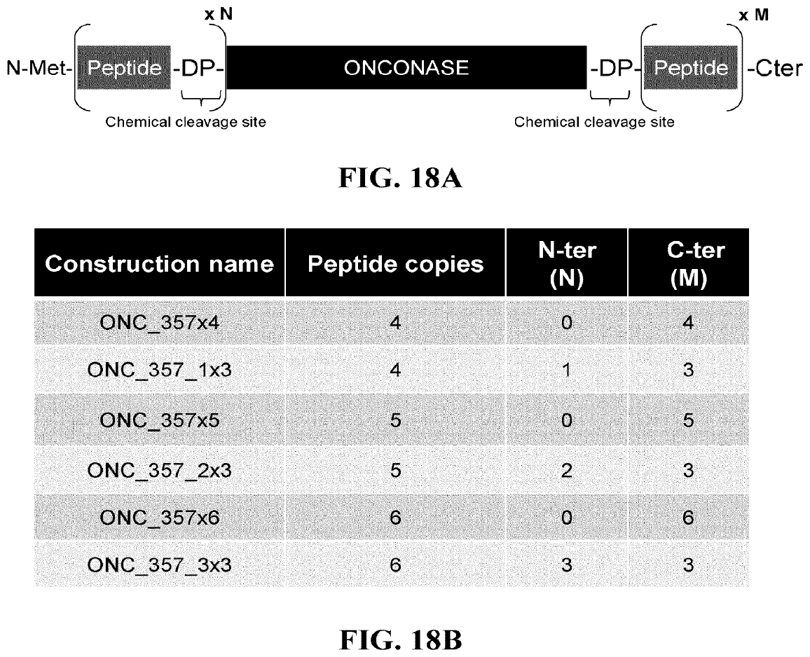

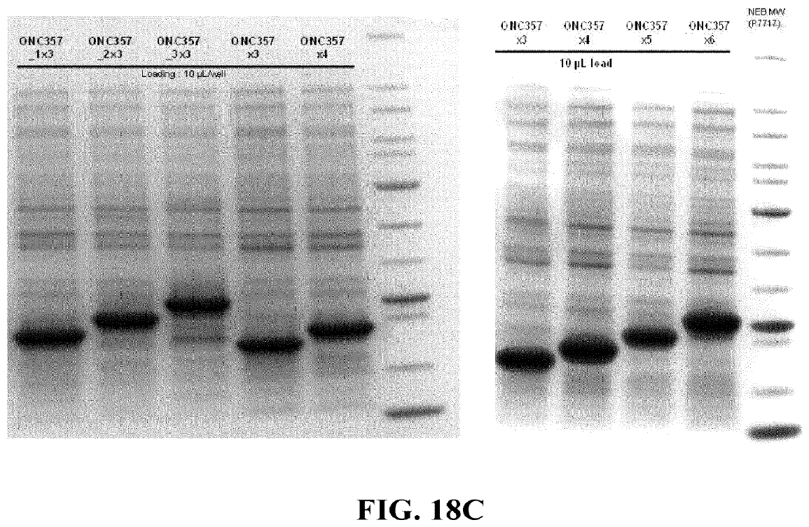

As described in Example 4, N-terminus modification of the onconase fusions is feasible and can lead to higher peptide yield. Concatemeric constructs with peptide copies at the N-terminus and/or additional peptide copies at the C-terminus (FIG. 18A) might also lead to increased peptide production.

To test whether onconase concatemers having N-terminal peptides could be used for increased peptide production, constructs expressing 4 to 6 copies of the MP18357 peptide were designed (FIG. 18A). These constructs are then expressed in E. coli and evaluated for expression and peptide yield. Using the same expression and purification method, the expression and peptide yield obtained using these constructs are compared to a similar construct having only peptide copy at the C-terminus.

### Results

As shown in FIG. 18B and FIG. 18C, each onconase fusion protein construct with up to six copies of the oligopeptide was expressed at a high level, regardless of whether the peptide was located at the N terminus or C terminus. This demonstrates that even up to six copies of the hydrophilic oligopeptide did not affect the onconase carrier activity.

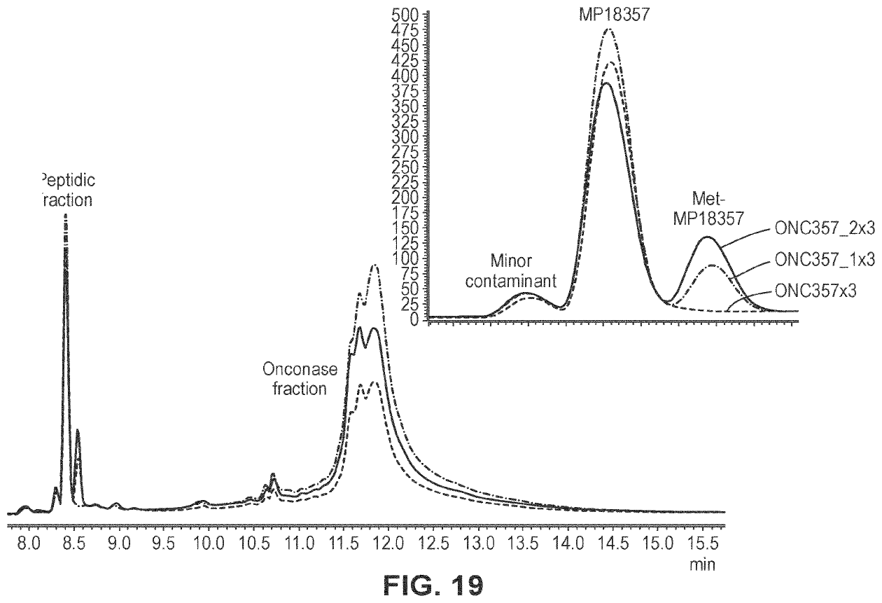

Moreover, HPLC analysis performed after cleavage shows that chemical cleavage was still efficient with additional copies of the oligopeptide at the N and C-termini. (FIG. 19). Indeed, a peak reflecting the single oligopeptide was observed and a smaller peak representing the single oligopeptide with a methionine for constructs 1×3 and 2×3 which comprise N-terminal oligopeptides.

### Example 11: Onconase Variants with Reduced pKa for Increased Peptide Yield

This example describes the evaluation of variant onconase proteins with reduced pKa for increasing peptide yield and purity. Without being bound by theory, decreasing the pI of the onconase protein may reduce the positive charge and thereby reduce the fusion protein tendency to bind to negatively charged nucleic acid. Thus reducing the pI of the onconase protein may increase purity of the fusion protein. Methods

The onconase fusion constructs were expressed in bacteria cells. The bacteria cells were pelleted, after which the cell pellet was lysed through sonication. The cell lysate was pelleted by centrifugation. The pellet was then washed 2 times with TritonX-100 buffer at 37° C. (0.05 M Tris, 2% TritonX-100, pH 7.5), followed by 2× washes with high salt buffer (1 M NaCl, pH 12) and a 5-minute heat shock at 95° C., and finally washed 2× times with water. Inclusion bodies were then recovered by centrifugation after cooling down. The purified inclusion bodies were then solubilized and cleaved in acetic acid. After cleavage, the onconase was precipitated from the solution by addition of NH₄OH buffer (pH 7.0-7.2). The peptide was then lyophilized for ammonium acetate removal and subsequently centrifuged.

### Results

Modification of the onconase sequence to reduce onconase pKa, such as by mutating lysine residues to alanine residues, would likely reduce nucleic acid binding and allow washing at lower pH conditions. FIG. 20A shows an alignment of the onconase protein of SEQ ID NO:1 and a variant with three lysine to alanine substitutions. As shown in FIG. 20B, the variant with three alanine substitutions has a lower pI (8.78 compared to 9.21). Mutating three lysine residues in the onconase to alanine did not affect onconase expression or its ability to function as a fusion partner.

Additional onconase variants are produced that have a lower pI, such as those shown in FIG. 20B. Table 1 shows the estimated pI for the onconase of SEQ ID NO: 1 and the onconase variant comprising three lysine to alanine substitutions. Table 2 shows the predicted pI of the onconase fusion protein constructs. Fusion proteins comprising oligopeptides fused to the onconase variants are expressed and peptides are purified.

TABLE 1
Estimated pI for the onconase of SEQ ID NO: 1 and an onconase
variant comprising three lysine to alanine substitutions.

| Construction | ONCHis_MP18357x1 | ONC_dHK_MP18357x1 |
|---|---|---|
| Estimated pI | 9.21 | 8.78 |

TABLE 2
Estimated pI for onconase fusion proteins.

| Construction | Estimated pI |
|---|---|
| ONCMP1 | 8.78 |
| MP18357x1 | |
| ONCMP2 | 8.31 |
| MP18357x1 | |
| ONCMP3 | 7.56 |
| MP18357x1 | |
| ONCMP4 | 6.95 |
| MP18357x1 | |
| ONCMP5 | 6.16 |
| MP18357x1 | |
| ONCMP6 | 5.50 |
| MP18357x1 | |
| ONCMP7 | 5.10 |
| MP18357x1 | |
| ONCMP8 | 4.87 |
| MP18357x1 | |

Expression levels of fusion proteins and peptides are compared between onconase variants.

### Example 12: Materials for Scalable Biomanufacturing Processes

This example describes raw materials and processes for cost-efficient and highly scalable peptide biomanufacturing processes that have been conducted according to the methods provided herein.

### Results

Peptide production at large industrial scales has been accomplished using the methods provided herein to conduct highly efficient bioprocesses that leverage the physiochemical properties of short, linear, and hydrophilic micropeptides using tailor-made engineered E. coli strains that converted simple nutrients into micropeptides. Well-optimized and highly reliable fermentation processes ensured high peptide expression after 2-stage batch fermentation.

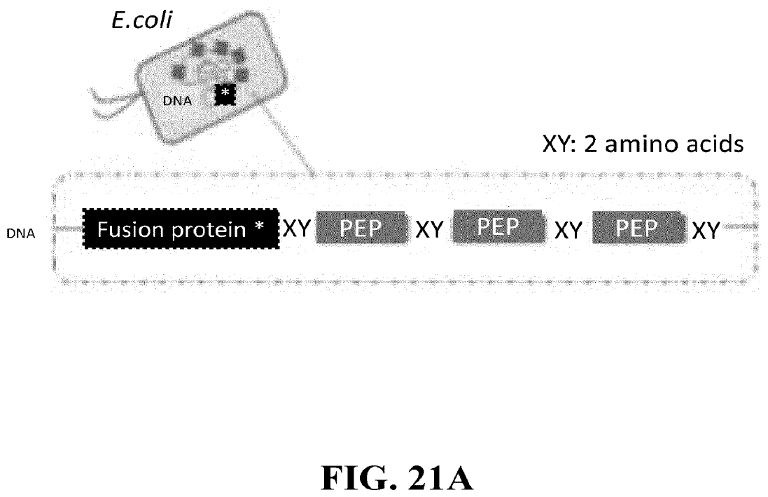

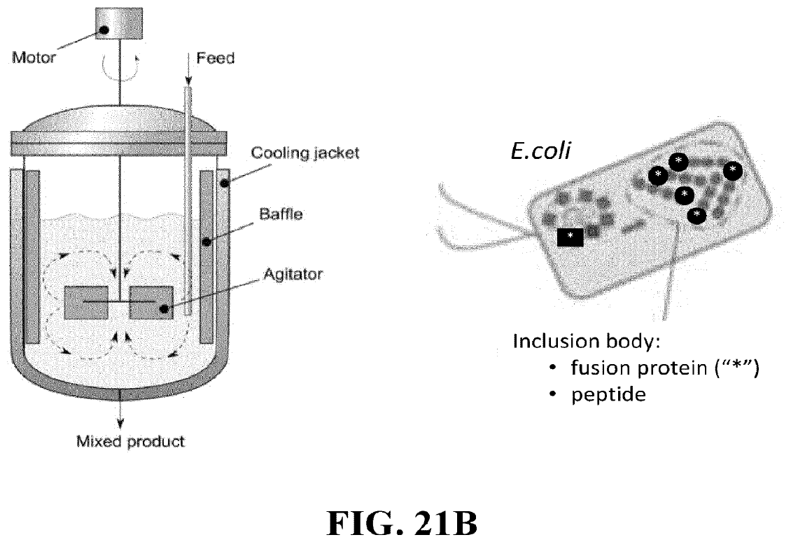

In the first stage, comprising strain engineering (FIG. 21A), inclusion body production lead to highly protected peptides via chromosomal integration in E. coli of multiple copies of a nucleic acid sequence encoding a fusion carrier protein linked by 2-amino acid-long linkers to a concatemer of peptide sequences linked by 2-amino acid-long linkers. The characteristics of the fusion protein were optimized for high peptide expression, yield, and efficient peptide purification. The concatemeric strategy was scaled to maximize peptide expression by including 2-10 copies of the peptide sequences linked by 2-amino acid-long linkers. Inclusion bodies comprised a very stable matrix of the carrier protein and the peptide concatemer. The second stage involved batch growth (FIG. 21B).

SEQUENCE LISTING

| | | SEQ |
|---|---|---|
| Sequence | Sequence | ID |
| description | (N-terminus to C-terminus; or 5 to 3′) | NO: |
| Onconase | MGQEWLTFQKKHITNTREVEYENILSTNLFHYKEKNTFIYSRPEPV | 1 |
| polypeptide | KAILKGIIASKNVLTTSEFYLSEYNVTSRPYKYKLKKSTNKFIVTIEN | |
| | QAPVHFVGGSHHHHHHGTGDP | |
| Onconase N- | MGQEWLTFQKK- | 2 |
| terminus | HITNTREVEYENILSTNLFHYKEKNTFIYSRPEPVKAILKGIIASKNV | |
| sequence | LTTSEFYLSEYNVTSRPYKYKLKKSTNKFIVTIENQAPVHFVGGSH | |
| | HHHHHGTGDP | |
| Onconase | MELHFELLYKW- | 3 |
| variant A N- | HITNTREVEYENILSTNLFHYKEKNTFIYSRPEPVKAILKGIIASKNV | |
| terminus | LTTSEFYLSEYNVTSRPYKYKLKKSTNKFIVTIENQAPVHFVGGSH | |
| sequence | HHHHHGTGDP | |
| Onconase | MASMTGGQQMG- | 4 |
| variant B N- | HITNTREVEYENILSTNLFHYKEKNTFIYSRPEPVKAILKGIIASKNV | |
| terminus | LTTSEFYLSEYNVTSRPYKYKLKKSTNKFIVTIENQAPVHFVGGSH | |
| sequence | HHHHHGTGDPW | |
| Onconase | MRRKKRPDPDSW- | 5 |
| variant C N- | HITNTREVEYENILSTNLFHYKEKNTFIYSRPEPVKAILKGIIASKNV | |
| terminus | LTTSEFYLSEYNVTSRPYKYKLKKSTNKFIVTIENQAPVHFVGGSH | |
| sequence | HHHHHGTGDP | |
| Onconase | MTKEAERLDVP- | 6 |
| variant D N- | HITNTREVEYENILSTNLFHYKEKNTFIYSRPEPVKAILKGIIASKNV | |
| terminus | LTTSEFYLSEYNVTSRPYKYKLKKSTNKFIVTIENQAPVHFVGGSH | |
| sequence | HHHHHGTGDPW | |
| Onconase | MPEEVFHGDAHW- | 7 |
| variant E N- | HITNTREVEYENILSTNLFHYKEKNTFIYSRPEPVKAILKGIIASKNV | |
| terminus | LTTSEFYLSEYNVTSRPYKYKLKKSTNKFIVTIENQAPVHFVGGSH | |
| sequence | HHHHHGTGDP | |
| Onconase | MTHTIKAMEPKW- | 8 |
| variant F N- | HITNTREVEYENILSTNLFHYKEKNTFIYSRPEPVKAILKGIIASKNV | |
| terminus | LTTSEFYLSEYNVTSRPYKYKLKKSTNKFIVTIENQAPVHFVGGSH | |
| sequence | HHHHHGTGDP | |
| Onconase | MERHVVEQQLW- | 9 |
| variant G- | HITNTREVEYENILSTNLFHYKEKNTFIYSRPEPVKAILKGIIASKNV | |
| terminus | LTTSEFYLSEYNVTSRPYKYKLKKSTNKFIVTIENQAPVHFVGGSH | |
| sequence | HHHHHGTGDP | |
| Onconase | MKRRVILFSSQW- | 10 |
| variant H N- | HITNTREVEYENILSTNLFHYKEKNTFIYSRPEPVKAILKGIIASKNV | |
| terminus | LTTSEFYLSEYNVTSRPYKYKLKKSTNKFIVTIENQAPVHFVGGSH | |
| sequence | HHHHHGTGDP | |
| Chemical | DP | 11 |
| cleavage linker | | |
| sequence | | |
| MP18357 | KKEEMSYMKR | 12 |
| MP18366 | MEGNGTLSFA | 13 |
| MP18913 | PKKEEMSYMKRD | 14 |
| Concatemeric | MELNHFELLYKWHITNTREVEYENILSTNLFHYK | 15 |
| onconase fusion | EKNTFIYSRPEPVKAILKGIIASKNVLTTSEFYLSEY | |
| construct A | NVTSRPYKYKLKKSTNKFIVTIENQAPVH | |
| | FVGGSHHHHHHGTGDPKKEEMSYMKR* | |
| Concatemeric | MASMTGGQQMGWHITNTREVEYENILSTNLFH | 16 |
| onconase fusion | YKEKNTFIYSRPEPVKAILKGIIASKNVLTTSEFY | |
| construct B | LSEYNVTSRPYKYKLKKSTNKFIVTIENQAPVH | |
| | FVGGSHHHHHHGTGDPKKEEMSYMKR* | |
| Concatemeric | MRRKKRPDPDSWHITNTREVEYENILSTNLFHYK | 17 |
| onconase fusion | EKNTFIYSRPEPVKAILKGIIASKNVLTTSEFYLSEY | |
| construct C | NVTSRPYKYKLKKSTNKFIVTIENQAPVHFVGGSH | |
| | HHHHHGTGDPKKEEMSYMKR* | |
| Concatemeric | MTKEAERLDVPWHITNTREVEYENILSTNLFHYK | 18 |
| onconase fusion | EKNTFIYSRPEPVKAILKGIIASKNVLTTSEFYLSEY | |
| construct D | NVTSRPYKYKLKKSTNKFIVTIENQAPVHFVGGSH | |
| | HHHHHGTGDPKKEEMSYMKR* | |
| Concatemeric | MPEEVFHGDAHWHITNTREVEYENILSTNLFHYK | 19 |
| onconase fusion | EKNTFIYSRPEPVKAILKGIIASKNVLTTSEFYLSEY | |
| construct E | NVTSRPYKYKLKKSTNKFIVTIENQAPVHFVGGSHH | |
| | HHHHGTGDPKKEEMSYMKR* | |
| Concatemeric | MTHTIKAMEPFWHITNTREVEYENILSTNLFHYK | 20 |
| onconase fusion | EKNTFIYSRPEPVKAILKGIIASKNVLTTSEFYLSEY | |
| construct F | NVTSRPYKYKLKKSTNKFIVTIENQAPVHFVGGSH | |
| | HHHHHGTGDPKKEEMSYMKR* | |
| Concatemeric | MERHVVEQQLWHITNTREVEYENILSTNLFHYKEK | 21 |
| onconase fusion | NTFIYSRPEPVKAILKGIIASKNVLTTSEFYLSEYNV | |
| construct G | TSRPYKYKLKKSTNKFIVTIENQAPVHFVGGSHHH | |
| | HHHGTGDPKKEEMSYMKR* | |
| Concatemeric | MKRRVILFSSQWHITNTREVEYENILSTNLFHYKEK | 22 |
| onconase fusion | NTFIYSRPEPVKAILKGIIASKNVLTTSEFYLSEYNV | |
| construct H | TSRPYKYKLKKSTNKFIVTIENQAPVHFVGCGSHH | |
| | HHHHGTGDPKKEEMSYMKR* | |
| TAF12 | MVLTKKKLQELVREVAPNEQLEEEVEEMLLQIAEEFIESVVTAAAQ | 23 |
| | LARHRKSSTLEVKEVQLHLERQWNMWL | |
| PurF | MGIVGIAGVMPVNQSIYDALTVLQHRGQDAAGIITIDANNCFRLRK | 24 |
| | ANGLVSDVFEARHMQRLQGNMGIGHVRYPTAGSSSASEAQPFYV | |
| | NSPYGITLAHNGNLTNAHELRKKLFEEKRRHINTTSDSEILLNIFAS | |
| | ELDNFRHYPLEA | |
| OmpX | MATSTVTGGYAQSDAQGQMNKMGGFNLKYRYEEDNSPLGVIGSF | 25 |
| | TYTEKSRTASSGDYNKNQYYGITAGPAYRINDWASIYGVVGVGYG | |
| | KFQTTEYPTYKHDTSDYGFSYGAGLQFNPMENVALDFSYEQSRIRS | |
| | VDVGTWIAGVGYRL | |
| KSI | MHTPEHITAVVQRFVAALNAGDLDGIVALFADDATVEEPVGSEPR | 26 |
| | SGTAAIREFYANSLKLPLAVELTQEVRAVANEAAFAFTVSFEYQGR | |
| | KTVVAPIDHFRFNGAGKVVSIRALFGEKNIHACQML | |
| Linker Sequence | GGSGTG | 27 |
| Linker 2 | GGGS | 28 |
| Linker 3 | GGG | 29 |
| Linker 4 | GSGS | 30 |
| Linker 5 | GSGSGGT | 31 |
| Linker 6 | GTGSGTG | 32 |
| Linker 7 | EAAAK | 33 |
| Additional | See supplemental sequence listing, which includes under <223> for each | 34- |
| peptides | sequence: family function, peptide family, and a description | 2401 |

1.-179. (canceled)

180. A method of producing oligopeptides in a bacteria cell, comprising:
expressing a fusion polypeptide comprising an insoluble carrier polypeptide operably linked to two or more oligopeptides by a peptide bond, and
releasing the two or more oligopeptides from the insoluble carrier polypeptide by sequence-specific chemical cleavage of the peptide bond,
wherein the insoluble carrier polypeptide comprises a TAF12 polypeptide that consists of SEQ ID NO:23, or a modified TAF12 polypeptide defined as having an amino acid sequence having at least 90%, at least 91%, at least 92%, at least 93%, at least 94%, at least 95%, at least 96%, at least 97%, at least 98%, at least 99% to SEQ ID NO:23.

181. The method of claim 180, wherein the different sequence-specific chemical cleavage is an acidic cleavage.

182. The method of claim 181, wherein the acid is a strong acid.

183. The method of claim 182, wherein the acid is sulfuric acid.

184. The method of claim 180, wherein the peptide bond is an Asp-Pro bond, whereby the released oligopeptides optionally comprise an N-terminal proline or a C-terminal aspartic acid or comprise both an N-terminal proline and the C-terminal aspartic acid.

185. The method of claim 180, wherein the fusion polypeptide comprises three or more oligopeptides, four or more oligopeptides, five or more oligopeptides, six or more oligopeptides, eight or more oligopeptides, ten or more oligopeptides, fifteen or more oligopeptides, or twenty or more oligopeptides.

186. The method of claim 180, wherein all the oligopeptides are operably linked to the C-terminus of the insoluble carrier polypeptide.

187. The method of claim 180 wherein the bacteria is E. coli.

188. The method of claim 187, wherein the bacteria is a BL21 E. coli.

189. The method of claim 180, comprising a step of purifying the inclusion bodies formed by the fusion polypeptide in the bacteria cell, wherein the step of purifying the inclusion bodies comprises
lysing the cell to form a cell lysate,
centrifuging the cell lysate to form a pellet,
washing the pellet in a surfactant buffer comprising a nonionic surfactant at least once, at least twice, or at least three times,
washing the pellet in a salt buffer comprising at least 0.5M NaCl at least once, at least twice, or at least three times, and
washing the pellet in water at least once, at least twice, or at least three times, and thereby producing purified inclusion bodies.

190. The method of claim 189, comprising a step of purifying the inclusion bodies formed by the fusion polypeptide in the bacteria cell and a subsequent step of incubating the inclusion bodies with acid at a temperature of greater than 50° C., for at least one hour, wherein the oligopeptide is released from the fusion polypeptide by sequence-specific cleavage of the peptide bond.

191. The method of claim 190, wherein the step of incubating the inclusion bodies with acid is conducted at a temperature of greater than 80° C.

192. The method of claim 190, wherein the step of incubating the inclusion bodies with acid is conducted at a temperature at a pH below 3.0, preferably from 2.6 to 2.8.

193. The method of claim 180, wherein the cleavage takes place for about 1-24 hours at a pH of about 2-3.5 and a temperature of about 70° C.-90° C.

194. A fusion polypeptide comprising an insoluble carrier polypeptide operably linked to two or more oligopeptides, wherein the operably linkage between the two or more oligopeptides and the insoluble carrier polypeptide comprise a peptide bond that is capable of sequence-specific chemical cleavage, wherein the insoluble carrier polypeptide comprises a TAF12 polypeptide that consists of SEQ ID NO:23, or a modified TAF12 polypeptide defined as having an amino acid sequence having at least 90%, at least 91%, at least 92%, at least 93%, at least 94%, at least 95%, at least 96%, at least 97%, at least 98%, at least 99% to SEQ ID NO:23.

195. The fusion polypeptide of claim 194, wherein the different sequence-specific chemical cleavage is an acidic cleavage, and wherein the peptide bond is preferably an Asp-Pro bond.

196. The fusion polypeptide of claim 194, wherein the fusion polypeptide comprises three or more oligopeptides, four or more oligopeptides, five or more oligopeptides, six or more oligopeptides, eight or more oligopeptides, ten or more oligopeptides, fifteen or more oligopeptides, or twenty or more oligopeptides.

197. The fusion polypeptide of claim 194, wherein all the oligopeptides are operably linked to the C-terminus of the insoluble carrier polypeptide.

198. A nucleic acid encoding the fusion polypeptide as defined in claim 194.

199. A bacterial cell comprising a nucleic acid encoding the fusion polypeptide as defined in claim 194.

200. The bacterial cell of claim 199, wherein the bacterial cell is a E. coli cell.

201. The bacterial cell of claim 200, wherein the bacterial cell is a BL21 E. coli cell.
